(19) United States
(12) Patent Application Publication
(10) Pub. No.: US 2010/0215618 A1
(43) Pub. Date: Aug. 26, 2010

(54) NOVEL COMPOUNDS - 644

(75) Inventors: Malcolm Clive Carter, London (GB); Neil Mathews, London (GB)

Correspondence Address: ASTRAZENECA R&D BOSTON, 35 GATEHOUSE DRIVE, WALTHAM, MA, 02451-1215, US

(73) Assignee: Arrow Therapeutics Limited, London (GB)

(21) Appl. No.: 12/710,647

(22) Filed: Feb. 23, 2010

Related U.S. Application Data
(60) Provisional application No. 61/154,460, filed on Feb. 23, 2009.
(60) Provisional application No. 61/250,073, filed on Oct. 9, 2009.

(51) Int. Cl. A61K 38/21; C07D 279/12; A61K 31/541; C07D 403/12; A61K 31/496; A61P 1/16; C07D 417/02; C07D 413/02

(52) U.S. Cl. 424/85.4; 544/58.2; 514/227.8; 544/370; 514/254.05; 514/235.5; 544/60; 544/121

(57) ABSTRACT

The invention provides compounds of formula (I)

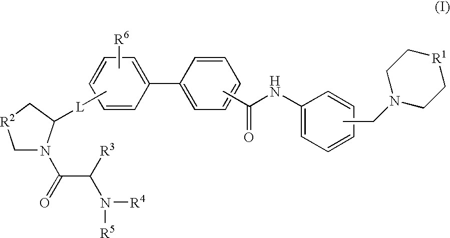

wherein R¹, R², R³, R⁴, R⁵, R⁶ and L are as defined in the specification and optical isomers, racemates and tautomers thereof, and pharmaceutically acceptable salts thereof; together with processes for their preparation, pharmaceutical compositions containing them and their use in therapy. The compounds are useful in the treatment of hepatitis C virus.

This application claims the benefit under 35 U.S.C. §119(e) of Application No. 61/154,460 filed 23 Feb. 2009 and Application No. 61/250,073 filed 9 Oct. 2009.

The present invention relates to novel compounds, processes for their preparation, pharmaceutical compositions containing them and their use in therapy. In particular, the compounds are useful for the treatment or prevention of Flaviviridae infections, particularly hepatitis C virus (HCV), in a warm-blooded animal, such as man.

Hepatitis C virus (HCV) is a positive single-stranded RNA virus classified within the Flaviviridae family and identified as the etiological agent responsible for non-A and non-B hepatitis in 1989 (Choo Q-L et al. Science 1989; 244:359-62). Based on nucleotide sequence up to eleven different major genotypes of HCV have been defined (Simmonds P et al. Hepatology 2005; 42:962-73). HCV genotypes can be sub-divided further with genotypes 1a, 1b and 2a most prevalent in North America, Europe, Japan and China.

It is estimated that approximately 170 million people are infected worldwide; 3% of the world's population. While the disease does spontaneously resolve in approximately 20% of patients, for the majority the infection becomes chronic. Chronic HCV infection is a significant public health problem. Viral replication is associated with necro-inflammatory activity in the liver that eventually results in the development of cirrhosis and hepatocellular carcinoma in significant numbers of patients and is recognised as the leading indication for liver transplant in the developed world (Seeff L B. Hepatology 2002; 36(5 Suppl 1):S35-46.). In the majority of patients the acute phase of infection is asymptomatic until liver function abnormalities are noted during routine healthcare checks or when severe liver damage has occurred. Lack of symptoms and minor liver enzyme elevations are typical of HCV infection and cannot be taken as evidence of lack of disease progression. Major risk factors associated with progressive liver disease include male gender, ethnicity, alcohol abuse, HIV/HCV co-infection, age greater than 40 years at infection and pre-existing fibrosis.

The current standard of care (SoC) for the treatment of HCV infection is pegylated interferon in combination with a broad spectrum antiviral agent, ribavirin (Chandler G. et al. Hepatology 2002; 36:S135-S144.). Cure is achievable and the indicator of this is a sustained virological response (SVR), defined as HCV RNA negativity 24 weeks after the end of treatment. Patients who achieve SVR have been shown to have a low likelihood of relapse and a favourable long term prognosis. Efficacy rates, measured as SVR, are highest in patients with genotypes 2 and 3 at approximately 88%. Less than 50% of patients with genotypes 1, 4, 5 and 6 achieve SVR after 48 weeks of therapy.

The current SoC is contra-indicated in significant numbers of HCV patients, e.g. those with advanced liver disease or pre-existing psychiatric illness. It is poorly tolerated and frequently leads to the need for dose reductions, poor compliance, or the need for premature discontinuation of therapy, all of which reduce cure rates. All patients suffer from adverse effects, most frequently flu-like symptoms, myalgia, fatigue, gastrointestinal disturbances, psychiatric disorders and haematological abnormalities. Adverse effects are managed either with supportive measures and/or adjustment of SoC dosage. However, 10-14% of patients discontinue treatment and significant numbers of diagnosed patients are currently ‘warehoused’ waiting for more tolerable therapies of shorter duration and higher efficacy.

In patients with chronic HCV infection, clearance of virus has been shown to significantly reduce the disease progression. Hence there is significant unmet need for better tolerated and higher efficacy regimes to treat patients with chronic HCV.

HCV replicates very poorly in tissue culture and several surrogate models are currently used to determine anti-HCV activity in vitro Inhibitors of viral RNA replication can be screened on hepatocellular carcinoma cell lines harbouring an HCV replicon. Cells are stably transfected with self-replicating subgenomic viral RNAs and a reporter gene readout can be used to evaluate the efficacy of potential anti-HCV compounds. Activity against the replicon system is a good predictor of HCV load reductions in clinical evaluations in man (Hinrichsen H, et al. Gastroenterology 2004; 127(5):1347-55.; Reesink H W, et al. Gastroenterology 2006; 131:997-1002.).

The present invention provides a series of novel compounds which have activity in the HCV replicon system against genotypes 1a and 1b, and are therefore expected to inhibit viral replication in man.

In one aspect, the present invention provides a compound of formula (I), or a pharmaceutically acceptable salt thereof,

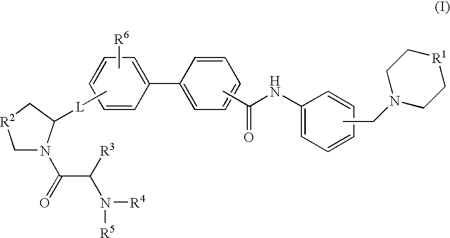

wherein
L represents a five membered heteroaromatic ring containing 1 to 3 heteroatoms independently selected from O, S and N;
R¹ represents SO₂, NSO₂R⁷ or NSO₂NR⁷R⁸;
R² represents a bond, CH₂, CH₂CH₂ or CH₂O;
R³ represents H, C1-4 alkyl, CH₂OH, CHOHCH₃ or Ph;
R⁴ represents H, C1-4 alkyl or CO₂R⁹;
R⁵ represents H or C1-4 alkyl;
R⁶ represents H, C1-2 alkyl, halogen or OCF₃;
R⁷ represents C1-4 alkyl; and
R⁸ and R⁹ independently represent H or C1-4 alkyl.

In another aspect, the present invention provides a compound of formula (I), or a is pharmaceutically acceptable salt thereof,

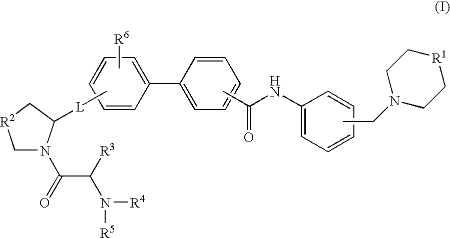

wherein
L represents a five membered heteroaromatic ring containing 1 to 3 heteroatoms independently selected from O, S and N;
R¹ represents SO₂, NSO₂R⁷ or NSO₂NR⁷R⁸;
R² represents a bond, CH₂, CH₂CH₂ or CH₂O;
R³ represents C1-4 alkyl, CH₂OH or Ph;
R⁴ represents H, C1-4 alkyl or CO₂R⁹;
R⁵ represents H or C1-4 alkyl;
R⁶ represents H, C1-2 alkyl, halogen or OCF₃;
R⁷ represents C1-4 alkyl; and
R⁸ and R⁹ independently represent H or C1-4 alkyl.

In the context of the present application, an alkyl moiety may be linear or branched.

L represents a five membered heteroaromatic ring containing 1 to 3 heteroatoms independently selected from O, S and N. Examples of such a ring include imidazole, oxazole, thiazole, pyrazole, triazole and oxadiazole.

In one embodiment, L represents an imidazole ring. In another embodiment, L represents a 2,4-disubstituted imidazole ring.

In another embodiment, R¹ represents NSO₂R⁷. In another embodiment, R¹ represents NSO₂R⁷ and R⁷ represents 1-propyl.

When R² represents a bond, the ring containing R² represents an azetidine ring.

When R² represents CH₂, the ring containing R² represents a pyrrolidine ring.

When R² represents CH₂CH₂, the ring containing R² represents a piperidine ring.

When R² represents CH₂O, the ring containing R² represents a morpholine ring.

In one embodiment, R² represents CH₂ and the ring containing R² represents a pyrrolidine ring.

R³ represents H, C1-4 alkyl (e.g. methyl, ethyl, 1-propyl, 2-propyl, n-butyl, iso-butyl sec-butyl or tert-butyl), CH₂OH, CHOHCH₃ or Ph. In one embodiment, R³ represents C1-4 alkyl (e.g. methyl, ethyl, 1-propyl, 2-propyl, n-butyl, iso-butyl or tert-butyl), CH₂OH or Ph. In another embodiment, R³ represents 2-propyl. In yet another embodiment, R³ represents phenyl.

In one embodiment, R⁴ represents H, C1-4 alkyl (e.g. methyl, ethyl, 1-propyl, 2-propyl, n-butyl, iso-butyl or tert-butyl) or CO₂—C1-4 alkyl (e.g. CO₂-methyl, CO₂-ethyl, CO₂-1-propyl, CO₂-2-propyl, CO₂-n-butyl, CO₂-iso-butyl or CO₂-tert-butyl). In one embodiment, R⁴ represents CO₂R⁹, where R⁹ represents H or C1-4 alkyl. In one embodiment, R⁴ represents CO₂—C1-4 alkyl. In one embodiment, R⁴ represents CO₂-methyl. In one embodiment, R⁴ represents CO₂-tert-butyl.

R⁵ represents H or C1-4 alkyl (e.g. methyl, ethyl, 1-propyl, 2-propyl, n-butyl, iso-butyl or tert-butyl). In one embodiment, R⁵ represents H.

In one embodiment, R⁴ represents CO₂-methyl or CO₂-tert-butyl and R⁵ represents H.

R⁶ represents H, C1-2 alkyl (e.g. methyl or ethyl), halogen (e.g. fluoro, chloro, bromo or iodo) or OCF₃. In one embodiment, R⁶ represents H. In another embodiment, R⁶ represents a methyl substituent at the ortho position of the phenyl ring relative to the bond to the second phenyl ring of the bi-phenyl core.

In one embodiment, L represents an imidazole ring; R¹ represents SO₂, NSO₂R⁷ or NSO₂NR⁷R⁸; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁸ and R⁹ independently represent H or C1-4 alkyl.

In one embodiment, L represents an imidazole ring; R¹ represents SO₂; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; and R⁹ represents H or C1-4 alkyl.

In one embodiment, L represents an imidazole ring; R¹ represents NSO₂R⁷, particularly NSO₂— propyl; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-C4 alkyl; R⁶ represents H, C1-2 alkyl, halogen or OCF₃; R⁷ represents C1-4 alkyl; and R⁹ represents H or C1-4 alkyl.

In one embodiment, L represents an imidazole ring; R¹ represents SO₂ or NSO₂R⁷; R² represents CH₂; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁹ represents H or C1-4 alkyl.

In one embodiment, L represents an imidazole ring; R¹ represents SO₂ or NSO₂R⁷; R² represents CH₂; R³ represents C1-4 alkyl, CH₂OH or Ph; R⁴ represents CO₂R⁹; R⁵ represents H or C1-4 alkyl; R⁶ represents H, C1-2 alkyl, halogen or OCF₃; R⁷ represents C1-4 alkyl; and R⁹ represents H or C1-4 alkyl.

In one embodiment, L represents an imidazole ring; R¹ represents SO₂ or NSO₂R⁷; R² represents CH₂; R³ represents C1-4 alkyl, CH₂OH or Ph; R⁴ represents CO₂R⁹; R⁵ represents H; R⁶ represents H; R⁷ represents C1-4 alkyl; and R⁹ represents C1-4 alkyl.

In one embodiment, L represents an imidazole ring; R¹ represents SO₂; R² represents CH₂; R³ represents C1-4 alkyl or Ph; R⁴ represents CO₂R⁹; R⁵ represents H; R⁶ represents H; and R⁹ represents C1-4 alkyl.

In another aspect, the present invention provides a compound of formula (Ia), or a pharmaceutically acceptable salt thereof,

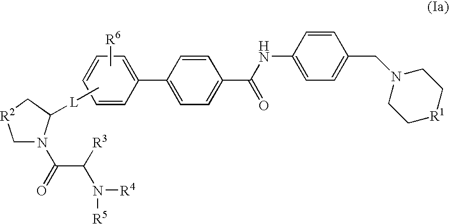

wherein L; R¹; R²; R³; R⁴; R⁵; R⁶; R⁷; R⁸; and R⁹ are as defined for formula (I).

In one embodiment the invention relates to compounds of formula (Ia) wherein L represents an imidazole ring; R¹ represents SO₂, NSO₂R⁷ or NSO₂NR⁷R⁸; R² represents is a bond, CH₂, CH₂CH₂ or CH₂O; R³ represents H, C1-4 alkyl, CH₂OH, CHOHCH₃, or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁸ and R⁹ independently represent H or C1-4 alkyl.

In one embodiment the invention relates to compounds of formula (Ia) wherein L represents an imidazole ring; R¹ represents SO₂, NSO₂R⁷ or NSO₂NR⁷R⁸; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁸ and R⁹ independently represent H or C1-4 alkyl.

In one embodiment the invention relates to compounds of formula (Ia) wherein L represents an imidazole ring; R¹ represents SO₂; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; and R⁹ represents H or C1-4 alkyl.

In one embodiment the invention relates to compounds of formula (Ia) wherein L represents an imidazole ring; R¹ represents NSO₂R⁷, particularly NSO₂-propyl; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁹ represents H or C1-4 alkyl.

In one embodiment the invention relates to compounds of formula (Ia) wherein L is represents an imidazole ring; R¹ represents SO₂ or NSO₂R⁷; R² represents CH₂; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁹ represents H or C1-4 alkyl.

In one embodiment the invention relates to compounds of formula (Ia) wherein L represents an imidazole ring; R¹ represents SO₂ or NSO₂R⁷; R² represents CH₂; R³ represents C1-4 alkyl, CH₂OH or Ph; R⁴ represents CO₂R⁹; R⁵ represents H or C1-4 alkyl; R⁶ represents H, C1-2 alkyl, halogen or OCF₃; R⁷ represents C1-4 alkyl; and R⁹ represents H or C1-4 alkyl.

In one embodiment the invention relates to compounds of formula (Ia) wherein L represents an imidazole ring; R¹ represents SO₂ or NSO₂R⁷; R² represents CH₂; R³ represents C1-4 alkyl, CH₂OH or Ph; R⁴ represents CO₂R⁹; R⁵ represents H; R⁶ represents H; R⁷ represents C1-4 alkyl; and R⁹ represents C1-4 alkyl.

In one embodiment the invention relates to compounds of formula (Ia) wherein L represents an imidazole ring; R¹ represents SO₂; R² represents CH₂; R³ represents C1-4 alkyl or Ph (particularly C1-4 alkyl); R⁴ represents CO₂R⁹; R⁵ represents H; R⁶ represents H; and R⁹ represents C1-4 alkyl.

In another aspect, the present invention provides a compound of formula (Ib), or a pharmaceutically acceptable salt thereof,

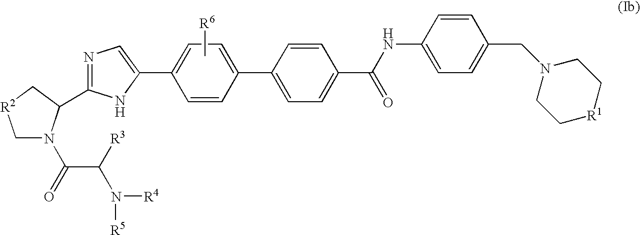

wherein R¹; R²; R³; R⁴; R⁵; R⁶; R⁷; R⁸; and R⁹ are as defined for formula (I).

In one embodiment the invention relates to compounds of formula (Ib) wherein R¹ represents SO₂, NSO₂R⁷ or NSO₂NR⁷R⁸; R² represents a bond, CH₂, CH₂CH₂ or CH₂O; R³ represents H, C1-4 alkyl, CH₂OH, CHOHCH₃, or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁸ and R⁹ independently represent H or C1-4 alkyl.

In one embodiment the invention relates to compounds of formula (Ib) wherein R¹ represents SO₂, NSO₂R⁷ or NSO₂NR⁷R⁸; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁸ and R⁹ independently represent H or C1-4 alkyl.

In one embodiment the invention relates to compounds of formula (Ib) wherein R¹ represents SO₂; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; and R⁹ represents H or C1-4 alkyl.

In one embodiment the invention relates to compounds of formula (Ib) wherein R¹ represents NSO₂R⁷, particularly NSO₂— propyl; R² represents a bond, CH₂, CH₂CH₂ or CH₂O; R³ represents C1-4 alkyl, CH₂OH or Ph; R⁴ represents H, C1-4 alkyl or CO₂R⁹; R⁵ represents H or C1-4 alkyl; R⁶ represents H, C1-2 alkyl, halogen or OCF₃; R⁷ represents C1-4 alkyl; and R⁹ represents H or C1-4 alkyl.

In one embodiment the invention relates to compounds of formula (Ib) wherein R¹ represents SO₂ or NSO₂R⁷; R² represents CH₂; R³ represents C1-4 alkyl, CH₂OH or Ph; R⁴ represents H, C1-4 alkyl or CO₂R⁹; R⁵ represents H or C1-4 alkyl; R⁶ represents H, is C1-2 alkyl, halogen or OCF₃; R⁷ represents C1-4 alkyl; and R⁹ represents H or C1-4 alkyl.

In one embodiment the invention relates to compounds of formula (Ib) wherein R¹ represents SO₂ or NSO₂R⁷; R² represents CH₂; R³ represents C1-4 alkyl, CH₂OH or Ph; R⁴ represents CO₂R⁹; R⁵ represents H or C1-4 alkyl; R⁶ represents H, C1-2 alkyl, halogen or OCF₃; R⁷ represents C1-4 alkyl; and R⁹ represents H or C1-4 alkyl.

In one embodiment the invention relates to compounds of formula (Ib) wherein R¹ represents SO₂ or NSO₂R⁷; R² represents CH₂; R³ represents C1-4 alkyl, CH₂OH or Ph; R⁴ represents CO₂R⁹; R⁵ represents H; R⁶ represents H; R⁷ represents C1-4 alkyl; and R⁹ represents C1-4 alkyl.

In one embodiment the invention relates to compounds of formula (Ib) wherein R¹ represents SO₂; R² represents CH₂; R³ represents C1-4 alkyl or Ph (particularly C1-4 alkyl); R⁴ represents CO₂R⁹; R⁵ represents H; R⁶ represents H; and R⁹ represents C1-4 alkyl.

Examples of compounds of the invention include:
tert-butyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
tert-butyl N-[(1R)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxoethyl]carbamate;
Methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2,2-dimethyl-propyl]carbamate;
Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
Methyl N-[(1R)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
4-[4-[2-[(2S)-1-[(2R)-2-(diethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
4-[4-[2-[(2S)-1-[(2R)-2-(dimethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
4-[4-[2-[(2S)-1-[(2S)-2-amino-3-methyl-butanoyl]pyrrolidin-2-yl]-1H-imidazol-5-yl]phenyl]-N-[4-[(1,1-dioxo-1,4-thiazinan-4-yl)methyl]phenyl]benzamide;
Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-(hydroxymethyl)-2-oxo-ethyl]carbamate;
tert-Butyl N-[2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-ethyl]carbamate;
Methyl N-[(1R)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-3-methyl-butyl]carbamate;
Methyl N-[(1R)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-3-methyl-butyl]carbamate;
Methyl N-[(1S,2S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-butyl]carbamate;
Methyl N-[(1S,2R)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-hydroxy-propyl]carbamate;
Methyl N-[(1R,2S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-hydroxy-propyl]carbamate;
Methyl N-[(1R)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-(hydroxymethyl)-2-oxo-ethyl]carbamate;
Methyl N-[(1S)-1-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-2-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxo-ethyl]carbamate;
Methyl N-[(1S)-1-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2,2-dimethyl-propyl]carbamate;
Methyl N-[(1S)-2-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
Methyl N-[(1R)-2-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
4-[4-[2-[(2R)-1-[(2R)-2-(diethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
4-[4-[2-[(2R)-1-[(2R)-2-(dimethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
methyl N-[(1R)-1-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-1-[(2S)-2-[5-[4-[3-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-2-[(2S)-2-[5-[4-[3-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxo-ethyl]carbamate;
Methyl N-[(1S)-2-[(2S)-2-[5-[4-[3-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
Methyl N-[(1R)-2-[(2S)-2-[5-[4-[3-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
3-[4-[2-[(2S)-1-[(2R)-2-(diethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
Methyl N-[(1S)-1-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-2-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxo-ethyl]carbamate;
Methyl N-[(1S)-1-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2,2-dimethyl-propyl]carbamate;
Methyl N-[(1S)-2-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
Methyl N-[(1R)-2-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
4-[3-[2-[(2S)-1-[(2R)-2-(diethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
4-[3-[2-[(2S)-1-[(2R)-2-(dimethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
Methyl N-[(1S)-2-methyl-1-[(2S)-2-[5-[4-[4-[[4-[(4-propylsulfonylpiperazin-1-yl)methyl]phenyl]carbamoyl]phenyl]phenyl]-1H-imidazol-2-yl]pyrrolidine-1-carbonyl]propyl]carbamate;
Methyl N-[(1S)-1-methyl-2-oxo-2-[(2S)-2-[5-[4-[4-[[4-[(4-propylsulfonylpiperazin-1-yl)methyl]phenyl]carbamoyl]phenyl]phenyl]-1H-imidazol-2-yl]pyrrolidin-1-yl]ethyl]carbamate;
Methyl N-[(1S)-2-oxo-1-phenyl-2-[(2S)-2-[5-[4-[4-[[4-[(4-propylsulfonylpiperazin-1-yl)methyl]phenyl]carbamoyl]phenyl]phenyl]-1H-imidazol-2-yl]pyrrolidin-1-yl]ethyl]carbamate;
Methyl N-[(1R)-2-oxo-1-phenyl-2-[(2S)-2-[5-[4-[4-[[4-[(4-propylsulfonylpiperazin-1-yl)methyl]phenyl]carbamoyl]phenyl]phenyl]-1H-imidazol-2-yl]pyrrolidin-1-yl]ethyl]carbamate;
4-[4-[2-[(2S)-1-[(2R)-2-(diethylamino)-2-phenyl-acetyl]pyrrolidin-2-yl]-1H-imidazol-5-yl]phenyl]-N-[4-[(4-propylsulfonylpiperazin-1-yl)methyl]phenyl]benzamide;
4-[4-[2-[(2S)-1-[(2R)-2-(dimethylamino)-2-phenyl-acetyl]pyrrolidin-2-yl]-1H-imidazol-5-yl]phenyl]-N-[4-[(4-propylsulfonylpiperazin-1-yl)methyl]phenyl]benzamide;
Methyl N-[(1S)-1-[(3R)-3-[5-[4-[4-[[4-[(1,1-dioxo-1,4-thiazinan-4-yl)methyl]phenyl]carbamoyl]phenyl]phenyl]-1H-imidazol-2-yl]morpholine-4-carbonyl]-2-methyl-propyl]carbamate;
Methyl N-[(1S)-2-[(3R)-3-[5-[4-[4-[[4-[(1,1-dioxo-1,4-thiazinan-4-yl)methyl]phenyl]carbamoyl]phenyl]phenyl]-1H-imidazol-2-yl]morpholin-4-yl]-2-oxo-1-phenyl-ethyl]carbamate;
tert-Butyl N-[(1S)-2-[(3R)-3-[5-[4-[4-[[4-[(1,1-dioxo-1,4-thiazinan-4-yl)methyl]phenyl]carbamoyl]phenyl]phenyl]-1H-imidazol-2-yl]morpholin-4-yl]-2-oxo-1-phenyl-ethyl]carbamate;
4-[4-[2-[(3R)-4-[(2R)-2-(dimethylamino)-2-phenyl-acetyl]morpholin-3-yl]-1H-imidazol-5-yl]phenyl]-N-[4-[(1,1-dioxo-1,4-thiazinan-4-yl)methyl]phenyl]benzamide;
methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]piperidine-1-carbonyl]-2-methyl-propyl]carbamate;
Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]-1-piperidyl]-2-oxo-1-phenyl-ethyl]carbamate;
tert-Butyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]-1-piperidyl]-2-oxo-1-phenyl-ethyl]carbamate;
4-[4-[2-[(2S)-1-[(2R)-2-(dimethylamino)-2-phenyl-acetyl]-2-piperidyl]-1H-imidazol-5-yl]phenyl]-N-[4-[(1,1-dioxo-1,4-thiazinan-4-yl)methyl]phenyl]benzamide;
Methyl N-[(1S)-1-[(2S)-2-[5-[4-[4-[[4-[(1,1-dioxo-1,4-thiazinan-4-yl)methyl]phenyl]carbamoyl]phenyl]-3-methyl-phenyl]-1H-imidazol-2-yl]pyrrolidine-1-carbonyl]-2-methyl-propyl]carbamate;
   and pharmaceutically acceptable salts thereof.

Compounds of formulae (I), (Ia) and (Ib) are capable of existing in stereoisomeric forms. It will be understood that the invention encompasses the use of all geometric and optical isomers of the compounds of formulae (I), (Ia) and (Ib) and mixtures thereof, including racemates. The use of tautomers and mixtures thereof also form an aspect of the present invention. Enantiomerically pure forms are particularly desired.

In one embodiment, the compounds of the invention wherein R² represents CH₂ possess the (S)-configuration at the 2-position of the pyrrolidine ring.

Thus in one embodiment, there is provided a compound of formula (Ic), or a to pharmaceutically acceptable salt thereof,

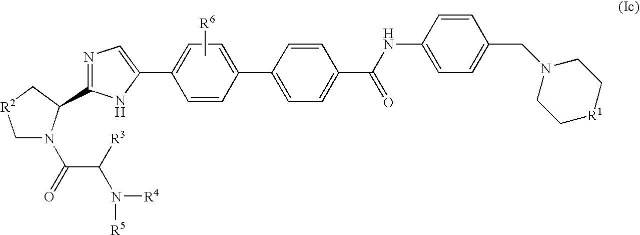

wherein R¹; R²; R³; R⁴; R⁵; R⁶; R⁷; R⁸; and R⁹ are as defined for formula (I).

In yet another embodiment, there is provided a compound of formula (Id), or a pharmaceutically acceptable salt thereof,

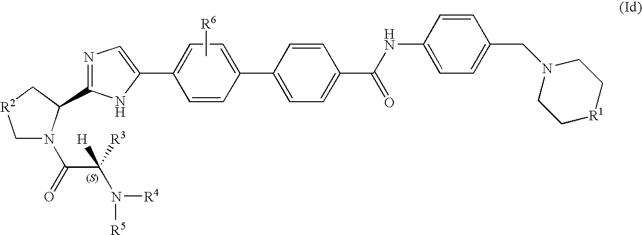

wherein R¹; R²; R³; R⁴; R⁵; R⁶; R⁷; R⁸; and R⁹ are as defined for formula (I) with the proviso that R³ is other than H.

Reference herein to a compound of formula (I) also includes within its meaning compounds of formulae (Ia), (Ib), (Ic) and (Id).

Compounds of formulae (I) may exist in crystalline form and exhibit polymorphism. It will be understood that the invention encompasses the use of all polymorphic forms of the compounds of formulae (I). Thus, in one embodiment of the present invention, there is provided a compound of formulae (I) in crystalline form.

The present invention further provides a process for the preparation of a compound of formula (I) or a pharmaceutically acceptable salt thereof as defined above which comprises,

(a) reacting a compound of formula (II)

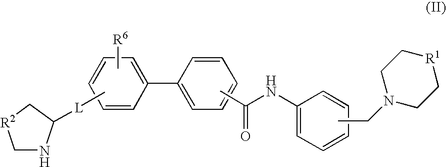

wherein L; R¹; R²; and R⁶ are as defined for formula (I),
with a compound of formula (III)

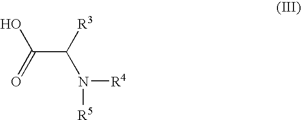

wherein R³, R⁴ and R⁵ are as defined in formula (I); or
(b) reacting a compound of formula (IV)

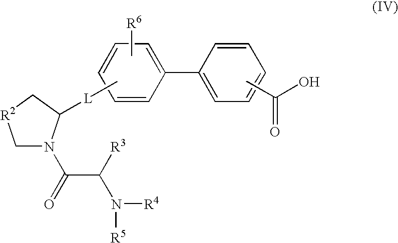

wherein L, R², R³, R⁴, R⁵ and R⁶ are as defined in formula (I),
with a compound of formula (V)

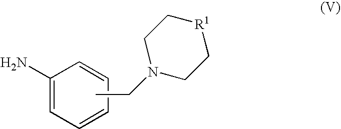

wherein R¹ is as defined in formula (I); or
(c) reacting together compounds of formulae (VI) and (VII)

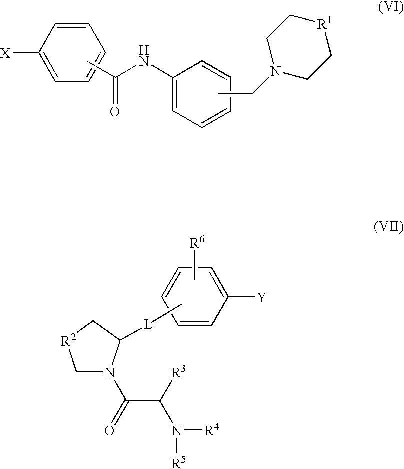

wherein L, R¹, R², R³, R⁴, R⁵ and R⁶ are as defined in formula (I) and either X represents halogen and Y represents —B(OH)₂ or an ester thereof; or Y represents halogen and X represents —B(OH)₂ or an ester thereof;
and optionally after (a), (b) or (c) carrying out one or more of the following:
converting the compound obtained to a further compound of the invention
forming a pharmaceutically acceptable salt of the compound.

In processes (a) and (b), the amide coupling reactions may be carried out by reaction of the amine with a carboxylic acid (or an acid chloride thereof) and a suitable coupling reagent such as HATU, HBTU or EDAC/HOBT, typically in the presence of a suitable base. Such processes are well known in the literature and will be readily apparent to the skilled man.

In process (c), the Suzuki type coupling may be effected by known methods, for is example, using cesium carbonate and a palladium catalyst in a suitable solvent such as DMF and at a suitable temperature.

Specific processes for the preparation of compounds of formula (I) are disclosed within the Examples section of the present specification. Such processes form an aspect of the present invention.

The necessary starting materials are either commercially available, are known in the literature or may be prepared using known techniques. Specific processes for the preparation of certain key starting materials are disclosed within the Examples section of the present specification and such processes form an aspect of the present invention.

Compounds of formula (I) can be converted into further compounds of formula (I) using standard procedures.

Certain intermediates may be novel. Such novel intermediates form another aspect of the invention.

It will be appreciated by those skilled in the art that in the processes of the present invention certain functional groups such as hydroxyl or amino or carboxyl groups may need to be protected by protecting groups. Thus, the preparation of the compounds of formula (I) may involve, at an appropriate stage, the addition and/or removal of one or more protecting groups.

The protection and deprotection of functional groups is described in Protective Groups in Organic Chemistry', edited by J. W. F. McOmie, Plenum Press (1973) and Protective Groups in Organic Synthesis', 3^{rd} edition, T. W. Greene and P. G. M. Wuts, Wiley-Interscience (1999).

The compounds of formula (I) above may be converted to a pharmaceutically is acceptable salt thereof, preferably an acid addition salt such as a hydrochloride, hydrobromide, sulphate, phosphate, acetate, fumarate, maleate, tartrate, lactate, citrate, pyruvate, succinate, oxalate, methanesulphonate or p-toluenesulphonate.

The compounds of formula (I) and their pharmaceutically acceptable salts have activity as pharmaceuticals, in particular as antiviral agents and especially as agents for the treatment of Flaviviridae infections.

More particularly, the compounds of formulae (I) and their pharmaceutically acceptable salts may be used in the treatment of hepatitis C virus.

Thus, the present invention provides a compound of formula (I) or a pharmaceutically-acceptable salt thereof as hereinbefore defined for use in therapy.

The present invention further provides a compound of formula (I) or a pharmaceutically-acceptable salt thereof as hereinbefore defined for use as a medicament.

In a further aspect, the present invention provides the use of a compound of formula (I) or a pharmaceutically acceptable salt thereof as hereinbefore defined in the manufacture of a medicament for use in therapy.

In a further aspect, the present invention provides the use of a compound of formula (I) or a pharmaceutically acceptable salt thereof as hereinbefore defined in the manufacture of a medicament for the treatment of hepatitis C virus.

In a further aspect, the present invention provides a compound of formula (I) or a pharmaceutically acceptable salt thereof as hereinbefore defined for the treatment of hepatitis C virus.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

The invention also provides a method of treating, or reducing the risk of, hepatitis C virus which comprises administering to a patient (for example a warm-blooded animal, is such as man) in need thereof a therapeutically effective amount of a compound of formula (I) or a pharmaceutically acceptable salt thereof as hereinbefore defined.

In a further aspect, the present invention provides the use of a compound of formula (I) or a pharmaceutically acceptable salt thereof as hereinbefore defined in the manufacture of a medicament for the treatment of Flaviviridae infections.

In a further aspect, the present invention provides a compound of formula (I) or a pharmaceutically acceptable salt thereof as hereinbefore defined for the treatment of Flaviviridae infections.

The invention also provides a method of treating, or reducing the risk of, Flaviviridae infections which comprises administering to a patient (for example a warm-blooded animal, such as man) in need thereof a therapeutically effective amount of a compound of formula (I) or a pharmaceutically acceptable salt thereof as hereinbefore defined.

For the above-mentioned therapeutic uses the dosage administered will, of course, vary with the compound employed, the mode of administration, the treatment desired and the disorder indicated. The daily dosage of the compound of the invention may be in the range from 0.01 mg/kg to 100 mg/kg. A unit dose form such as a tablet or a capsule will usually contain 1-250 mg of active ingredient. For example, a compound of formula (I), such as methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate, could be administered to a human patient at a dose of between 100-250 mg either once a day or twice a day.

The compounds of formula (I) and pharmaceutically acceptable salts thereof may be used on their own but will generally be administered in the form of a pharmaceutical composition in which the formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 is to 50% w, of active ingredient, all percentages by weight being based on total composition.

The present invention also provides a pharmaceutical composition comprising a compound of formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of formula (I) or a pharmaceutically acceptable salt thereof as hereinbefore defined with a pharmaceutically acceptable adjuvant, diluent or carrier.

The compounds of the invention may be administered in a variety of dosage forms. Thus, they can be administered orally, for example as tablets, troches, lozenges, aqueous or oily suspensions, dispersible powders or granules. The compounds of the invention may also be administered parenterally, whether subcutaneously, intravenously, intramuscularly, intrasternally, transdermally or by infusion techniques. The compounds may also be administered as suppositories.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the is active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

The compounds of the invention may also be administered in conjunction with other compounds used for the treatment of viral infections.

Thus, the invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of a viral infection, particularly Flaviviridae infections, particularly infection by hepatitis C virus.

The compounds of the invention may be administered in conjunction with one or more further active ingredients that are selected from:

(a) a HCV protease inhibitor, for example BI-1335, TMC435350, MK70009, ITMN-191, BILN-2061, VX-950, BILN-2065, BMS-605339, VX-500 and SCH 503034;
(b) a HCV polymerase inhibitor, for example R-7128, MK-0608, VCH759, PF-868554, GS9190, NM283, valopicitabine, PSI-6130, XTL-2125, NM-107, R7128 (R4048), GSK625433, R803, R-1626, BILB-1941, HCV-796, JTK-109 and JTK-003, benzimidazole derivatives, benzo-1,2,4-thiadiazine derivatives and phenylalanine derivatives;
(c) a HCV helicase inhibitor;
(d) an immunomodulatory agent, for example α-, β-, and γ-interferons such as IFN-α 2b, IFN-α 2ba, consensus IFN-α (infergen), feron, reaferon, intermax α, IFN-β, infergen+actimmune, IFN-omega with DUROS, albuferon, locteron, Rebif, Oral IFN-α, IFN-α 2b XL, AVI-005, pegylated-infergen, pegylated derivatized interferon-α compounds such as pegylated IFN-α 2b, pegylated IFN-α 2a, pegylated IFN-β, compounds that stimulate the synthesis of interferon in cells, interleukins, Toll like receptor (TLR) agonists, compounds that enhance the development of type 1 helper T cell response and thymosin;
(e) other antiviral agents, for example ribavirin, ribavirin analogs such as rebetol, copegus and viramidine (taribavirin), amantadine, and telbivudine, inhibitors of internal ribosome is entry, alpha-glucosidase 1 inhibitors such as MX-3253 (celgosivir) and UT-231B, hepatoprotectants such as IDN-6556, ME-3738, LB-84451 and MitoQ, broad-spectrum viral inhibitors, such as IMPDH inhibitors (e.g., mycophenolic acid and derivatives thereof, and VX-497, VX-148, and/or VX-944);
(f) a HCV NS5a inhibitor such as A-831 and A-689 or BMS-790052; and
(g) other drugs for treating HCV such as zadaxin, nitazoxanide, BIVN-401 (virostat), PYN-17 (altirex), KPE02003002, actilon (CPG-10101), KRN-7000, civacir, GI-5005, ANA-975, XTL-6865, ANA-971, NOV-205, tarvacin, EHC-18, NIM811, DEBIO-025, SCY635, VGX-410C, EMZ-702, AVI 4065, Bavituximab, and Oglufanide.

In particular the compounds of the invention may be administered in conjunction with one or more further active ingredients that are selected from:

a) a HCV protease inhibitor;

b) a HCV polymerase inhibitor;

c) a HCV helicase inhibitor;

d) an interferon; and

e) ribavirin.

According to this aspect of the invention there is provided a combination suitable for use in the treatment of hepatitis C virus infection, comprising:
a compound of formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof;
a HCV protease inhibitor, for example VX950, and/or a HCV polymerase inhibitor, for example HCV-796;
an interferon, for example pegylated IFN-α 2a α-interferon; and
ribavarin.

Therefore in a further aspect of the invention there is provided a compound of formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof, in combination with:
a HCV protease inhibitor, for example VX950, and/or a HCV polymerase inhibitor, for example HCV-796;
an interferon, for example pegylated IFN-α 2a α-interferon; and ribavarin.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof, in combination with:
a HCV protease inhibitor, for example VX950, and/or a HCV polymerase inhibitor, for example HCV-796;
an interferon, for example pegylated IFN-α 2a α-interferon; and ribavirin;
   and in association with a pharmaceutically acceptable diluent or carrier.

According to a further aspect of the invention there is provided a pharmaceutical composition which comprises a compound of formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof, in combination with:
a HCV protease inhibitor, for example VX950, and/or a HCV polymerase inhibitor, for example HCV-796;
an interferon, for example pegylated IFN-α 2a α-interferon; and
ribavirin;
   and in association with a pharmaceutically acceptable diluent or carrier for use in the is treatment of hepatitis C virus infection.

According to another feature of the invention there is provided the use of a compound of the formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof, in combination with:
a HCV protease inhibitor, for example VX950, and/or a HCV polymerase inhibitor, for example HCV-796;
an interferon, for example pegylated IFN-α 2a α-interferon; and
ribavirin;
   in the manufacture of a medicament for use in the treatment of hepatitis C virus infection.

According to another feature of the invention there is provided a compound of the formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof, in combination with:
a HCV protease inhibitor, for example VX950, and/or a HCV polymerase inhibitor, for example HCV-796;
an interferon, for example pegylated IFN-α 2a α-interferon; and
ribavirin;
   for use in the treatment of hepatitis C virus infection.

Therefore in an additional feature of the invention, there is provided a method for the treatment of hepatitis C virus infection in a patient (for example a warm-blooded animal, such as man) in need of such treatment which comprises administering to said animal an effective amount of a compound of formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof, in combination with:
a HCV protease inhibitor, for example VX950, and/or a HCV polymerase inhibitor, for example HCV-796;
an interferon, for example pegylated IFN-α 2a α-interferon; and
ribavirin.

According to a further aspect of the present invention there is provided a kit comprising a compound of formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof, in combination with:
a HCV protease inhibitor, for example VX950, and/or a HCV polymerase inhibitor, for example HCV-796;
an interferon, for example pegylated IFN-α 2a α-interferon; and
ribavirin.

According to a further aspect of the present invention there is provided a kit comprising:

a) a compound of formula (I) as defined hereinbefore, for example methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate or a pharmaceutically acceptable salt thereof, in a first unit dosage form;
b) a HCV protease inhibitor, for example VX950, and/or a HCV polymerase inhibitor, for example HCV-796, in a second unit dosage form;
c) an interferon, for example pegylated IFN-α 2a α-interferon, in a third unit dosage form;
d) ribavirin, in a fourth unit dosage form; and
e) container means for containing said first, second, third and fourth dosage forms.

The present invention will now be further explained by reference to the following illustrative examples.

### General Methods

The following general methods were used unless otherwise stated in relation to a particular example below.

¹H NMR spectra were recorded on a Bruker 250 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Unless stated otherwise, starting materials were commercially available. All solvents and commercial reagents were of laboratory grade and were used as received.

The following methods were used for LC-MS analysis:

### Method 1

Liquid Chromatograph (LC): Agilent 1200 series, with PDA detector, scan range 190-400 nm
Mass spectrometer: Agilent MSD 6120 operating in electrospray ionisation mode with +ve/−ve ion switching.

### LC Conditions:

Mobile phase A: 0.1% formic acid/10 mM ammonium formate in water.
Mobile phase B: Acetonitrile.

### Gradient:

| Time (mins.) | % B |
|---|---|
| 0 | 5 |
| 4 | 95 |
| 4.9 | 95 |
| 5 | 5 |

Flow rate: 1.0 ml/min.
Column: Varian Pursuit Ultra 3 C18 50 mm×2.1 mm
Column temperature: 50° C.

### Method 2

Liquid Chromatograph: Waters Acquity HPLC, with PDA detector, (scan range 190-400 nm) and ELSD.
Mass spectrometer: Waters SQD operating in electrospray ionisation mode with +ve/−ve ion switching.

### LC Conditions

Mobile phase A: 0.1% ammonia in water
Mobile phase B: 0.1% ammonia in acetonitrile

### Gradient

| Time (mins) | % B |
|---|---|
| 1 | 5 |
| 0.2 | 5 |
| 4.5 | 95 |
| 6 | 95 |

Flow rate: 0.6 ml/min
Column: Waters Acquity HPLC BEH C18 50 mm×2.1 mm 1.7 um
Column temperature: 50° C.

The abbreviations or terms used in the Examples have the following meanings:
DCM: Dichloromethane
DIPEA: N,N-Di-isopropylethylamine
DME: 1,2-Dimethoxyethane
DMF: N,N-Dimethylformamide
DMSO: Dimethyl sulphoxide
HATU: O-(7-Azabenzotriazol-1-yl)-N,N,N′,N′-tetramethyluronium hexafluorophosphate
HBTU: O-(Benzotriazol-1-yl)-N,N,N′,N′-tetramethyluronium hexafluorophosphate
NMM: N-methyl morpholine
HOBT Hydroxybenzotriazole
EDAC 1-ethyl-3(3-dimethylaminopropyl)carbodiimide
THF Tetrahydrofuran
MTBE Methyl tert-butyl ether
Me Methyl
Et Ethyl
Ac Acetyl
Ph Phenyl
GC Gas chromatography
TFA 2,2,2-trifluoroacetic acid
h or hr hour
min minute
HPLC high pressure liquid chromatography

### Preparation of Starting Materials

The starting materials for the Examples are either: commercially available, readily prepared by published methods; or described below.

The acid intermediates used in the synthesis of the Examples are described in the table below.

| Acid | | | Examples for |
|---|---|---|---|
| No. | Structure | Name | which used |
| A1 | 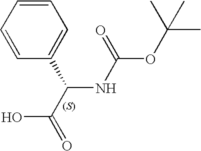 | (S)-2-((tert-butoxycarbonyl)amino)- 2-phenylacetic acid | 1, 48, 52 |
| A2 | 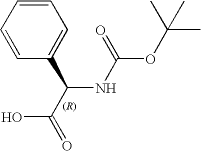 | (R)-2-((tert- butoxycarbonyl)amino)-2- phenylacetic acid | 2, 31 |
| A3 | 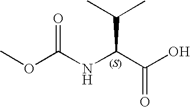 | (S)-2-(methoxycarbonylamino)-3- methylbutanoic acid | 3, 20, 28, 33, 40, 46, 50, 54 |
| A4 | 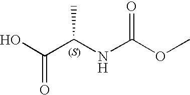 | (2S)-2- (methoxycarbonylamino)propanoic acid | 4, 21, 29, 34, 41 |
| A5 | 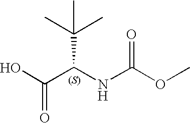 | (S)-2-((methoxycarbonyl)amino)- 3,3-dimethylbutanoic acid | 5, 22, 35 |
| A6 | 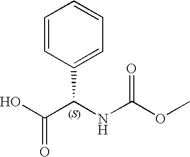 | (S)-2-((methoxycarbonyl)amino)-2- phenylacetic acid | 6, 23, 30, 36, 42, 44, 47, 51 |
| A7 | 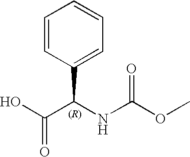 | (R)-2-((methoxycarbonyl)amino)-2- phenylacetic acid | 7, 24, 31, 37, 43 |
| A8 | 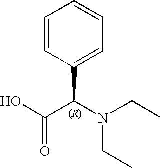 | (R)-2-(diethylamino)-2- phenylacetic acid | 8, 25, 32, 38, 44 |
| A9 | 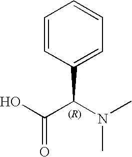 | (R)-2-(dimethylamino)-2- phenylacetic acid | 9, 26, 39, 45, 49, 53 |
| A10 | 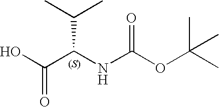 | (S)-2-((tert-butoxycarbonyl)amino)- 3-methylbutanoic acid | 10 |
| A11 | 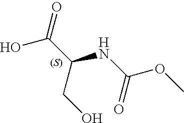 | (S)-3-hydroxy-2- ((methoxycarbonyl)amino) propanoic acid | 11 |
| A12 | 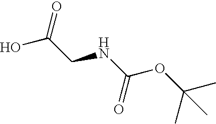 | 2-((tert- butoxycarbonyl)amino)acetic acid | 12 |
| A13 | 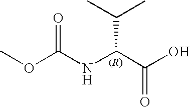 | (R)-2-((methoxycarbonyl)amino)-3- methylbutanoic acid | 13, 27 |
| A14 | 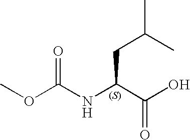 | (S)-2-((methoxycarbonyl)amino)-4- methylpentanoic acid | 14 |
| A15 | 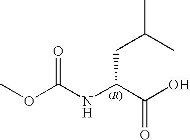 | (R)-2-((methoxycarbonyl)amino)-4- methylpentanoic acid | 15 |
| A16 | 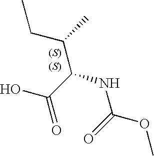 | (2S,3S)-2- ((methoxycarbonyl)amino)-3- methylpentanoic acid | 16 |
| A17 | 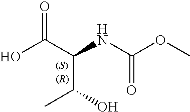 | (2S,3R)-3-hydroxy-2- ((methoxycarbonyl)amino)butanoic acid | 17 |
| A18 | 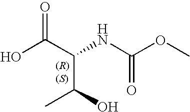 | (2R,3S)-3-hydroxy-2- ((methoxycarbonyl)amino)butanoic acid | 18 |
| A19 | 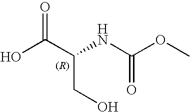 | (R)-3-hydroxy-2- ((methoxycarbonyl)amino) propanoic acid | 19 |

### Preparation of A11, A16 & A19

The appropriate amino acid, sodium carbonate (2 eq) and sodium hydroxide (aq, 1M, 1.05 eq) were placed in a 100 ml Rb flask and cooled to 5° C. Methyl chloroformate (1.08 eq) was added dropwise, stirred at 5° C. for 45 min then at ambient for 4 h. The RM was diluted with water, washed with DCM, and the aqueous phase cooled to 5° C. and acidified to pH1 by addition of conc.HCl. The volatiles were removed in vacuo and the residue taken up in MeOH/DCM, filtered and the organic phase concentrated to give the crude intermediates which were used crude in the subsequent coupling procedures.

The intermediates described may be combined to give the title compounds according to Scheme 1 wherein the synthesis of tert-butyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate is shown as an example.

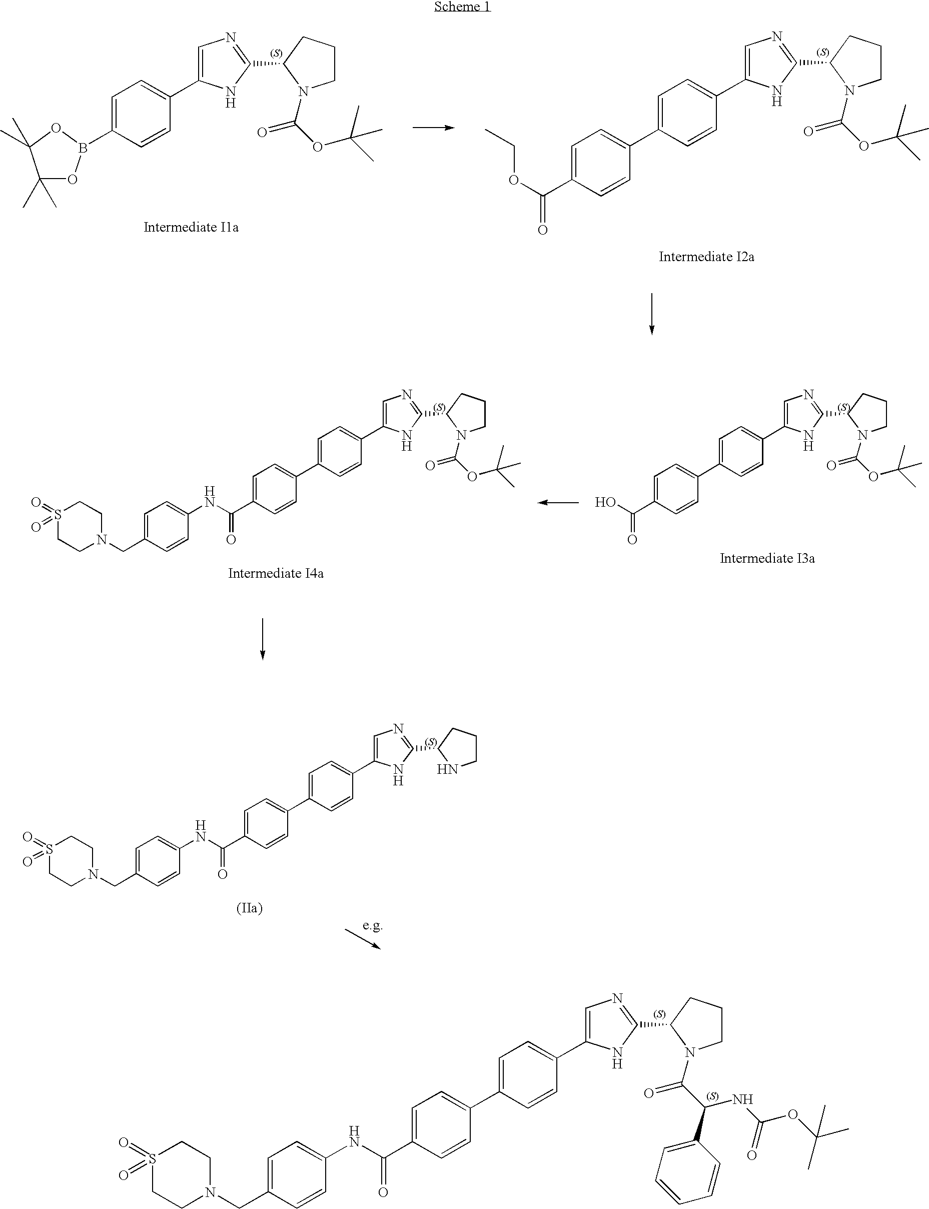

### Preparation of Scheme 1 Intermediates

### Preparation of tert-butyl (2S)-2-[4-[4-(4-ethoxycarbonylphenyl)phenyl]-1H-imidazol-2-yl]pyrrolidine-1-carboxylate (12a)

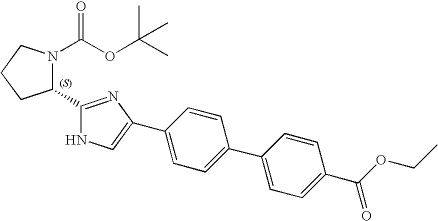

A mixture of tert-butyl (2S)-2-[4-[4-(4,4,5,5-tetramethyl-1,3,2-dioxaborolan-2-yl)phenyl]-1H-imidazol-2-yl]pyrrolidine-1-carboxylate (11a, Example 1c from WO 2008/021927) (4.3 g), ethyl bromobenzoate (1.76 ml), CsCO₃ (3.2 g) in degassed DME (30 ml) and water (15 ml) was treated with tetrakis(triphenylphosphine) palladium (0) (553 mg) and heated to 85° C. for 7 h. The reaction mixture was allowed to cool, concentrated, and the residue partitioned between water and ethyl acetate. The organic phase was concentrated onto a silica gel cartridge and purified by chromatography on silica gel. Gradient elution with petrol-ethyl acetate (4:1 to 1:1) over 35 mins gave a cream solid. (2.448 g).

LC-MS m/z (low cone voltage) 461;

¹H NMR (δ, d₆-DMSO) 1.14-1.4 (13H, m), 1.8-2.2 (4H, br m), 3.5 (1H, br s), 4.32 (2H, br q), 4.8 (1H, br m), 7.5-8.1 (m, 9H), d 11.9 (1H, br m).

### Preparation of 4-[4-[2-[(2S)-1-Tert-butoxycarbonylpyrrolidin-2-yl]-1H-imidazol-4-yl]phenyl]benzoic acid (13a)

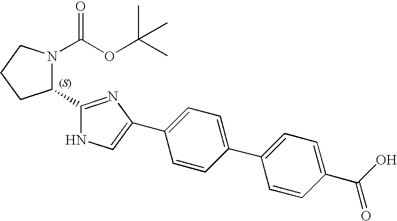

A suspension of tert-butyl (2S)-2-[4-[4-(4-ethoxycarbonylphenyl)phenyl]-1H-imidazol-2-yl]pyrrolidine-1-carboxylate (12a, 2.4 g) in ethanol (100 ml) and 2N NaOH (50 ml) was stirred at 20° C. for 18 h. The mixture was concentrated and cautiously neutralised to pH 6 with hydrochloric acid, then extracted into ethyl acetate (3×100 ml). The combined organic phases were dried and concentrated to a pale yellow solid (1.808 g).

LC-MS m/z (low cone voltage) 434;

¹H NMR (δ, d₆-DMSO) 1.2 (6H, s), 1.39 (3H, s), 1.9 (4H, m), 2.2 (2H, br m), 4.8 (2H, br m), 7.5 (1H, br s), 7.7-7.85 (6H, m), 8.0 (2H, d, J=8.5 Hz), d 11.9 (1H, br m).

### Preparation of tert-butyl (2S)-2-[4-[4-[4-[[4-[(1,1-dioxo-1,4-thiazinan-4-yl)ethyl]phenyl]carbamoyl]phenyl]phenyl]imidazol-2-yl]pyrrolidine-1-carboxylate (14a)

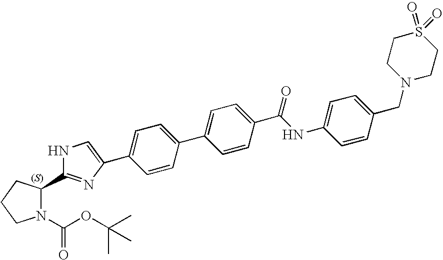

A solution of 4-[4-[2-[(2S)-1-tert-butoxycarbonylpyrrolidin-2-yl]-1H-imidazol-4-yl]ilphenyl]benzoic acid (13a, 1.8 g) and 4-[(1,1-dioxo-1,4-thiazinan-4-yl)methyl]aniline (1.05 eq.) in DMF (25 ml) was treated with HBTU (2.4 g) and N-methylmorpholine (1.4 ml) and allowed to stir for 2 days at 20° C. The mixture was concentrated and then partitioned between ethyl acetate and water. The dried organic phase was concentrated onto silica gel. Purification by column chromatography on silica with DCM/EtOH/NH₃ (200:8:1) to (50:8:1) gave partial purification. Further chromatography of the material on silica gel was carried out with ethyl acetate as eluent, giving a colourless solid (1 g).

LC-MS m/z (low cone voltage) 656;

¹H NMR (δ, d₆-DMSO) 1.14 (6H, s), 1.39 (3H, s), 1.78-2.32 (4H, b), 2.87-2.90 (4H, m), 3.05-3.15 (4H, m), 3.47-3.63 (3H, b+s), 4.73-4.90 (1H, b), 7.31 (2H, d, J=8.53 Hz), 7.56 (1H, b), 7.72-7.89 (8H, m), 8.05 (2H, d, J=8.53 Hz), 10.29 91H, s).

### Preparation of N-[4-[(1,1-dioxo-1,4-thiazinan-4-yl)methyl]phenyl]-4-[4-[2-[(2S)-pyrrolidin-2-yl]-1H-imidazol-5-yl]phenyl]benzamide (IIa)

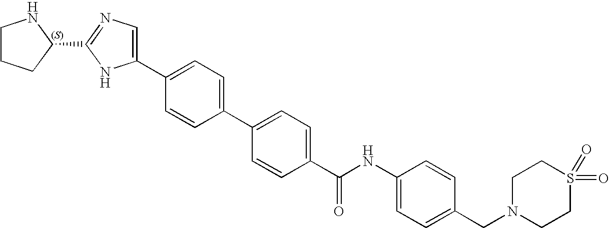

The title intermediate was prepared by the general method for Boc deprotection using tert-butyl (2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carboxylate (14a)

Pale yellow solid (84%)

### LC-MS m/z 556;

¹H NMR (δ, d₆-DMSO) 1.67-2.15 (4H, b), 2.81-2.92 (4H, m), 3.04 3.15 (4H, m), 3.31-3.40 (2H, b), 3.62 (2H, s), 4.02-4.23, 4.61-4.80 (1H, 2xb), 7.31 (2H, d, J=8.53 Hz), 7.40-7.58 (2H, b), 7.69-7.91 (8H, m), 8.04 (2H, d, J=8.53 Hz), 10.29-10.44 (1H, b)

The intermediates described may also be combined to give the title compounds according to Scheme 2 wherein the synthesis of tert-butyl (2S)-2-[4-[4-[4-[[4-[(1,1-dioxo-1,4-thiazinan-4-yl)methyl]phenyl]carbamoyl]phenyl]phenyl]imidazol-2-yl]pyrrolidine-1-carboxylate (14a) is shown as an example.

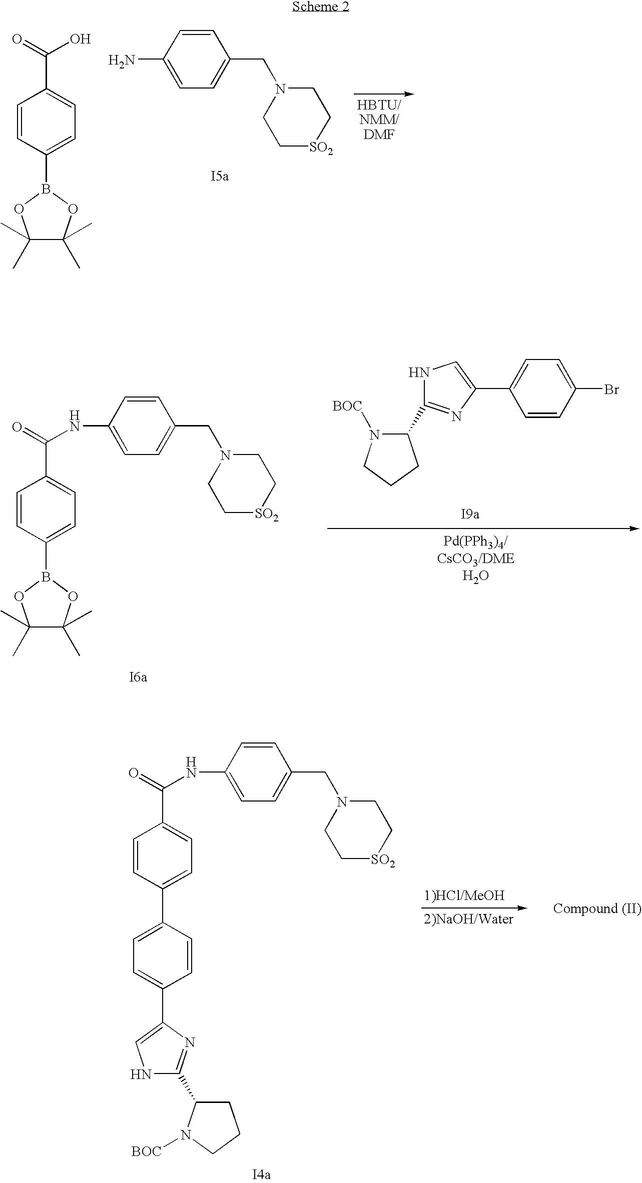

### Preparation of Intermediate 15a Analogue

### Preparation of 4-[(4-(propylsulfonyl)piperazin-1-yl)methyl]aniline (15b)

### 1-(Propylsulfonyl)piperazine

A cooled (0 C), stirred mixture of ethyl piperazine-1-carboxylate (8 g) and triethylamine (7 ml) in TBME (40 ml) was treated dropwise with propylsulfonyl chloride (7.13 g). After 90 mins the mixture was filtered and the collected material slurried in water, filtered again, and dried giving a colourless solid (9.54 g). A portion of this material (7 g) in ethanol (70 ml) and 4M sodium hydroxide (70 ml) was stirred and heated to 10° C. for 20 h. The ethanol was then evaporated and the aqueous residue extracted with THF and ethyl acetate. The combined, dried extracts were evaporated giving the title compound as a straw coloured oil (4.9 g)

¹H NMR (δ, d₆-DMSO) 0.75 (t, 3H, J=7.58 Hz) 1.45 (sextet, 2H, J=7.58 Hz) 2.43-2.54 (m, 4H) 2.69-2.84 (m+t, 6H) 2.95-3.15 (brs, >1H)

### 1-(4-nitrobenzyl)-4-(propylsulfonyl)piperazine

To a cooled (0 C) stirred mixture of 1-(propylsulfonyl)piperazine (4.9 g) and potassium carbonate (7.14 g) in DMF (35 ml) was added dropwise a solution of 4-nitrobenzyl bromide (5.59 g) in DMF (10 ml). The mixture was allowed to warm to room temperature and then stirred a further 1 h. This mixture was then added portionwise to stirred ice/water (800 ml) whereupon a solid was produced which was collected by filtration, and dried, yielding a colourless solid (7.95 g).

¹H NMR (δ, d₆-DMSO) 1.20 (t, 3H, J=7.58 Hz) 1.91 (sextet, 2H, J=7.58 Hz) 2.62-2.76 (m, 4H) 3.24 (t, 2H, J=7.58 Hz) 3.36-3.45 (m, 4H) 3.89 (s, 2H) 7.83 (d, 2H, J=8.21 Hz) 8.43 (d, 2H, J=8.12 Hz)

### 4-[(4-(Propylsulfonyl)piperazin-1-yl)methyl]aniline (15b)

A solution of 1-(4-nitrobenzyl)-4-(propylsulfonyl)piperazine (5.5 g) in methanol (250 ml) was hydrogenated over 5% platinum on carbon at atmospheric pressure. The mixture was then filtered through celite and the solvent evaporated giving the title compound as an off-white solid (5.11 g)

¹H NMR (δ, d₆-DMSO) 1.04 (t, 3H, J=7.58 Hz) 1.74 (sextet, 2H, J=7.58 Hz) 2.39-2.48 (m, 4H) 3.05 (t, 2H, J=7.58 Hz) 3.14-3.26 (m, 4H) 3.36 (s, 2H) 5.04 (s, 2H) 6.56 (d, 2H, J=8.21 Hz) 6.98 (d, 2H, J=8.21 Hz)

### Preparation of N-[4-[(1,1-dioxo-1,4-thiazinan-4-yl)methyl]phenyl]-4-(4,4,5,5-tetramethyl-1,3,2-dioxaborolan-2-yl)benzamide (16a)

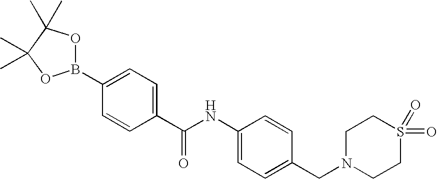

A solution of 4-((1,1-dioxo-1,4-thiazinan-4-yl)methyl)aniline (10.2 g, 42 mmol) and 4-(4,4,5,5-tetramethyl-1,3,2-dioxaborolan-2-yl)benzoic acid (10 g, 40 mmol) in dry DMF (70 ml) was treated with HBTU (23 g, 60 mmol) and NMM (13.2 ml, 120 mmol) and stirred at room temperature for 1.5 h. A thick precipitate formed. H₂O (200 ml) was added and the precipitate collected by filtration and dried in vacuo. Solid was dissolved in 20% MeOH/DCM (600 ml) and insoluble impurities removed by filtration. The mother liquor to was concentrated in vacuo and the yellow solid triturated with EtOAc (250 ml) to yield the title compound as a pale yellow solid (16.88 g, 89%)

LC-MS m/z 471;

¹H NMR (8, d₆-DMSO) 1.31 (12H, s), 2.86 (4H, m), 3.09 (4H, m), 3.62 (2H, s), 7.30 (2H, d, J=8.53 Hz), 7.74 (2H, d, J=8.53 Hz), 7.79 (2H, d, 8.53 Hz), 7.95 (2H, d, J=8.21 Hz), 10.31 (1H, s).

### Preparation of Intermediate 19a Anatomies

### Preparation of (S)-tert-butyl 3-(2-(4-bromophenyl)-2-oxoethylcarbamoyl)morpholine-4-carboxylate (18f)

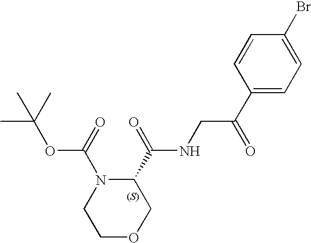

A mixture of 2-amino-1-(4-bromophenyl)ethanone hydrochloride (2 g) and (3S)-4-tert-butoxycarbonylmorpholine-3-carboxylic acid (1.84 g) in DMF (20 ml) was treated with HBTU (4.54 g) and N-methylmorpholine (4.03 ml) at 20 C for 18 h. The solvent was evaporated and the residue partitioned between water and DCM. The dried extracts were evaporated and then purified on silica gel. Elution with DCM:EtOH:NH3; 500:8:1 gave a pale yellow foam (2.4 g).

LC/MS m/z 328 (M-Boc)

1H NMR (δ, CDCl3) 1.45 (s, 9H), 3.15-3.84 (brm, 5H), 4.42-4.82 (brm, 4H), 6.95 (brs, 1H), 7.58 (d, 2H, J=8.84 Hz), 7.76 (d, 2H, J=8.84 Hz)

### Preparation of (S)-tert-butyl 2-(2-(4-bromophenyl)-2-oxoethylcarbamoyl)piperidine-1-carboxylate (18g)

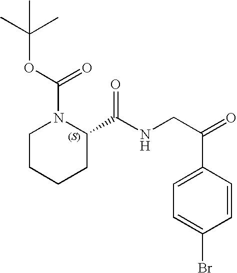

The title compound was produced using the same method as (S)-tert-butyl 3-(2-(4-bromophenyl)-2-oxoethylcarbamoyl)morpholine-4-carboxylate (18f) using (2S)-1-tert-butoxycarbonylpiperidine-2-carboxylic acid as starting material.

Pale yellow solid (3.12 g)

LC/MS m/z 426 (low cone voltage)

1H NMR (δ, d6 DMSO) 1.20-1.28 (m, 2H), 1.38 (s, 9H), 1.53 (m, 3H), 2.11 (d, 1H, J=12.95 Hz), 2.49 (m, 1H), 3.82 (d, 1H, J=13.58 Hz), 4.52-4.68 (m, 3H), 7.74 (d, 2H, J=8.53 Hz), 7.91 (d, 2H, J=8.84 Hz), 8.13 (brs, 1H)

### Preparation of tert-butyl (3R)-3-[4-(4-bromophenyl)-1H-imidazol-2-yl]morpholine-4-carboxylate (19f)

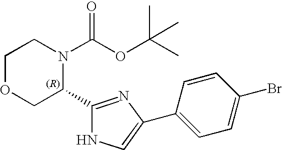

A mixture of (S)-tert-butyl 3-(2-(4-bromophenyl)-2-oxoethylcarbamoyl)morpholine-4-carboxylate (18f, 2.3 g) and ammonium acetate (4.1 g) was heated to 12° C. in toluene (40 ml) for 18 h. The mixture was allowed to cool and was evaporated. The residue was partitioned between aq. sodium bicarbonate and DCM. The dried extracts were evaporated and the residue purified on silica gel. Elution with DCM:EtOH:NH3; 400:8:1 gave a light brown solid (1.42 g)

LC/MS m/z 408, 410

1H NMR (δ, CDCl3) 1.54 (s, 9H), 3.11-3.24 (m, 1H), 3.63 (dt, 1H, J=2.84, 12.00 Hz), 3.80-3.91 (m, 2H), 3.98 (dd, 1H, J=3.16, 8.53 Hz), 4.61 (d, 1H, J=12.00 Hz), 5.25 (d, 1H, J=3.16 Hz), 7.29 (s, 1H), 7.50 (d, 2H, J=8.53 Hz), 7.60 (d, 2H, J=8.53 Hz)

### Preparation of tert-butyl (2S)-2-[4-(4-bromophenyl)-1H-imidazol-2-yl]piperidine-1-carboxylate (19g)

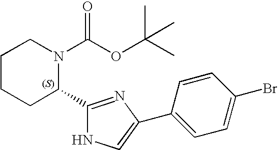

A mixture of (S)-tert-butyl 2-(2-(4-bromophenyl)-2-oxoethylcarbamoyl)piperidine-1-carboxylate (18 g, 3.1 g) and ammonium acetate (5.5 g) was heated to 14° C. in xylene (50 ml) for 18 h. The mixture was allowed to cool and was evaporated. The residue was is partitioned between aq. sodium bicarbonate and DCM. The dried extracts were evaporated and the residue purified on silica gel. Elution with DCM:EtOH:NH3; 400:8:1 gave a brown foam (2.39 g)

LC/MS m/z 406, 408 (low cone voltage)

1H NMR (δ, CDCl3) 1.24-1.49 (m, 2H), 1.52 (s, 9H), 1.55-1.88 (m, 4H), 2.52-2.62 (m, 1H), 2.72-2.85 (m, 1H), 3.99 (m, 1H), 5.41 (m, 1H), 7.25 (s, 1H), 7.50 (d, 2H, J=8.53 Hz), 7.60 (brd, 2H, J=8.53 Hz)

### Preparation of 2-bromo-1-(4-bromo-3-methyl-phenyl)ethanone (17h)

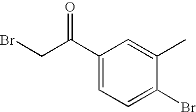

To a 100 ml round bottom flask was added 1-(4-bromo-3-methyl-phenyl)ethanone (2 g, 9.38 mmol), dry dioxan (50 ml) and CuBr₂ (4.2 g, 18.70 mmol). Mixture was stirred at 100° C. under nitrogen for 3 hours. The mixture was cooled and filtered and the filtrate was concentrated to dryness to afford a green oil which was loaded onto a silica column and eluted with DCM. This gave the title compound as a white solid 1.80 g (66%)

¹H NMR (δ, d₆-DMSO) 2.41 (3H, s), 4.91 (2H, s), 7.65-7.86 (2H, m), 7.92-8.04 (1H, m)

### Preparation of O2-[2-(4-bromo-3-methyl-phenyl)-2-oxo-ethyl]O1-tert-butyl (2S)-pyrrolidine-1,2-dicarboxylate (18h)

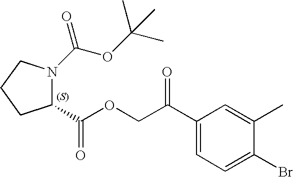

To a 100 ml round bottom flask was added 2-bromo-1-(4-bromo-3-methyl-phenyl)ethanone (17 h, 1.80 g, 6 mmol), CH₃CN (DRY 40 ml), and (S)-1-(tert-butoxycarbonyl)pyrrolidine-2-carboxylic acid (1.34 g, 6.22 mmol) and mixture was stirred at room temperature for 15 minutes under nitrogen. DIPEA (0.81 g, 6.28 mmol) was added over a period of 10 minutes and then left to stir overnight at room temperature. The mixture was concentrated to dryness and the crude product was put on a silica column and eluted with 2.5% MeOH:DCM to give the desired compound 2.5 g (100%) as a white solid.

LC-MS m/z 326 (M-100)

¹H NMR (δ, d₆-DMSO) 1.34 (6H, s), 1.38 (3H, s), 1.78-1.95 (2H, m), 2.04-2.34 (2H, b), 2.42 (3H, s), 4.27-4.35 (1H, m), 5.38-5.62 (2H, m), 7.66-7.78 (2H, m), 7.96 (1H, s)

### Preparation of tert-butyl (2S)-2-[4-(4-bromo-3-methyl-phenyl)-1H-imidazol-2-yl]pyrrolidine-1-carboxylate (19h)

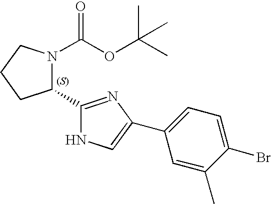

To a 250 ml round bottom flask was added O2-[2-(4-bromo-3-methyl-phenyl)-2-oxo-ethyl]O1-tert-butyl (2S)-pyrrolidine 1,2-dicarboxylate (18 h, 2.5 g, 5.86 mmol), NH₄OAc (4.50 g, 58.6 mmol) and toluene (DRY 60 ml) and mixture was stirred at 120° C. for 18 hrs, under nitrogen. The mixture was cooled and concentrated to dryness and the crude product was put on a silica column and eluted with 2.5% MeOH:DCM to give the title compound 2.30 g (96%) as a white solid

LC-MS m/z 406, 404

¹H NMR (8, d₆-DMSO) 1.13 (6H, s), 1.38 (3H, s), 1.71-2.28 (4H, m), 2.34 (3H, s), 3.36-3.78 (2H, m), 4.68-4.87 (1H, b), 7.47 (2H, s), 7.71 (1H, s), 11.82-12.29 (1H, b)

### Preparation of Intermediate 14a Analogues

### General Procedure for Suzuki Reactions

A solution of the appropriate aryl bromide (1.04 eq), aryl boronate (1 eq) and cesium carbonate (3.4 eq) in DME and H₂O (3:1) was heated to 85° C. under N₂ then treated with Pd(PPh₃)₄ (5 mol %). Heating at 85° C. was continued for 18 hrs then the mixture was cooled to room temperature and partitioned between DCM and H₂O. The organic extract was dried (MgSO₄) and concentrated onto silica gel before purification (silica gel column, is eluting with a gradient of 0-100% 200:8:1 DCM:EtOH:NH₃/DCM) to yield the desired compound.

### Preparation of tert-butyl (2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carboxylate (14a)

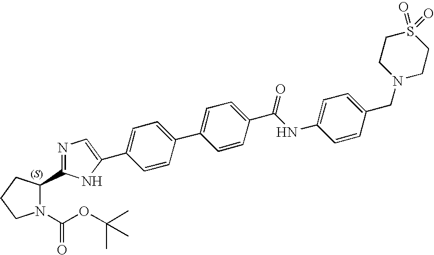

The title intermediate was prepared by general method for Suzuki reaction using tert-butyl (2S)-2-[5-(4-bromophenyl)-1H-imidazol-2-yl]pyrrolidine-1-carboxylate (19a) and N-[4-[(1,1-dioxo-1,4-thiazinan-4-yl)methyl]phenyl]-4-(4,4,5,5-tetramethyl-1,3,2-dioxaborolan-2-yl)benzamide (16a)

Pale yellow solid (5.24 g, 75%)

LC-MS m/z 656;

¹H NMR (δ, d₆-DMSO) 1.14 (6H, s), 1.39 (3H, s), 1.78-2.32 (4H, b), 2.87-2.90 (4H, m), 3.05-3.15 (4H, m), 3.47-3.63 (3H, b+s), 4.73-4.90 (1H, b), 7.31 ((2H, d, J=8.53 Hz), 7.56 (1H, b), 7.72-7.89 (8H, m), 8.05 (2H, d, J=8.53 Hz), 10.29 (1H, s), 11.83-11.99 (1H, b).

An alternative method for the preparation of compound 14a is as follows: 4-[(1,1-dioxo-1,4-thiazinan-4-yl)methyl]aniline (46 g) and 4-(4,4,5,5-tetramethyl-1,3,2-dioxaborolan-2-yl)benzoic acid (45.24 g) in dry DMF (300 ml) was treated with HBTU (103.6 g) and N-methyl morpholine (55.35 g, 60 ml) and stirred at room temperature overnight. The thick reaction mixture was diluted with water (˜1200 ml) and filtered, washed with water (3×300 ml) and dried in vacuo for 3 days. The resulting solid was slurried with methanol/dichloromethane (1:4, 500 ml) and filtered. This slurrying was repeated twice more and the collected filtrates concentrated to dryness. The residue was slurried in diethyl ether, filtered, washed with further diethyl ether and dried by suction then in vacuo to give the crude boronate (16a, 60.65 g, ˜75% pure). A portion of this material (47.5 g, 0.1 mol), (2S)-2-[5-(4-bromophenyl)-1H-imidazol-2-yl]-N-tert-butyl-pyrrolidine-1-carboxamide (Example 1b from WO 2008/021927, 39.6 g, 0.1 mol), and CsCO₃ (112.2 g) in DME (550 ml) and water (300 ml) was heated to 85° and treated with Pd(PPh₃)₄ (5.6 g). After 4 hours at 85°, reaction was complete so allowed to cool to room temperature overnight. The reaction mixture was separated and the organics washed with brine, separated and dried (MgSO₄). After concentrating to dryness, the resulting solid was slurried in acetone, filtered and washed with acetone (×2) then diethyl ether and dried in vacuo to give the title compound (14a, 47.05 g, 71%).

The following intermediates were prepared by the general procedure for Suzuki reaction using the appropriate aryl boronate and aryl bromide.

| | | | LC | |
|---|---|---|---|---|
| Name | Structure | Yield | MS | NMR |
| tert-Butyl (2R)- 2-[5-[4-[4-[[4- [(1,1-dioxo-1,4- thiazinan-4- yl)methyl]phenyl] carbamoyl]phenyl] phenyl]-1H- imidazol-2- yl]pyrrolidene- 1-carboxylate (I4b) | 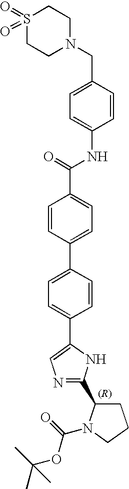 | Yellow foam (1.52 g, 59%) | m/z 656 | 1.15 (6H, s), 1.39 (3H, s), 1.78-2.32 (4H, b), 2.87- 2.90 (4H, m), 3.05-3.15 (4H, m), 3.47-3.63 (3H, b + s), 4.73-4.90 (1H, m), 7.31 (2H, d, J = 8.53 Hz), 7.56 (1H, b), 7.72-7.89 (8H, m), 8.05 (2H, d, J = 8.21 Hz), 10.29 (1H, s), 11.91 (1H, s) |
| tert-butyl (2S)- 2-[5-[4-[3-[[4- [(1,1-dioxo-1,4- thiazinan-4- yl)methyl]phenyl] carbamoyl]phenyl] phenyl]-1H- imidazol-2- yl]pyrrolidene- 1-carboxylate (I4c) | 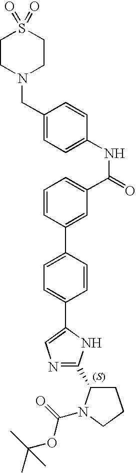 | White solid (1.0 g, 72%) | m/z 656 | 1.15 (6H, s), 1.39 (3H, s), 1.75-2.32 (4H, b), 2.85- 2.88 (4H, m), 3.07-3.13 (4H, m), 3.45-3.64 (3H, b + s), 4.73-4.90 (1H, b), 7.26-7.36 (3H, d + m, J = 8.53 Hz), 7.54-7.67 (2H, m), 7.70-7.81 (4H, m), 7.83-7.93 (3H, m), 8.22 (1H, s), 10.35 (1H, s), 11.84-11.97 (1H, b) |
| tert-Butyl (2S)- 2-[5-[3-[4-[[4- [(1,1-dioxo-1,4- thiazinan-4- yl)methyl]phenyl] carbamoyl]phenyl] phenyl]-1H- imidazol-2- yl]pyrrolidine- 1-carboxylate (I4d) | 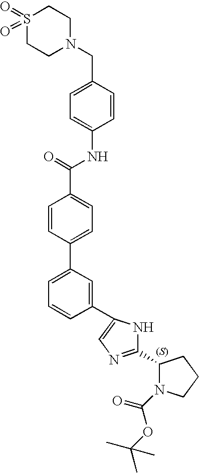 | White solid (1.81 g, 71%) | m/z 656 | 1.16 (6H, s), 1.39 (3H, s), 1.78-2.32 (4H, b), 2.86- 2.90 (4H, m), 3.05-3.15 (4H, m), 3.48-3.63 (3H, b + s), 4.70-4.88 (1H, b), 7.31 ((2H, d, J = 8.53 Hz), 7.83- 7.94 (3H, m), 7.99-8.10 (1H, m), 8.06 (2H, d, J = 8.53 Hz), 10.30 (1H, s), 11.83-12.01 (1H, b) |
| tert-Butyl (2S)- 2-[5-[4-[4-[[4- [(4- propylsulfonyl- piperazin-1- yl)methyl]phenyl] carbamoyl]phenyl] phenyl]-1H- imidazol-2- yl]pyrrolidine- 1-carboxylate (I4e) | 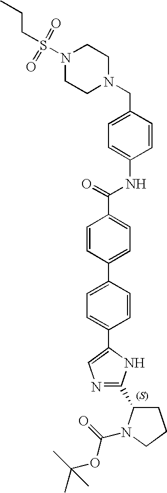 | Pale yellow solid (1.01 g, 75%) | m/z 713 | 0.97 (3H, t, J = 7.4 Hz), 1.15 (6H, s), 1.39 (3H, s), 1.60-1.75 (m, 2H), 1.78- 2.30 (4H, b), 2.40-2.44 (4H, m), 2.96-3.03 (2H, m), 3.10-3.20 (4H, m), 3.35-3.57 (3H, b + s), 4.73-4.89 (1H, b), 7.28 (2H, d, J = 8.53 Hz), 7.57 (1H, b), 7.69-7.94 (8H, m), 8.04 (2H, d, J = 8.53 Hz), 10.27 (1H, s), 11.85-11.98 (1H, b) |
| tert-butyl (2S)- 2-[5-[4-[4-[[4- [(1,1-dioxo-1,4- thiazinan-4- yl)methyl]phenyl] carbamoyl]phenyl]-3- methyl-phenyl]- 1H-imidazol-2- yl]pyrrolidine- 1-carboxylate (I4h) | 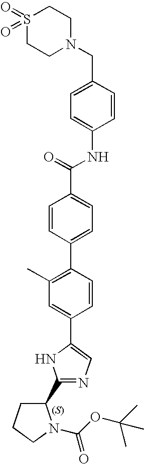 | Off white solid (35%) | m/z 670 | ¹H NMR (δ, d₆-DMSO) 1.05 (6H, s), 1.14 (6H, s), 1.31 (3H, s), 1.38 (3H, s), 1.67-2.11 (3H, b), 2.14- 2.35 (4H, s + b), 2.80-2.92 (4H, b), 3.02-3.20 (6H, b), 3.48-3.69 (3H, s + b), 3.94 (1H, s), 4.72-4.92 (0.75H, b), 7.17-8.08 (12H, m), 10.30 (1H, s), 11.87-12.03 (1H, b) |

### Preparation of tert-butyl (3R)-3-[5-[4-[4-[[4-[(1,1-dioxo-1,4-thiazinan-4-yl)methyl]phenyl]carbamoyl]phenyl]phenyl]-1H-imidazol-2-yl]morpholine-4-carboxylate (14f)

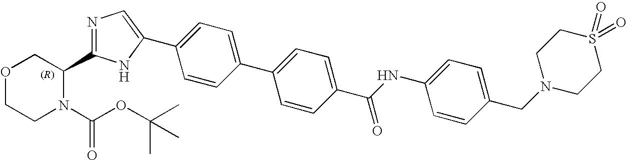

A mixture of tert-butyl (3R)-3-[4-(4-bromophenyl)-1H-imidazol-2-yl]morpholine-4-carboxylate (19f, 1 g) and N-[4-[(1,1-dioxo-1,4-thiazinan-4-yl)methyl]phenyl]-4-(4,4,5,5-tetramethyl-1,3,2-dioxaborolan-2-yl)benzamide (16a, 1.15 g) in 1:2 water:DME (30 ml) was heated to 10° C. in the presence of cesium carbonate (1.19 g) and to tetrakis(triphenylphosphine)palladium⁰ (120 mg) under nitrogen for 18 h. The mixture was cooled and then partitioned between water and DCM. The dried extract was evaporated and the residue purified on silica gel. Elution with DCM:EtOH:NH3; 300:8:1 gave a cream solid (556 mg)

LC/MS m/z 673

1H NMR (δ, d6 DMSO) 1.05 (m), 1.34-1.44 (m), 2.82-2.90 (m), 3.05-3.14 (m), 3.32 (m), 3.36-3.48 (m), 3.62-3.84 (brs+m), 4.20 (s+m), 4.26-4.36 (m), 5.00 (brs), 7.27-7.35 (m), 7.65 (m), 7.72-7.80 (m), 7.82-7.91 (m), 8.02-8.11 (m), 10.28 (brs)

### Preparation of tert-Butyl (2S)-2-[5-[4-[4-[[4-[(1,1-dioxo-1,4-thiazinan-4-yl)methyl]phenyl]carbamoyl]phenyl]phenyl]-1H-imidazol-2-yl]piperidine-1-carboxylate (14g)

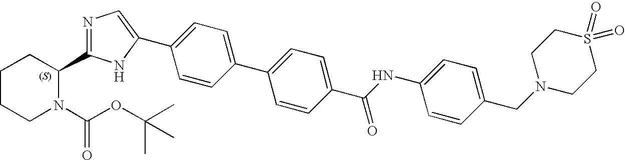

The title compound was produced by the same method as tert-butyl (3R)-3-[5-[4-[4-[[4-[(1,1-dioxo-1,4-thiazinan-4-yl)methyl]phenyl]carbamoyl]phenyl]phenyl]-1H-imidazol-2-yl]morpholine-4-carboxylate (16a) using tert-butyl (2S)-2-[4-(4-bromophenyl)-1H-imidazol-2-yl]piperidine-1-carboxylate (19g).

Pale yellow foam (1.21 g)

LC/MS m/z 670

1H NMR (δ, d6 DMSO) 1.35-1.64 (m+s, 14H), 2.16-2.24 (m, 1H), 2.82-2.89 (m, 4H), 3.06-3.16 (m, 4H), 3.36-3.48 (m, 1H), 3.63 (s, 2H), 3.85-3.92 (m, 1H), 5.28 (br, 1H), 7.30 (d, 2H, J=8.53 Hz), 7.62 (brs, 1H), 7.72-7.80 (m, 4H), 7.82-7.91 (m, 4H), 8.04 (d, 2H, J=8.53 Hz), 10.28 (s, 1H), 11.93 (brs, 1H)

### General Procedure for Boc Deprotection

A mixture of Boc amine (1.2 mmol), TFA (5 ml) and DCM (50 ml) was stirred for 18 hr. The mixture was concentrated and azeotroped with MeOH and then partitioned between DCM (30 ml) and a solution of saturated K₂CO₃ (30 ml). A yellow solid precipitated at the interface of the two solutions, which was filtered off and dried under vacuum at 40° C. and used without any further purification

The following intermediates were prepared by the general procedure for Boc deprotection using the appropriate protected amine.

| Name | Structure | Yield | LCMS | NMR |
|---|---|---|---|---|
| N-[4-[(1,1- dioxo-1,4- thiazinan-4- yl)methyl]phenyl]- 4-[4-[2-[(2R)- pyrrolidin-2-yl]- 1H-imidazol-5- yl]phenyl]benzamide (IIb) | 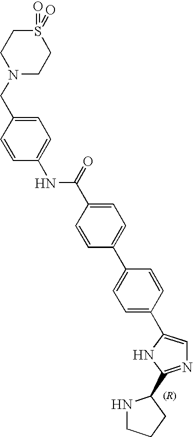 | Pale yellow solid (100%) | m/z 556 | 1.65-2.14 (4H, b), 2.50-2.65 (1H, b), 2.78-2.93 95H, m), 3.04-3.19 (5H, m), 3.63 (2H, s), 4.10- 4.20, 4.67-4.76 (1H, 2 × m), 7.29 (2H, d, J = 8.21 Hz), 7.39, 7.48 (1H, 2 × s), 7.65-7.90 (9H, m), 8.05 (2H, d, J = 8.21 Hz), 10.33- 10.76 (1H, b) |
| N-[4-[(1,1- dioxo-1,4- thiazinan-4- yl)methyl]phenyl]- 3-[4-[2-[(2S)- pyrrolidin-2-yl]- 1H-imidazol-5- yl]phenyl]benzamide (IIc) | 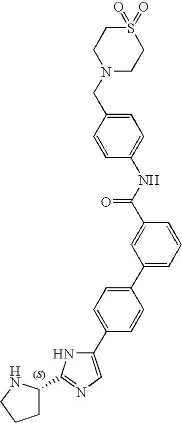 | Pale yellow solid (95%) | m/z 556 | 1.59-2.13 (4H, b), 2.75-2.91 (4H, m), 3.04-3.16 (4H, m), 3.24-3.43 (2H, m), 3.60 (3H, bs), 4.67- 4.74 (1H, m), 7.23 (3H, d, J = 8.53 Hz), 7.38 (1H, s), 7.52 (1H, t, 7.74 Hz), 7.65-7.84 (8H, m), 7.93 (1H, d, J = 7.89 Hz), 8.26 (1H, s) |
| N-[4-[(1,1- dioxo-1,4- thiazinan-4- yl)methyl]phenyl]- 4-[3-[2-[(2S)- pyrrolidin-2-yl]- 1H-imidazol-5- yl]phenyl]benzamide (IId) | 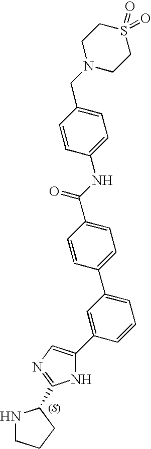 | Pale yellow solid (93%) | m/z 556 | 1.66-2.12 (4H, m), 2.79-2.90 (4H, m), 2.90-3.03 (1H, m), 3.04-3.19 (4H, m), 3.55-3.67 (3H, bs), 4.17 (1H, t, J = 6.95 Hz), 7.37 (2H, d, J = 8.53 Hz, 7.41- 7.60 (3H, m), 7.67- 7.95 (6H, m), 8.06 (3H, m), 10.32 (1H, s) |
| N-[4-[(4- propylsulfonyl- piperazin-1- yl)methyl]phenyl]- 4-[4-[2-[(2S)- pyrrolidin-2-yl]- 1H-imidazol-5- yl]phenyl]benzamide (IIe) | 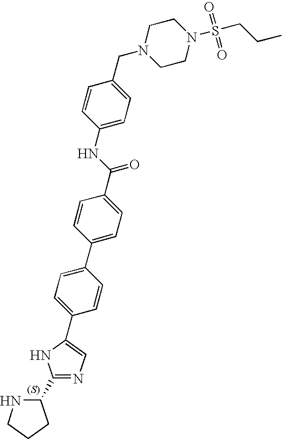 | Pale yellow solid (98%) | m/z 613 | 0.97 (3H, t, 7.42 Hz), 1.60-2.08 (6H, b), 2.38-2.46 (4H, m), 2.73-2.88 (1H, b), 2.90-3.04 (3H, m), 3.10-3.19 (4H, m), 3.48 (3H, bs), 4.05-4.16 (1H, m), 7.27 (2H, d, J = 8.53 Hz), 7.44 (1H, s), 7.66-7.82 (9H, m), 9.04 (2H, d, J = 8.53 Hz) |

### Preparation of N-[4-[(1,1-dioxo-1,4-thiazinan-4-yl)methyl]phenyl]-4-[4-[2-[(3R)-morpholin-3-yl]-1H-imidazol-5-yl]phenyl]benzamide (IIf)

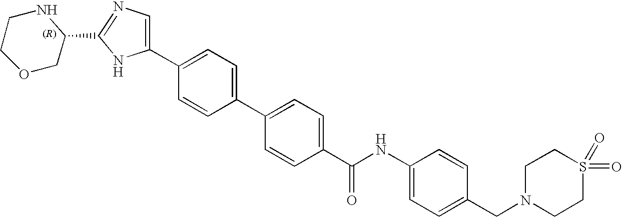

A mixture of tert-butyl (3R)-3-[5-[4-[4-[[4-[(1,1-dioxo-1,4-thiazinan-4-yl)methyl]phenyl]carbamoyl]phenyl]phenyl]-1H-imidazol-2-yl]morpholine-4-carboxylate (14f, 550 mg) and conc hydrochloric acid (5 ml) was stirred in dioxan (10 ml) for 2 h. The mixture was carefully basified with potassium carbonate and then extracted with DCM. The insoluble solid at the liquid interface was then collected by filtration and dried (360 mg)

LC/MS m/z 572

1H NMR (δ, d6 DMSO) 2.79-2.91 (m, 7H), 3.05-3.13 (m, 5H), 3.40-3.56 (m, 3H), 3.63 (s, 2H), 3.62-3.75 (m, 1H), 3.85-3.96 (m, 2H), 7.30 (d, 2H, J=8.84 Hz), 7.54 (s, 1H), 7.76 (2xd, 4H, J=10.74 Hz), 7.85 (2xd, 4H, J=8.21 Hz), 8.05 (d, 2H, J=8.21 Hz)

### Preparation of N-[4-[(1,1-dioxo-1,4-thiazinan-4-yl)methyl]phenyl]-4-[4-[2-[(2S)-2-piperidyl]-1H-imidazol-5-yl]phenyl]benzamide (IIg)

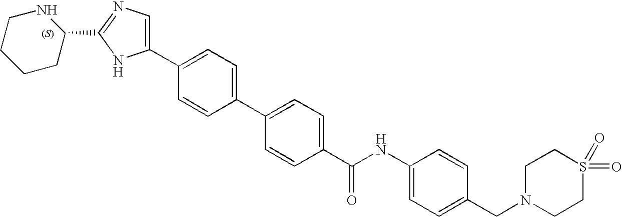

A mixture of tert-butyl (2S)-2-[5-[4-[4-[[4-[(1,1-dioxo-1,4-thiazinan-4-yl)methyl]phenyl]carbamoyl]phenyl]phenyl]-1H-imidazol-2-yl]piperidine-1-carboxylate (14 g, 1.2 g) and conc hydrochloric acid (10 ml) was stirred in dioxan (20 ml) for 18 h. The mixture was carefully basified with potassium carbonate and then extracted with DCM. The insoluble solid at the liquid interface was then collected by filtration and dried (1.01 g). LC/MS no ion seen

TLC (SiO₂) Eluent DCM:EtOH:NH3; 100:8:1 Rf 0.11 (Rf t-butyl ester 0.48)

1H NMR (δ, d6 DMSO) 1.36-1.90 (m, 6H), 2.56-2.68 (m, 1H), 2.83-2.91 (m, 4H), 2.98-3.04 (m, 1H), 3.05-3.18 (m, 4H), 3.62 (s, 2H), 3.67-3.75 (m, 1H), 7.30 (d, 2H, J=8.53 Hz), 7.52 (brs, 1H), 7.71-7.79 (m, 4H), 7.81-7.88 (m, 4H), 8.05 (d, 2H, J=8.21 Hz), 10.31 (s, 1H)

### Preparation of N-[4-[(1,1-dioxo-1,4-thiazinan-4-yl)methyl]phenyl]-4-[2-methyl-4-[2-[(2S)-pyrrolidin-2-yl]-1H-imidazol-5-yl]phenyl]benzamide (IIh)

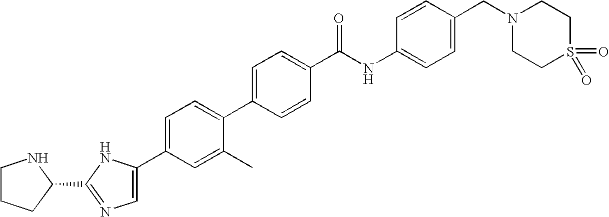

Prepared using the general method of Boc deprotection using 14 h.
Light green solid (98% 0

LC-MS m/z 570, 568

¹H NMR (δ, d₆-DMSO) 1.97-2.45 (8H, b+s), 2.82-3.32 (10H, b), 3.61-3.83 (2H, b), 4.71-4.86 (1H, b), 7.24-7.44 (3H, m), 7.54 (2H, d, J=8.21 Hz), 7.67-7.91 (6H, m), 8.06 (2H, d, J=8.21 Hz), 10.03-10.38 (2H, b+s)

### Alternative procedure for the preparation of N-[4-[(1,1-dioxo-1,4-thiazinan-4-yl)ethyl]phenyl]-4-[4-[2-[(2S)-pyrrolidin-2-yl]-1H-imidazol-5-yl]phenyl]benzamide (IIa)

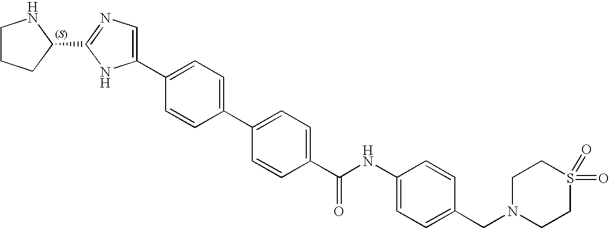

Tert-butyl (2S)-2-[4-[4-[4-[[4-[(1,1-dioxo-1,4-thiazinan-4-yl)methyl]phenyl]carbamoyl]phenyl]phenyl]imidazol-2-yl]pyrrolidine-1-carboxylate (14a, 46.12 g) was suspended in methanol (230 ml) and was heated to 70° C. Conc hydrochloric acid (115 ml) was added and the heat removed. After stirring overnight, the mixture was cooled in an icebath and treated slowly with 10% NaOH solution (500 ml) with stiffing and continued cooling. The resulting solid was removed by filtration, washed with water and dried in vacuo to afford the title compound as yellow solid (38.43 g 98%).

LC-MS m/z 556;

¹H NMR (δ, d₆-DMSO) 1.66-2.15 (4H, b), 2.81-2.90 (4H, m), 3.05-3.15 (4H, m), 3.63 (2H, s), 4.10-4.26 (1H, b), 4.70-4.89 (1H, b), 7.30 (2H, d, J=8.53 Hz), 7.48 (1H, s), 7.73 (2H, d, J=8.21 Hz), 7.77 (2H, d, J=8.21 Hz), 7.84 (4H, d, J=8.53 Hz), 8.04 (2H, d, J=8.21 Hz), 10.32 (1H, s).

### EXAMPLE 1

### Tert-butyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate

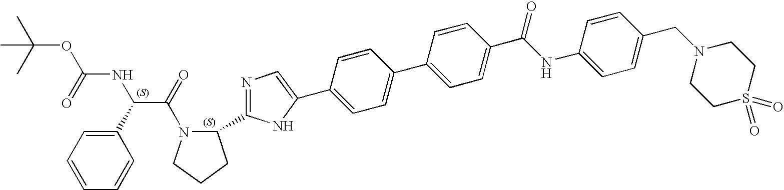

A mixture of N-[4-[(1,1-dioxo-1,4-thiazinan-4-yl)methyl]phenyl]-4-[4-[2-[(2S)-pyrrolidin-2-yl]-1H-imidazol-5-yl]phenyl]benzamide (IIa, 100 mg), HATU (82 mg) and DIPEA (58 mg) in dry DMF (5 ml) was treated with (S)-2-(tert-butoxycarbonylamino)-2-phenylacetic acid (54 mg) and was stirred at 20° C. for 18 h. The mixture was then evaporated and the residue purified by chromatography on silica gel. Elution with 2.5% methanol in DCM gave a colourless solid (50 mg).

LC-MS m/z 789;

¹H NMR (δ, d₆-DMSO) 1.37 (9H, s), 1.85-2.05 (4H, b), 2.80-2.91 (8H, m), 3.04-3.16 (4H, m), 3.63 (2H, s), 5.02-5.16 (1H, m), 5.38-5.48 (1H, m), 7.46-7.26 (8H, m), 7.72-7.92 (8H, m), 8.05 (2H, d, J=8.21 Hz), 10.29 (1H, s).

### EXAMPLE 2

### Tert-butyl N-[(1R)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate

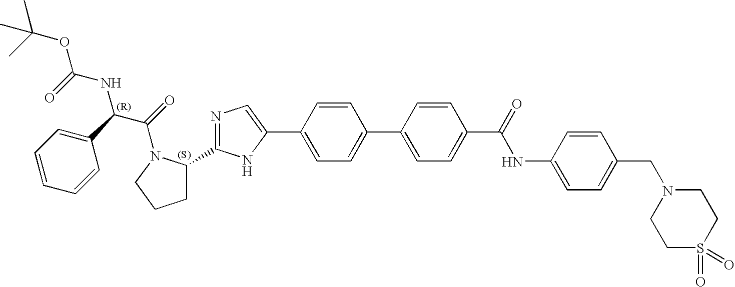

A mixture of N-[4-[(1,1-dioxo-1,4-thiazinan-4-yl)methyl]phenyl]-4-[4-[2-[(2S)-pyrrolidin-2-yl]-1H-imidazol-5-yl]phenyl]benzamide (IIa, 100 mg), HATU (82 mg) and DIPEA (58 mg) in dry DMF (5 ml) was treated with (R)-2-(tert-butoxycarbonylamino)-2-phenylacetic acid (54 mg) and was stirred at 20° C. for 18 h. The mixture was then evaporated and the to residue purified by chromatography on silica gel. Elution with 2.5% methanol in DCM gave a colourless solid (65 mg).

LC-MS m/z 789;

¹H NMR (δ, d₆-DMSO) 1.37 (9H, s), 1.81-2.05 (4H, b), 2.80-2.90 (8H, m), 3.05-3.14 (4H, m), 3.63 (2H, s), 5.01 5.14 (1H, b), 5.38-5.48 (1H, b), 7.27-7.43 (8H, m), 7.71-7.77 is (8H, m), 8.05 (2H, d, J=8.21 Hz), 10.29 (1H, s).

### EXAMPLE 3

### Methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate

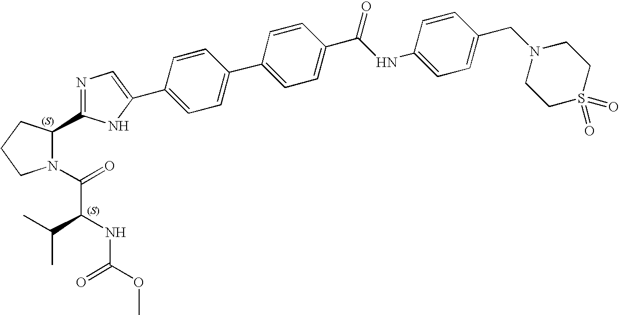

A mixture of N-[4-[(1,1-dioxo-1,4-thiazinan-4-yl)methyl]phenyl]-4-[4-[2-[(2S)-pyrrolidin-2-yl]-1H-imidazol-5-yl]phenyl]benzamide (IIa, 100 mg), HATU (82 mg) and DIPEA (58 mg) in dry DMF (5 ml) was treated with (S)-2-(methoxycarbonylamino)-3-methylbutanoic acid (38 mg) and was stirred at 20° C. for 18 h. The mixture was then evaporated and the residue purified by chromatography on silica gel. Elution with 2.5% methanol in DCM gave an off-white solid (90 mg).

LC-MS m/z 713;

¹H NMR (δ, d₆-DMSO) 0.8-0.91 (6H, m), 1.86-2.23 (4H, b), 2.82-2.91 (8H, m), 3.06-3.13 (4H, m), 3.52 (3H, s), 3.63 (2H, s), 3.76-3.85 (1H, b), 4.0-4.3 (1H, b), 5.05-5.11 (1H, b), 7.31 (2H, d, J=8.51 Hz), 7.71-7.90 (9H, m), 8.04 (2H, d J=8.21 Hz), 10.28 (1H, s).

### EXAMPLE 3(a)

### Larger Scale Preparation of Methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate

Prepared according to Scheme 3.

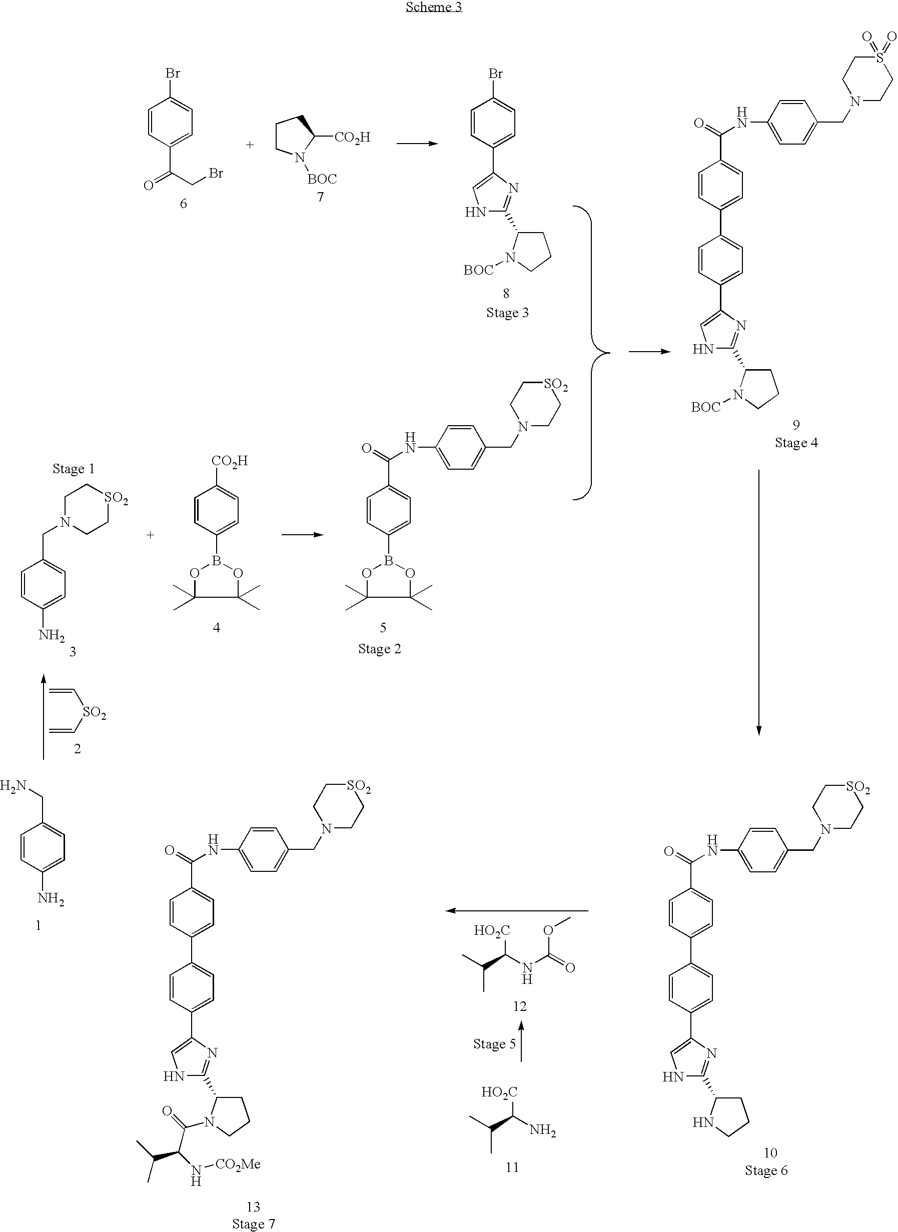

### Stage 1—Preparation of 4-[(1,1-dioxo-1,4-thiazinan-4-yl)methyl]aniline (3)

To a stirred solution of divinylsulfone (2) (606 ml, 6.03 mol) in THF (2680 ml) was added a solution of 4-aminobenzylamine (1) (670 g, 621 ml, 5.48 mol) in THF (670 ml) dropwise under N₂ over 4.5 hours keeping the temperature below 25° C. (using a cold water bath). The mixture was then stirred at room temperature overnight under N₂ after which LC showed the starting material had been consumed. The mixture was warmed to 40° C. and 2 L of solvent removed by vacuum distillation. The mixture was then cooled to less than 15° C. and the solids collected by filtration, washed with THF (800 ml then 400 ml) and pulled dry. The solids were dried overnight in a vacuum oven at 40° C. to give the product (739.6 g, 56%) as a white solid with a purity of 99.1% by LC and >95% by ¹H NMR.

### Stage 2—Preparation of N-[4-[(1,1-dioxo-1,4-thiazinan-4-yl)methyl]phenyl]-4-(4,4,5,5-tetramethyl-1,3,2-dioxaborolan-2-yl)benzamide (5) (also referred to herein as 16a)

To a stirred suspension of 4-[(1,1-dioxo-1,4-thiazinan-4-yl)methyl]aniline (3) (361 g, 1.504 mol) and 4-(4,4,5,5-tetramethyl-1,3,2-dioxaborolan-2-yl)benzoic acid (4) (373 g, 1.504 mol) in MeCN (3.61 L) was added HBTU (570 g, 1.504 mol) and NMM (472 ml, 4.29 mol). The mixture was stirred at room temperature overnight under N₂ after which LC showed 2% of 4-[(1,1-dioxo-1,4-thiazinan-4-yl)methyl]aniline (3) remained. A further charge of 4-(4,4,5,5-tetramethyl-1,3,2-dioxaborolan-2-yl)benzoic acid (4) (7.5 g, 30 mmol) and HBTU (11.4 g, 30 mmol) in MeCN (500 ml) was added and the reaction stirred overnight after which LC showed 1.7% 4-[(1,1-dioxo-1,4-thiazinan-4-yl)methyl]aniline (3) remained. The mixture was filtered, washing the solids with MeCN (2×1 L) and pulled dry. The solids were dried overnight in a vacuum oven at 40° C. ¹H NMR analysis of the crude product (701 g) showed trace amounts of HOBT and NMM were present. The solids were slurried in MeCN (3.5 L) for 2.5 hours then the solids collected by filtration, washed with MeCN (2×μL) and pulled dry. The solids were dried in a vacuum oven at 40° C. over 72 hours to give the product (660 g, 93% yield) as a white solid with a purity of >95% by ¹H NMR with ˜2% residual 4-[(1,1-dioxo-1,4-thiazinan-4-yl)methyl]aniline (3).

For stages 1 and 2 HPLC method was as follows:

Liquid Chromatograph (LC): Agilent 1100 series, with UV detector, scanning at 230 nm.

### LC Conditions:

Mobile phase A: 10 mM ammonium acetate pH 8.0
Mobile phase B: Acetonitrile.

### Gradient:

| Time (min) | % A | % B |
|---|---|---|
| 0 | 100 | 0 |
| 5 | 95 | 5 |
| 15 | 5 | 95 |
| 20 | 5 | 95 |
| 22 | 100 | 0 |

Flow rate: 1.0 ml/min.
Column: XBridge Phenyl 3.5 μM, 4.6 mm×150 mm
Column temperature: 25° C.

### Stage 3—Preparation of tert-butyl (25)-2-[5-(4-bromophenyl)-1H-imidazol-2-yl]pyrrolidine-1-carboxylate (8)

To a 5 L flask was charged N-Boc-L-proline (7) (335 g, 1.558 mol, 1.05 Eq) followed by 2,4′-dibromoacetophenone (6) (412.4 g, 1.4838 mol, 1 Eq), xylenes (3.0 L) and MeCN (335 mL). The reaction mixture was stirred for 5 minutes and DIPEA (269.6 mL, 1.558 mol, 1.05 Eq) was added. The reaction was then warmed to 30-33° C. and stirred at this temperature for 22 hours after which <1% of the dibromoacetophenone remained by LC. The separated solid was filtered and the filtrate was charged to a 10 L flask followed by NH₄OAc (1.14 Kg, 14.838 mol, 10 Eq). The suspension was heated to 110° C. while distilling off the MeCN. After 335 mL of solvent had distilled the reaction was set-up for reflux. The reaction was refluxed for 5 hours after which TLC/LC-MS indicated that all the starting material (phenacyl ester of proline) had been consumed. The reaction was cooled to room temperature and the lower layer of the reaction mixture was separated and the upper xylenes layer was washed with saturated NaHCO₃ (2×1 L). The lower layer of the reaction separated was back extracted with EtOAc (1×1 L) and this EtOAc was washed with saturated NaHCO₃ (2×300 mL) and then combined with the rest of the organic/xylenes layer. The combined organic layers were dried (MgSO₄) and during this process the product started to precipitate out. The suspension was filtered and MgSO₄ washed with EtOAc (3 L). Evaporation of the organics under vacuum afforded a yellow solid which was slurried in heptane (1 L) and MTBE (500 mL) for 15 minutes, filtered and washed with 1:1 heptane-MTBE (1 L). The product was pulled dry for 30 minutes to give 510 g of crude product as an off white solid. The ¹H-NMR showed that the crude product contained about 3.5% of heptane and by LC the purity was 99.4%. The crude product was then dried under vacuum at 40° C. to give 470 g of the product (77% yield) with a purity of >99% by LC and >95% by ¹H NMR. The enantiomeric excess was measured to be >99%.

For stage 3, the HPLC method for enantiomeric excess determination was as follows:

Liquid Chromatograph (LC): Agilent 1100 series, with UV detector, scanning at 274 nm.

### LC Conditions:

Mobile phase: Hexane:ethanol (70:30)
Flow rate: 1.0 ml/min.
Column: Chiralpak 1A 250 mm×4.6 mm, 51 am particle size
Column temperature: 40° C.

### Stage 4—Preparation of tert-butyl (2S)-2-[4-[4-[4-[[4-[(1,1-dioxo-1,4-thiazinan-4-yl)methyl]phenyl]carbamoyl]phenyl]phenyl]imidazol-2-yl]pyrrolidine-1-carboxylate (9) (also referred to herein as 14a)

A stirred suspension of tert-butyl (2S)-2-[5-(4-bromophenyl)-1H-imidazol-2-yl]pyrrolidine-1-carboxylate (8) (411.5 g, 1.05 mol), N-[4-[(1,1-dioxo-1,4-thiazinan-4-yl)methyl]phenyl]-4-(4,4,5,5-tetramethyl-1,3,2-dioxaborolan-2-yl)benzamide (5) (493.5 g, 1.05 mol) and Cs₂CO₃ (1163 g, 3.57 mol) in DME (5330 ml) and water (1925 ml) was vacuum degassed with N₂ five times. The suspension was heated to 80° C. then Pd(PPh₃)₄ (12.1 g, 10.5 mmol) added. LC analysis after 4 hours indicated 0.5% boronate remained. The reaction was left to cool to room temperature overnight after which a beige precipitate had formed. The solids were collected by filtration and pulled dry. The solids were slurried in water (7 L) for 30 minutes then filtered overnight (slow filtration due to fine particles). The solids were then slurried in acetone (5 L) for 2 hours then filtered, washing with acetone (2 L) and pulled dry. Solids were dried overnight in a vacuum oven at 45° C. to give the product (503.8 g, 73% yield) as a white solid with a purity of 97.5% by LC and >95% by ¹H NMR.

### Stage 5—Preparation of (S)-2-(methoxycarbonylamino)-3-methylbutanoic acid (12) (also referred to herein as A3)

A stirred solution of NaOH (97.7 g, 2.44 mol) in water (650 ml) was cooled to less than 15° C. L-Valine (130 g, 1.11 mol) was added in one portion and the mixture stirred until all solids were dissolved. The solution was then cooled to 0° C. and a solution of methyl chloroformate (94 ml, 1.22 mol) in toluene (650 ml) added slowly keeping the temperature below 5° C. (ice/acetone bath used, addition time about 1 hour). After 2 hours, TLC analysis (EtOAc eluent, product Rf ˜0.5 with ninhydrin stain) showed the starting material was consumed. The aqueous layer was separated and cooled to less than 10° C. A solution of 5M H₂SO_{4(aq)} (260 ml, 1.30 mol) was added in portions with stirring keeping the temperature below 25° C. After about ⅓ of the acid solution was added (foaming/gas evolution was observed), a large quantity of precipitate started to form and foam up. This was prevented by addition of EtOAc (330 ml) to dissolve the precipitate and then continuing with the acidification. After the addition of acid was complete, the layers were separated. The aqueous layer was extracted with EtOAc (2×330 ml). The EtOAc layers were combined and washed with water (300 ml). The organics were dried (MgSO₄) and concentrated under reduced pressure to give the product (171.4 g, 88% yield) as a white solid with a purity of >95% by ¹H NMR and 98% by GC.

For stage 5, the gas chromatography method was as follows:

### GC Conditions:

### Carrier Gas:Nitrogen

Head Pressure: 12 psi, constant pressure
Column: DB-1, 30m×0.32 mm, 1.0 μm film thickness
Oven Program: 40° C. (Hold 5 mins) then 20° Cmin⁻¹ to 300° C. (Hold 10 mins)
Injector Temperature: 200° C., split
Column Temperature: 250° C., Flame ionization detector
Liner:SGE Focusliner with glass wool insert

### Stage 6—Preparation of N-[4-[(1,1-dioxo-1,4-thiazinan-4-yl)methyl]phenyl]-4-[4-[2-[(2S)-pyrrolidin-2-yl]-1H-imidazol-5-yl]phenyl]benzamide (10) (also referred to herein as IIa)

Tert-butyl(2S)-2-[4-[4-[4-[[4-[(1,1-dioxo-1,4-thiazinan-4-yl)methyl]phenyl]carbamoyl]phenyl]phenyl]imidazol-2-yl]pyrrolidine-1-carboxylate (9) (46 g, 0.07 mol) was suspended in methanol (200 ml) at 30° C. and c.HCl (100 ml) was added. The reaction was stirred at 30° C. for 3 hours, then chilled in an ice-bath and 10% aqueous NaOH solution (500 ml) added. The resulting solid was filtered, washed with water, slurried with acetonitrile and then filtered and pulled dry. Solids were dried overnight in a vacuum oven at 45° C. to give the product (37.2 g, 95% yield) with a purity of 97.3% by LC.

### Stage 7—Preparation of Methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate (13)

N-[4-[(1,1-dioxo-1,4-thiazinan-4-yl)methyl]phenyl]-4-[4-[2-[(2S)-pyrrolidin-2-yl]-1H-imidazol-5-yl]phenyl]benzamide (10) (46.7 g, 0.084 mol), (S)-2-(methoxycarbonylamino)-3-methylbutanoic acid (12) (16.24 g, 0.092 mol) and HBTU (48.1 g, 0.126 mol) were stirred in DMF (275 ml) and NMM (20.4 ml, 0.185 mol) at ambient temperature for 4 hours, then poured slowly onto stirred water (1500 ml) to produce a pale cream solid. This solid was filtered, washed with water and dried in vacuo, before batchwise purification on a pad of silica, using 100% ethyl acetate, 5% ethanol/ethylacetate, 10% ethanol/ethylacetate, 20% ethanol/ethylacetate gradient. The fractions containing product were concentrated in vacuo and the resulting solid was dissolved in acetone (50 ml) and then poured slowly into diethyl ether (1000 ml). The solid that precipitated was removed by filtration, washed with diethyl ether and dried in vacuo at 40° C. to give the product (54 g, 90%).

¹H NMR (δ, d₆-DMSO) 0.8-0.91 (6H, m), 1.86-2.23 (4H, b), 2.82-2.91 (8H, m), 3.06-3.13 (4H, m), 3.52 (3H, s), 3.63 (2H, s), 3.76-3.85 (1H, b), 4.0-4.3 (1H, b), 5.05-5.11 (1H, b), 7.31 (2H, d, J=8.51 Hz), 7.71-7.90 (9H, m), 8.04 (2H, d J=8.21 Hz), 10.28 (1H, s).

For stages 4, 6 and 7, the LC method/conditions were as follows:

Liquid Chromatograph (LC): Agilent 1100 series, with UV detector, scanning at 230 nm.

### LC Conditions:

Mobile phase A: Purified water: TFA (100:0.1)
Mobile phase B: Acetonitrile: TFA (100:0.1)

### Gradient:

| Time (min) | % A | % B |
|---|---|---|
| 0 | 95 | 5 |
| 15 | 5 | 95 |
| 20 | 5 | 95 |
| 22 | 95 | 5 |

Flow rate: 1.0 ml/min.
Column: XBridge Phenyl 3.5 μM, 4.6 mm×150 mm
Column temperature: 30° C.

### EXAMPLE 4

### Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxoethyl]carbamate

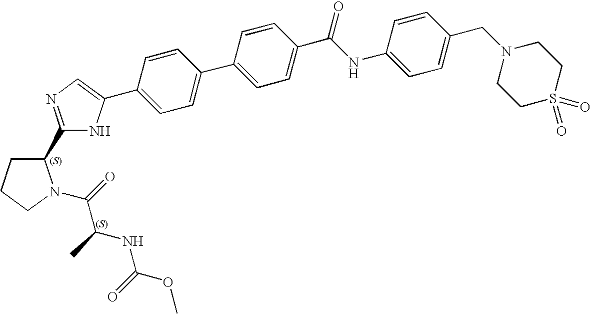

A mixture of (2S)-2-(methoxycarbonylamino)propanoic acid (A4, 0.18 mmol, 1 eq), HBTU (0.19 mmol, 1.05 eq), dry DMF (4 ml), and NMM (0.72 mmol, 4 eq), was stirred under nitrogen for 0.5 h at room temperature at which stage N-[4-[(1,1-dioxo-1,4-thiazinan-4-yl)methyl]phenyl]-4-[4-[2-[(2S)-pyrrolidin-2-yl]-1H-imidazol-5-yl]phenyl]benzamide (IIa, 0.18 mmol, 1 eq) was added and the mixture left to stir overnight. The mixture was then concentrated to dryness and put on a SPE column and eluted with 2.5-5% MeOH:DCM to yield the pure compound as an off white solid (16%)

LC-MS m/z 685;

¹H NMR (δ, (δ, d₆-DMSO) 1.20 (3H, d, J=6.95 Hz), 1.84-2.20 (4H, b), 2.82-2.90 (4H, m), 3.05-3.15 (4H, m), 3.31 (3H, s), 3.63-3.81 (4H, s+b), 4.23-4.41 (1H, m), 5.03-5.16 (1H, m), 7.26-7.29 (3H, m), 7.55 (1H, s), 7.69-7.94 (8H, m), 8.04 (2H, d, J=8.53 Hz), 10.28 (1H, s), 11.76 (0.5H, s), 12.06-12.09 (0.5H, b).

The following examples were prepared by the method of Example 4 using N-[4-[(1,1-dioxo-1,4-thiazinan-4-yl)methyl]phenyl]-4-[4-[2-[(2S)-pyrrolidin-2-yl]-1H-imidazol-5-yl]phenyl]benzamide (IIa) and the appropriate carboxylic acid.

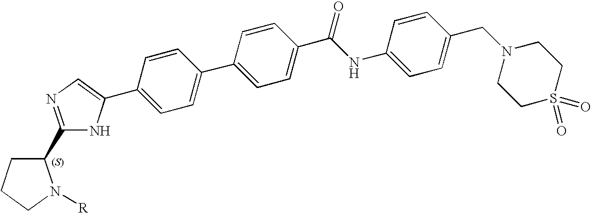

| | | | LC- | |
|---|---|---|---|---|
| R group | Name | Yield | MS | 1H NMR |
| Example 5, | Methyl N-[(1S)-1- | White | m/z | 0.93 (9H, s), 1.86-2.25 |
| R = | [(2S)-2-[5-[4-[4-[[4- | solid | 727 | (4H, m), 2.79-2.95 (4H, |
| 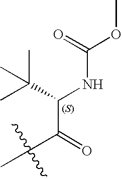 | [(1,1-dioxo-1,4- thiazinan-4- yl)methyl]phenyl] carbarmoyl]phenyl]phenyl]- 1H-imidazol-2- yl]pyrrolidine-1- carbonyl]-2,2- dimethyl- propyl]carbamate | (38 mg, 29%) | | m), 3.04-3.20 (4H + 2H, m), 3.54 (3H, s), 3.63 (2H, s), 4.07-4.25 (1H, m), 5.09 (1H, br s), 7.30 (2H, d, J = 8.5 Hz), 7.49- 7.58 (1H, m), 6.69-7.93 (8H, m), 7.94-8.10 (2H + 1H, d + m, J = 7.6 Hz), 10.28 (1H, s), 11.85 (0.5H, br s) |
| Example 6, | Methyl N-[(1S)-2- | Yellow | m/z | 1.81-2.07 (4H, b), 2.81- |
| R = | [(2S)-2-[5-[4-[4-[[4- | solid | 747 | 2.90 (6H, m), 3.06-3.15 |
| 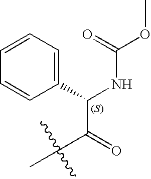 | [(1,1-dioxo-1,4- thiazinan-4- yl)methyl]phenyl] carbamoyl]phenyl]phenyl]- 1H-imidazol-2- yl]pyrrolidin-1-yl]-2- oxo-1-phenyl- ethyl]carbamate | (29%) | | (4H, m), 3.51-3.56 (3H, m), 3.64 (2H, s), 5.02- 5.17 (1H, m), 5.44-5.56 (1H, m), 7.27-7.91 (15H, m), 7.94 (1H, s), 8.06 (2H, d , J = 8.53 Hz), 10.28 (1H, s), 11.78 and 11.98 (1H, 2 x s) |
| Example 7, | Methyl N-[(1R)-2- | Yellow | m/z | 1.80-2.07 (4H, b), 2.79- |
| R = | [(2S)-2-[5-[4-[4-[[4- | oil | 747 | 2.93 (6H, m), 3.03-3.15 |
| 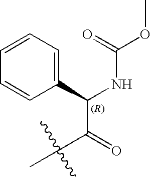 | [(1,1-dioxo-1,4- thiazinan-4- yl)methyl]phenyl] carbamoyl]phenyl]phenyl]- 1H-imidazol-2- yl]pyrrolidin-1-yl]-2- oxo-1-phenyl- ethyl]carbamate | (29%) | | (4H, m), 3.46-3.56 (3H, m), 3.64 (2H, s), 5.02- 5.17 (1H, m), 5.32-5.54 (1H, m), 6.90-7.92 (16H, m), 7.94 (1H, s), 8.05 (2H, d, J = 8.53 Hz), 10.28 (1H, s), 11.74- 12.00 (1H, b) |
| Example 8, | 4-[4-[2-[(2S)-1-[(2R)- | Oil | m/z | 0.99-1.20 (6H, m), 1.80- |
| R = | 2-(diethylamino)-2- | (30%) | 745 | 2.15 (4H, b), 2.45-2.51 |
| 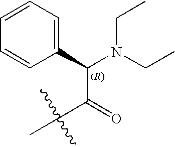 | phenyl- acetyl]pyrrolidin-2-yl]- 1H-imidazol-5- yl]phenyl]-N-[4-[(1,1- dioxo-1,4-thiazinan-4- yl)methyl]phenyl] benzamide | | | (4H, m), 2.82-2.92 (4H, m), 3.06-3.16 (4H, m), 3.30-3.40 (2H, m), 3.64 (2H, s), 5.02-5.09, (1H, m), 5.34-5.42 (1H, m), 7.27-8.08 (19H, m), 10.28 (1H, s) |
| Example 9, | 4-[4-[2-[(2S)-1-[(2R)- | Oil | m/z | 1.8-2.12 (4H, b), 2.2.53- |
| R = | 2-(dimethylamino)-2- | (24%) | 717 | 2.65 (6H, m), 3.05-3.16 |
| 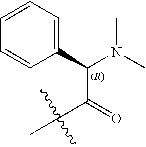 | phenyl- acetyl]pyrrolidin-2-yl]- 1H-imidazol-5- yl]phenyl]-N-[4-[(1,1- dioxo-1,4-thiazinan-4- yl)methyl]phenyl] benzamide | | | (4H, m), 3.3-3.41 (2H, b), 3.64 (2H, s), 5.01- 5.09 91H, m), 5.21-5.30, 5.34-5.50 (1H, 2 x m), 7.31 (2H, d, J = 8.50 Hz), 7.37-7.90 (14H, m), 7.97 (1H, d, J = 8.53 Hz), 8.05 (2H, d, J = 8.53 Hz), 10.28 (1H, s) |
| Example 10, 4-[4-[2- [(2S)-1- [(2S)-2- amino-3- methyl- butanoyl] pyrrolidin-2-yl]- 1H-imidazol- 5-yl]phenyl]- N-[4-[(1,1- dioxo-1,4- thiazinan-4- yl)methyl]phenyl] benzamide | 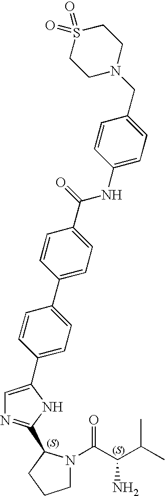 | White solid (777 mg, 86%) | m/z 655 | 0.76-0.90 (6H, m), 1.68- 2.27 (5H, m), 2.87 (4H, m), 3.03-3.16 (4H + 1H, m), 3.25-3.47 (2H, m), 3.55-3.72 (2H + 2H, s + m), 5.05-5.20 (1H, 2 x m), 7.30 (2H, d, J = 8.8 Hz), 7.54-7.87 (9H, m), 8.04 (2H, d, J = 7.6 Hz), 10.28 (1H, s), 11.75 (0.5H, s) |
| Example 11, | Methyl N-[(1S)-2- | Clear | m/z | 1.83-2.27 (5H, m), 2.87 |
| R = | [(2S)-2-[5-[4-[4-[[4- | foam | 701 | (4H, m), 3.10 (4H, m), |
| 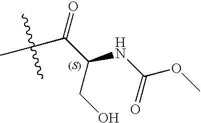 | [(1,1-dioxo-1,4- thiazinan-4- yl)methyl]phenyl] carbamoyl]phenyl]phenyl]- 1H-imidazol-2- yl]pyrrolidin-1-yl]-1- (hydroxymethyl)-2- oxo-ethyl]carbamate | (19 mg, 30%) | | 3.32-3.49 (3H, m), 3.53 (2H, s), 3.59-3.70 (3H + 1H, s + m), 4.95-5.15 (1H, m), 7.30 (3H, d, J = 8.2 Hz), 7.56-7.89 (10H, m), 8.04 (2H, d, J = 8.2 Hz), 10.28 (1H, s), 11.77-12.24 (1H, m) |
| Example 12, | tert-Butyl N-[2-[(2S)-2- | Off | m/z | 1.33, 1.36 (9H, 2 x s), |
| R = | [5-[4-[4-[[4-[(1,1- | white | 713 | 1.84-2.18 (4H, m), 2.86 |
| 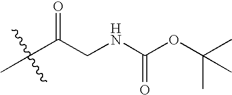 | dioxo-1,4-thiazinan-4- yl)methyl]phenyl] carbamoyl]phenyl]phenyl]- 1H-imidazol-2- yl]pyrrolidin-1-yl]-2- oxo-ethyl]carbamate | solild (50 mg, 55%) | | (4H, m), 3.10 (4H, m), 3.43-3.53 (1H, m), 3.58- 3.69 (2H + 1H, s + m), 3.70-3.84 (2H, m), 5.04- 5.18 (1H, m), 6.68-6.77 (1H, m), 7.30 (2H, d, J = 8.5 Hz), 7.55-7.90 (9H, m), 8.04 (2H, d, J = 8.2 Hz), 10.28 (1H, s), 11.80, 12.19 (1H, 2 x s) |
| Example 13, | Methyl N-[(1R)-1- | Tan | m/z | 0.26, 0.68, 0.88 (6H, 3 x |
| R = | [(2S)-2-[5-[4-[4-[[4- | foam | 713 | d, J = 6.5 Hz), 1.57-2.29 |
| 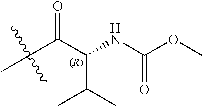 | [(1,1-dioxo-1,4- thiazinan-4- yl)methyl]phenyl] carbamoyl]phenyl]phenyl]- 1H-imidazol-2- yl]pyrrolidine-1- carbonyl]-2-methyl- propyl]carbamate | (34 mg, 50%) | | (5H, m), 2.78-2.89 (5H, m), 3.10 (4H, m), 3.48- 3.58 (4H, m), 3.64 (2H, s), 3.82-4.15 (1H, m), 5.62, 5.08 (1H, 2 x m), 7.28-7.43 (3H, m), 7.63- 7.94 (9H, m), 8.02-8.07 (2H, m), 10.28 (1H, s) |
| Example 14, | Methyl N-[(1S)-1- | Tan | m/z | 0.81-0.99 (6H, m), 1.41- |
| R = | [(2S)-2-[5-[4-[4-[[4- | foam | 727 | 1.79 (3H, m), 1.99-2.28 |
| 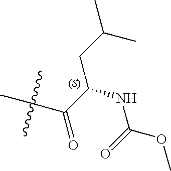 | [(1,1-dioxo-1,4- thiazinan-4- yl)methyl]phenyl] carbamoyl]phenyl]phenyl]- 1H-imidazol-2- yl]pyrrolidine-1- carbonyl]-3-methyl- butyl]carbamate | (30 mg, 43%) | | (4H, m), 2.94 (4H, m), 3.16 (4H, m), 3.43-3.53 (1H, m), 3.59 (3H, s), 3.71 (2H, s), 3.79 (1H, m), 4.05-4.43 (1H, 2 x m), 5.15 (1H, m), 7.36- 7.43 (2H + 1H, d + m, J = 8.5 Hz), 7.63-8.01 (9H, m), 8.11 (2H, d, J = 8.5 Hz), 10.35 (1H, s), 11.85 (0.5H, s) |
| Example 15, | Methyl N-[(1R)-1- | Yellow | m/z | 0.26-0.92 (6H, m), 1.32- |
| R = | [(2S)-2-[5-[4-[4-[[4- | foam | 727 | 1.68 (3H, m), 1.82-1.98 |
| 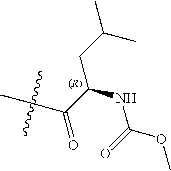 | [(1,1-dioxo-1,4- thiazinan-4- yl)methyl]phenyl] carbamoyl]phenyl]phenyl]- 1H-imidazol-2- yl]pyrrolidine-1- carbonyl]-3-methyl- butyl]carbamate | (30 mg, 43%) | | (4H, m), 2.05-2.32 (1H, m), 2.86 (4H, m), 3.09 (4H, m), 3.35-3.46 (1H, m), 3.52 (3H, s), 3.63 (2H, s), 3.87-3.79 (1H, m), 4.08-4.38 (1H, 2 x m), 5.04, 5.49 (1H, 2 x m), 7.30 (2H, d, J = 8.5 Hz), 7.51, 7.60 (1H, 2 x s), 7.72-7.91 (9H, m), 8.04 (2H, d, J = 8.2 Hz) |
| Example 16, | Methyl N-[(1S,2S)-1- | Yellow | m/z | 0.82-0.94 (6H, m), 1.20 |
| R = | [(2S)-2-[5-[4-[4-[[4- | glass | 727 | (1H, m), 1.53 (1H, m), |
| 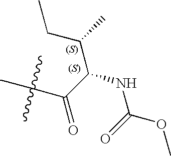 | [(1,1-dioxo-1,4- thiazinan-4- yl)methyl]phenyl] carbamoyl]phenyl]phenyl]- 1H-imidazol-2- yl]pyrrolidine-1- carbonyl]-2-methyl- butyl]carbamate | (8 mg, 11%) | | 1.78 (1H, m), 1.98-2.29 (4H, m), 2.94 (4H, m), 3.18 (4H, m), 3.60 (3H, s), 3.71 (2H, s), 3.89 (2H, m), 4.18 (1H, t, J = 8.8 Hz), 5.16 (1H, m), 7.38 (2H, d, J = 8.8 Hz), 7.62 (1H, s), 7.78-8.01 (9H, m), 8.11 (2H, d, J = 8.2 Hz), 10.35 (1H, s), 11.90 (1H, s) |
| Example 17, | Methyl N-[(1S,2R)-1- | Yellow | m/z | 1.03-1.14 (3H, m), 1.88- |
| R = | [(2S)-2-[5-[4-[4-[[4- | glass | 715 | 2.23 (4H, m), 2.86 (4H, |
| 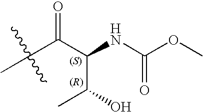 | [(1,1-dioxo-1,4- thiazinan-4- yl)methyl]phenyl] carbamoyl]phenyl]phenyl]- 1H-imidazol-2- yl]pyrrolidine-1- carbonyl]-2-hydroxy- propyl]carbamate | (7 mg, 10%) | | m), 3.09 (4H, m), 3.54 (3H, s), 3.63 (2H, s), 3.75-3.93 (3H, m), 4.29 (1H, d, J = 6.3 Hz), 5.12 (1H, m), 7.30 (2H, d, J = 8.5 Hz), 7.51 (1H, s), 7.71-7.87 (9H, m), 8.04 (2H, d, J = 8.5 Hz) |
| Example 18, | Methyl N-[(1R,2S)-1- | Yellow | m/z | 0.62, 1.08 (3H, 2 x d, J = |
| R = | [(2S)-2-[5-[4-[4-[[4- | glass | 715 | 6.3 Hz), 1.95-2.20 (4H, |
| 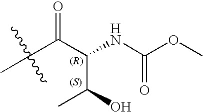 | [(1,1-dioxo-1,4- thiazinan-4- yl)methyl]phenyl] carbamoyl]phenyl]phenyl]- 1H-imidazol-2- yl]pyrrolidine-1- carbonyl]-2-hydroxy- propyl]carbamate | (7 mg, 10%) | | m), 2.87 (4H, m), 3.10 (4H, m), 3.56 (3H., s), 3.63 (2H, s), 3.72-3.93 (2H, m), 4.10, 4.28 (1H, 2 x d, J = 6.0 Hz), 5.06 (1H, m), 5.55 (1H, m), 7.30 (2H, d, J = 8.5 Hz), 7.51, 7.59 (1H, 2 x s), 7.71-7.87 (9H, m), 8.04 (2H, d, J = 8.2 Hz) |
| Example 19, | Methyl N-[(1R)-2- | Yellow | m/z | 1.79-2.30 (5H, m), 2.87 |
| R = | [(2S)-2-[5-[4-[4-[[4- | glass | 701 | (4H, m), 3.09 (4H, m), |
| 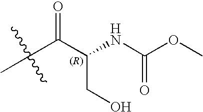 | [(1,1-dioxo-1,4- thiazinan-4- yl)methyl]phenyl] carbamoyl]phenyl]phenyl]- 1H-imidazol-2- yl]pyrrolidin-1-yl]-1- (hydroxymethyl)-2- oxo-ethyl]carbamate | (7 mg, 10%) | | 3.45-3.61 (5H, m), 3.63 (2H, s), 3.67-3.86 (1H, m), 4.25, 4.42 (1H, 2 x m), 5.07 (1H, m), 7.30 (2H, d, J = 8.5 Hz), 7.51- 7.62 (1H, m), 7.72-7.92 (8H, m), 8.04 (2H, d, J = 8.5 Hz) |

NB: Example 10 was prepared by the general method of Example 4 using A10 followed by isolation of the crude material and deprotection according to the general method of Boc deprotection

The following examples were prepared by the method of Example 4 using N-[4-[(1,1-dioxo-1,4-thiazinan-4-yl)methyl]phenyl]-4-[4-[2-[(2R)-pyrrolidin-2-yl]-1H-imidazol-5-yl]phenyl]benzamide (IIb) and the appropriate carboxylic acid.

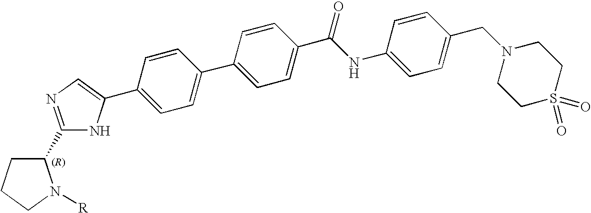

| | | | LC- | |
|---|---|---|---|---|
| R group | Name | Yield | MS | 1H NMR |
| Example 20, | Methyl N-[(1S)-1- | Pale | m/z | 0.27 and 0.69 (3H, d, J = |
| R = | [(2R)-2-[5-[4-[4-[[4- | yellow | 713 | 6.63 Hz), 0.91 (3H, d, |
| 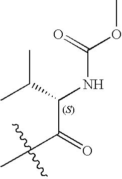 | [(1,1-dioxo-1,4- thiazinan-4- ylmethyl]phenyl] carbamoyl]phenyl]phenyl]- 1H-imidazol-2- yl]pyrrolidine-1- carbonyl]-2-methyl- propyl]carbamate | solid (41%) | | 6.63 Hz), 1.80-2.25 (4H, b), 2.83-2.91 (4H, m), 3.07-3.17 (4H, m), 3.48- 3.68 (7H, 2 x s + b), 3.99- 4.15 (2H, m), 5.05-5.12 and 5.59-5.66 (1H, 2 x b), 7.03-7.74 (14H, m), 10.29 (1H, s), 11.50- 11.55 and 12,10-12.16 (1H, 2 x b) |
| Example 21, | Methyl N-[(1S)-2- | Off | m/z | 0.84 and 1.28 (2H, d, J = |
| R = | [(2R)-2-[5-[4-[4-[[4- | white | 685 | 6.54 Hz), 1.81-2.40 (4H, |
| 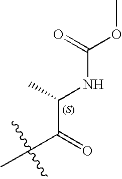 | (1,1-dioxo-1,4- thiazinan-4- yl)methyl]phenyl] carbamoyl]phenyl] phenyl]-1H-imidazol- 2-yl]pyrrolidin-1-yl]- 1-methyl-2-oxo- ethyl]carbamate | solid (26%) | | b), 2.89-2.98 (4H, m), 3.14-3.22 (4H, m), 3.58- 3.72 (7H, m), 4.23-4.29 (1H, m), 5.10-5.12 and 5.44-5.52 (1H, 2 x b), 7.34-8.21 (14H, m), 10.36 (1H, s), 11.52- 11.60 and 12.26-12.34 (1H, 2 x b) |
| Example 22, | Methyl N-[(1S)-1- | White | m/z | 0.68 (3H, s), 1.04 (6H, s), |
| R = | [(2R)-2-[5-[4-[4-[[4- | solid | 727 | 1.88-2.40 (4H, b), 2.81- |
| 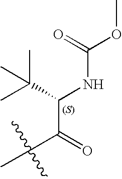 | [(1,1-dioxo-1,4- thiazinan-4- yl)methyl]phenyl] carbamoyl]phenyl]phenyl]- 1H-imidazol-2- yl]pyrrolidine-1- carbonyl]-2,2- dimethyl- propyl]carbamate | (19%) | | 3.00 (4H, m), 3.14-3.24 (4H, m), 3.53-3.79 (7H, 2 x s + b), 3.84-3.99 (1H, b), 4.28-4.36 (1H, d, J = 8.85 Hz), 5.11-5.19 (1H, b), 7.21-8.14 (14H, m), 10.36 (1H, s), 11.64 and 12.13 (1H, 2 x s) |
| Example 23, | Methyl N-[(1S)-2- | Yellow | m/z | 1.81-2.21 (4H, b), 2.89- |
| R = | [(2R)-2-[5-[4-[4-[[4- | solid | 747 | 2.98 (4H, m), 3.12-3.23 |
| 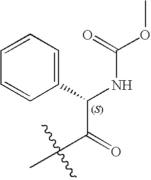 | [(1,1-dioxo-1,4- thiazinan-4- yl)methyl]phenyl] carbamoyl]phenyl]phenyl]- 1H-imidazol-2- yl]pyrrolidin-1-yl]-2- oxo-1-phenyl- ethyl]carbamate | (40%) | | (5H, m), 3.57-3.60 (3H, m), 3.71 (2H, s), 3.74- 4.02 (1H, b), (5.09-5.24 (1H, m), 5.47-5.62 (1H, m), 7.13-8.16 (19H, m), 10.35 (1H, s), 11.81- 12.12 (1H, b) |
| Example 24, | Methyl N-[(1R)-2- | Yellow | m/z | 1.82-2.21 (4H, b), 2.81- |
| R = | [(2R)-2-[5-[4-[4-[[4- | solid | 747 | 2.92 (4H, m), 3.06-3.17 |
| 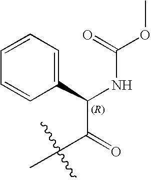 | [(1,1-dioxo-1,4- thiazinan-4- yl)methyl]phenyl] carbamoyl]phenyl] phenyl]-1H-imidazol- 2-yl]pyrrolidin-1-yl]- 2-oxo-1-phenyl- ethyl]carbamate | (42%) | | (5H, m), 3.50-3.57 (3H, m)), 3.64 (2H, s), 3.74- 4.02 (1H, b), 5.03-5.19 (1H, m), 5.44-5.55 (1H, m), 7.27-8.08 (19H, m), 10.28 (1H, s), 11.73- 12.04 (1H, b) |
| Example 25, | 4-[4-[2-[(2R)-1- | White | m/z | 0.57 (3H, t, J = 7.17 Hz), |
| R = | [(2R)-2- | solid | 745 | 0.90 (3H, t, J = 7.17 Hz), |
| 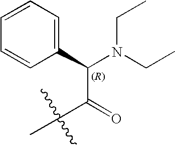 | (diethylamino)-2- phenyl- acetyl]pyrrolidin-2- yl]-1H-imidazol-5- yl]phenyl]-N-[4- [(1,1-dioxo-1,4- thiazinan-4- yl)methyl]phenyl] benzamide | (23%) | | 1.84-2.20 (4H, m), 2.24- 2.75 (4H, m), 2.82-2.91 (4H, m), 3.06-3.15 (4H, m), 3.36-3.51 (1H, b), 3.64 (2H, s), 3.64-3.80 (1H, b), 4.43-4.48 (0.5H, b), 4.65-4.81 (1H, m), 5.09-5.16 (0.5H, m), 7.23-7.07 (19H, m), 10.29 (1H, s), 11.73- 12.48 (1H, b) |
| Example 26, | 4-[4-[2-[(2R)-1- | White | m/z | 1.80-2.21 (4H, b), 2.35 |
| R = | [(2R)-2- | solid | 717 | (3H, s), 2.88-2.98 (4H, |
| 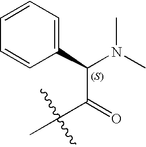 | (dimethylamino)-2- phenyl- acetyl]pyrrolidin-2- yl]-1H-imidazol-5- yl]phenyl]-N-[4- [(1,1-dioxo-1,4- thiazinan-4- yl)methyl]phenyl] benzamide | (51%) | | m), 3.13-3.22 (4H, m), 3.68 (2H, s), 3.64-3.86 (2H, b), 3.98 (0.6H, s), 4.63 (0.4H, s), 4.99-5.06 (0.5H, m), 5.17-5.24 (0.5H m), 7.34-7.62 (8H, m), 7.76-8.0 (10H, m), 8.12 (2H, d, J = 8.21 Hz), 10.36 (1H, s) |
| Example 27, | methyl N-[(1R)-1- | White | m/z | ¹H NMR (δ, d₆-DMSO) |
| R = | [(2R)-2-[5-[4-[4-[[4- | solid | 713, | 0.79-0.96 (6H, m), 1.80- |
| 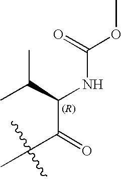 | [(1,1-dioxo-1,4- thiazinan-4- yl)methyl]phenyl] carbamoyl]phenyl] phenyl]-1H-imidazol-2- yl]pyrrolidine-1- carbonyl]-2-methyl- propyl]carbamate | (16%) | 711 | 2.30 (5H, b), 2.80-2.93 (4H, b), 3.05-3.20 (4H, b), 3.53 (3H, s), 3.60- 3.68 (2H, b), 3.75-3.88 (2H, b), 3.95-4.15 (1H, b), 5.03-5.14 (0.5H, b), 7.20-8.14 (15H, m), 9.99 (0.2H, s), 10.06-10.14 (0.2H, b), 10.29 (1H, s), 11.81-11.88 (0.5H, b) |

The following examples were prepared by the method of Example 4 using N-[4-[(1,1-dioxo-1,4-thiazinan-4-yl)methyl]phenyl]-3-[4-[2-[(2S)-pyrrolidin-2-yl]-1H-imidazol-5-yl]phenyl]benzamide (IIc) and the appropriate carboxylic acid.

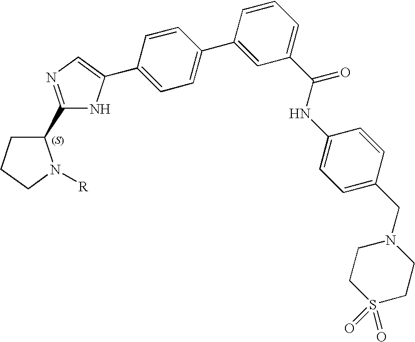

| | | | LC- | |
|---|---|---|---|---|
| R group | Name | Yield | MS | 1H NMR |
| Example 28, | Methyl N-[(1S)-1- | Yellow | m/z | 0.81-0.93 (6H, m), 1.87- |
| R = | [(2S)-2-[5-[4-[3-[[4- | solid | 713 | 2.23 (4H, b), 2.81-2.91 |
| 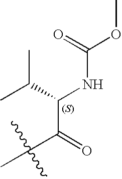 | [(1,1-dioxo-1,4- thiazinan-4- yl)methyl]phenyl] carbamoyl]phenyl] phenyl]-1H-imidazol- 2-yl]pyrrolidine-1- carbonyl]-2-methyl- propyl]carbamate | (36%) | | (4H, m), 3.06-3.14 (4H, m), 3.53 (3H, s), 3.64 (2H, s), 3.75-3.85 (2H, b), 4.05 (1H, m), 5.04- 5.12 (1H, m), 7.26-7.99 (13H, m), 8.22 (1H, s), 10.35 (1H, s) |
| Example 29, | Methyl N-[(1S)-2- | Yellow | m/z | 1.20 (3H, d, J = 6.95 Hz), |
| R= | [(2S)-2-[5-[4-[3-[[4- | oil/solid | 685 | 1.60-2.20 (4H, b), 2.80- |
| 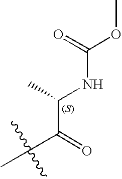 | [(1,1-dioxo-1,4- thiazinan-4- yl)methyl]phenyl] carbamoyl]phenyl] phenyl]-1H-imidazol- 2-yl]pyrrolidin-1-yl]- 1-methyl-2-oxo- ethyl]carbamate | (39%) | | 2.91 (4H, m), 3.06-3.13 (4H, m), 3.52 (3H, s), 3.57-3.82 (3H, s and b), 3.95-4.23 (1H, b), 4.24- 4.42 (1H, m), 5.03-5.16 (1H, m), 7.28-8.01 (13H, m), 8.22 (1H, s), 10.35 (1H, s), 13.37-13.96 (1H, b) |
| Example 30, | Methyl N-[(1S)-2- | Yellow | m/z | 1.80-2.06 (4H, b), 2.82- |
| R = | [(2S)-2-[5-[4-[3-[[4- | oil | 747 | 2.90, 4H, m), 3.06-3.16 |
| 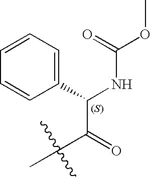 | [(1,1-dioxo-1,4- thiazinan-4- yl)methyl]phenyl] carbamoyl]phenyl] phenyl]-1H-imidazol- 2-yl]pyrrolidin-1-yl]- 2-oxo-1-phenyl- ethyl]carbamate | (47%) | | (4H, m), 3.50-3.56 (3H, m), 3.64 (2H, s), 4.01- 4.16 (2H, b), 5.03-5.15 (1H, m), 5.45-5.54 (1H, m), 7.28-8.00 (18H, m), 8.23 (1H, s), 10.36 (1H, s), 13.40-13.90 (1H, b) |
| Example 31, | Methyl N-[(1R)-2- | Yellow | m/z | 1.81-2.20 (4H, b), 2.82- |
| R = | [(2S)-2-[5-[4-[3-[[4- | oil | 747 | 2.92 (4H, m), 3.06-3.16 |
| 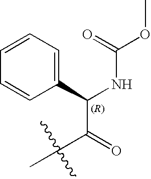 | [(1,1-dioxo-1,4- thiazinan-4- yl)methyl]phenyl] carbamoyl]phenyl] phenyl]-1H-imidazol- 2-yl]pyrrolidin-1-yl]- 2-oxo-1-phenyl- ethyl]carbamate | (59%) | | (4H, m), 3.51-3.56 (3H, m), 3.64 (2H, s), 4.01- 4.16 (2H, b), 5.03-5.17 (1H, m), 5.30-5.56 (1H, m), 7.28-7.80 (18H, m), 8.23 (1H, s), 10.36 (1H, s), 13.40-13.90 (1H, b) |
| Example 32, | 3-[4-[2-[(2S)-1- | Yellow | m/z | 0.93-1.12 (6H, m), 1.79- |
| R = | [(2R)-2- | solid | 745 | 2.20 (4H, b), 2.75-2.97 |
| 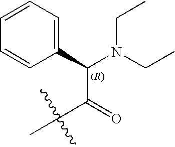 | (diethylamino)-2- phenyl- acetyl]pyrrolidin-2- yl]-1H-imidazol-5- yl]phenyl]-N-[4- [(1,1-dioxo-1,4- thiazinan-4- yl)methyl]phenyl] benzamide | (28%) | | (8H, m), 3.06-3.12 (4H, m), 3.64 (2H, s), 4.0-4.16 (1H, b), 5.01-5.25 (1H, m), 7.27-7.28 (18H, m), 8.23 (1H, s), 10.36 (1H, s) |

The following examples were prepared by the method of Example 4 using N-[4-[(1,1-dioxo-1,4-thiazinan-4-yl)methyl]phenyl]-4-[3-[2-[(2S)-pyrrolidin-2-yl]-1H-imidazol-5-yl]phenyl]benzamide (IId) and the appropriate carboxylic acid.

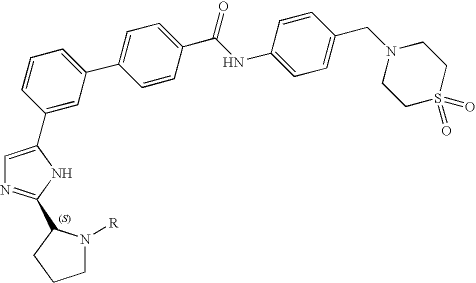

| | | | LC- | |
|---|---|---|---|---|
| R group | Name | Yield | MS | 1H NMR |
| Example 33, | Methyl N-[(1S)-1- | Pale | m/z | 0.71-0.90 (6H, m), 1.68- |
| R = | [(2S)-2-[5-[3-[4-[[4- | yellow | 713 | 2.17 (4H, b), 2.74-2.85 |
| 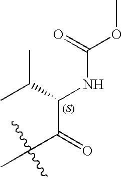 | [(1,1-dioxo-1,4- thiazinan-4- yl)methyl]phenyl] carbamoyl]phenyl] phenyl]-1H-imidazol- 2-yl]pyrrolidine-1- carbonyl]-2-methyl- propyl]carbamate | solid (40%) | | (4H, m), 2.98-3.10 (4H, m), 3.46 (3H, s), 3.57 (2H, s), 3.67-3.80 (2H, b), 3.93-4.06 (1H, m), 4.97-5.07 (1H, m), 7.19- 8.07 (15H, m), 10.24 (1H, s), 11.76 (0.5H, s) |
| Example 34, | Methyl N-[(1S)-2- | White | m/z | 1.14-1.25 (6H, m), 1.80- |
| R = | [(2S)-2-[5-[3-[4-[[4- | solid | 685 | 2.23 (4H, b), 2.81-2.93 |
| 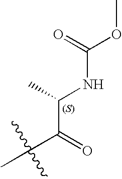 | [(1,1-dioxo-1,4- thiazinan-4- yl)methyl]phenyl] carbamoyl]phenyl] phenyl]-1H-imidazol- 2-yl]pyrrolidin-1-yl]- 1-methyl-2-oxo- ethyl]carbamate | (25%) | | (4H, m), 3.05-3.19 (4H, m), 3.52 (3H, s), 3.63 (2H, s), 3.63-3.84 (2H, b), 4.28-.43 (1H, m), 5.04-5.06 (1H, m), 7.25.8.14 (14H, m), 10.31 (1H, s), 11.76 and 12.08 (1H, 2 x s) |
| Example 35, | Methyl N-[(1S)-1- | White | m/z | 0.96 (9H, s), 1.80-2.23 |
| R = | [(2S)-2-[5-[3-[4-[[4- | solid | 727 | (4H, b), 2.80-2.91 (4H, |
| 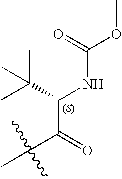 | [(1,1-dioxo-1,4- thiazinan-4- yl)methyl]phenyl] carbamoyl]phenyl] phenyl]-1H-imidazol- 2-yl]pyrrolidine-1- carbonyl]-2,2- dimethyl- propyl]carbamate | (39%) | | m), 3.06-3.15 (4H, m), 3.55 (3H, s), 3.64 (2H, s), 3.74-3.86 (2H, b), 4.18- 4.28 (1H, m), 5.04-5.13 (1H, m), 7.04-8.11 (14H, m), 10.30 (1H, s), 11.83, 12.00 and 12.31 (1H, 3 x s) |
| Example 36, | Methyl N-[(1S)-2- | Oil | m/z | 1.74-2.21 (4H, b), 2.82- |
| R = | [(2S)-2-[5-[3-[4-[[4- | (40%) | 747 | 2.91 (4H, m), 3.05-3.16 |
| 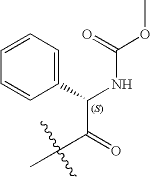 | [(1,1-dioxo-1,4- thiazinan-4- yl)methyl]phenyl] carbamoyl]phenyl] phenyl]-1H-imidazol- 2-yl]pyrrolidin-1-yl]- 2-oxo-1-phenyl- ethyl]carbamate | | | (4H, m), 3.35-3.41 (1H, m), 3.48-3.55 (3H, m), 3.64 (3H, s), 5.03-5.17 (1H, m), 5.39-5.55 (1H, m), 6.90-8.10 (19H, m), 10.31 (1H, s), 11.60- 12.00 (0.5H, b) |
| Example 37, | Methyl N-[(1R)-2- | Oil | m/z | 1.71-2.17 (4H, b), 2.75- |
| R = | [(2S)-2-[5-[3-[4-[[4- | (42%) | 747 | 2.85 (4H, m), 2.98-3.15 |
| 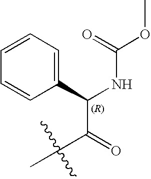 | [(1,1-dioxo-1,4- thiazinan-4- yl)methyl]phenyl] carbamoyl]phenyl] phenyl]-1H-imidazol- 2-yl]pyrrolidin-1-yl]- 2-oxo-1-phenyl- ethyl]carbamate | | | (4H, m), 3.37-3.51 (5H, m), 3.56 (2H, s), 4.56- 5.15 (1H, m), 5,30-5.48 (1H, m), 6.82-8.09 (19H, m), 10.23 (1H, s), 11.68- 12.00 (1H, b) |
| Example 38, | 4-[3-[2-[(2S)-1- | White | m/z | 0.83-0.96 (6H, m), 1.79- |
| R = | [(2R)-2- | solid | 745 | 2.20 (4H, b), 2.55-2.72 |
| 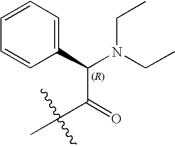 | (diethylamino)-2- phenyl- acetyl]pyrrolidin-2- yl]-1H-imidazol-5- yl]phenyl]-N-[4- [(1,1-dioxo-1,4- thiazinan-4- yl)methyl]phenyl] benzamide | (40%) | | (4H, m), 3.08-3.15 (4H, m), 3.46-3.5 (1H, b), 3.64 (2H, s), 3.89-4.01 (1H, b), 4.63, 4.71 (1H, 2 x s), 5.03-5.01, 5.51-5.60 (1H, 2 x b), 6.92-8.13 (18H, m), 10.31 (1H, s), 11.79- 12.11 (1H, b) |
| Example 39, | 4-[3-[2-[(2S)-1- | White | m/z | 1.77-2.16 (4H, m), 2.19 |
| R = | [(2R)-2- | solid | 717 | (6H, s), 2.80-2.90 (4H, |
| 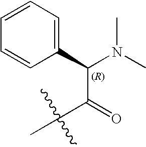 | (dimethylamino)-2- phenyl- acetyl]pyrrolidin-2- yl]-1H-imidazol-5- yl]phenyl]-N-[4- [(1,1-dioxo-1,4- thiazinan-4- yl)methyl]phenyl] benzamide | (40%) | | m), 3.06-3.17 (4H, m), 3.43-3.56 (1H, m), 3.63- (2H, s), 3.94-4.04 (1H, m), 4.16, 4.39 (1H, 2 x s), 4.99-5.05, 5.45-5.58 (1H, 2 x b), 6.84-8.14 (18H, m), 10.31 (1H, s), 11.89-12.10 (0.5H, b) |

The following examples were prepared by the method of Example 4 using N-[4-[(4-propylsulfonylpiperazin-1-yl)methyl]phenyl]-4-[4-[2-[(2S)-pyrrolidin-2-yl]-1H-imidazol-5-yl]phenyl]benzamide (lie) and the appropriate carboxylic acid.

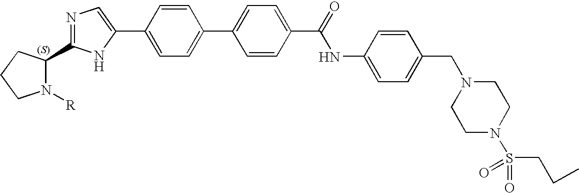

| | | | LC- | |
|---|---|---|---|---|
| R group | Name | Yield | MS | 1H NMR |
| Example 40, | Methyl N-[(1S)-2- | Yellow | m/z | 0.80-0.92 (6H, m), 0.97 |
| R = | methyl-1-[(2S)-2-[5- | solid | 770 | (3H, t, J = 7.58 Hz), 1.61- |
| 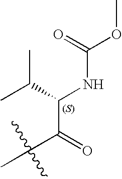 | [4-[4-[[4-[(4- propylsulfonyl piperazin-1- yl)methyl]phenyl] carbamoyl]phenyl] phenyl]-1H-imidazol- 2-yl]pyrrolidine-1- carbonyl]propyl] carbamate | (21%) | | 1.76 (2H, m), 1.81-2.23 (5H, b), 2.39-2.46 (4H, m), 2.95-3.04 (2H, m), 3.11-3.19 (4H, m), 3.48 (3H, s), 3.53 (2H, s), 3.74-3.85 (2H, b), 4.02- 4.11 (1H, m), 5.03-5.11 (1H, m), 7.24-8.09 (14H, m), 10.26 (1H, s), 11.82 (1H, s) |
| Example 41, | Methyl N-[(1S)-1- | Oil | m/z | 0.97 (3H, t, J = 7.42 Hz), |
| R = | methyl-2-oxo-2- | (42%) | 742 | 1.21 (3H, d, J = 6.95 Hz), |
| 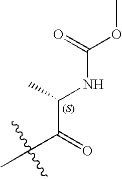 | [(2S)-2-[5-[4-[4-[[4- [(4-propylsulfonyl piperazin-1- yl)methyl]phenyl] carbamoyl]phenyl] phenyl]-1H-imidazol- 2-yl]pyrrolidin-1- yl]ethyl]carbamate | | | 1.59-1.78 (2H, m), 1.80- 2.35 (4H, b), 2.38-2.46 (4H, m), 2.95-3.04 (2H, m), 3.10-3.20 (4H, m), 3.42-3.54 (5H, m), 3.42- 3.54 (2H, b), 4.05-4.19 (1H, b), 4.29-4.41 (1H, m), 5.03-5.16 (1H, m), 7.24-8.08 (14H, m), 10.27 (1H, s), 11.68-11.83 (1H, b) |
| Example 42, | Methyl N-[(1S)-2- | Yellow | m/z | 0.97 (3H, t, J = 7.42 Hz), |
| R = | oxo-1-phenyl-2- | solid | 804 | 1.60-1.75 (2H, m), 1.81- |
| 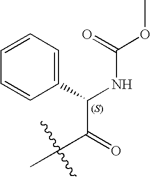 | [(2S)-2-[5-[4-[4-[[4- [(4-propylsulfonyl piperazin-1- yl)methyl]phenyl] carbamoyl]phenyl] phenyl]-1H-imidazol- 2-yl]pyrrolidin-1- yl]ethyl]carbamate | (78%) | | 2.06 (4H, b), 2.36-2.46 (4H, m), 2.95-3.04 (2H, m), 3.09-3.20 (4H, m) 3.46-3.57 (7H, m), 5.01- 5.17 (1H, m), 5.44-5.55 (1H, m), 7.26-8.10 (19H, m), 10.27 (1H, s), 11.77- 12.12 (1H, b) |
| Example 43, | Methyl N-[(1R)-2- | Oil | m/z | 0.97 (3H, t, J = 7.42 Hz), |
| R = | oxo-1-phenyl-2- | (48%) | 804 | 1.60-1.75 (2H, m), 1.81- |
| 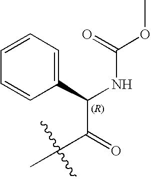 | [(2S)-2-[5-[4-[4-[[4- [(4-propylsulfonyl piperazin-1- yl)methyl]phenyl] carbamoyl]phenyl] phenyl]-1H-imidazol- 2-yl]pyrrolidin-1- yl]ethyl]carbamate | | | 2.06 (4H, b), 2.36-2.47 (4H, m), 2.95-3.04 (2H, m), 3.11-3.19 (4H, m), 3.46-3.57 (7H, m), 5.01- 5.17 (1H, m), 5.43-5.56 (1H, m), 7.26-8.10 (19H, m), 10.27 (1H, s), 11.74- 11.91 (1H, b) |
| Example 44, | 4-[4-[2-[(2S)-1- | Pale | m/z | 0.86-1.03 (6H, m), 1.58- |
| R = | [(2R)-2- | white | 802 | 1.76 (2H, m), 1.79-2.19 |
| 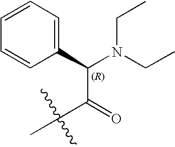 | (diethylamino)-2- phenyl- acetyl]pyrrolidin-2- yl]-1H-imidazol-5- yl]phenyl]-N-[4-[(4- propylsulfonyl piperazin-1- yl)methyl]phenyl] benzamide | solid (53%) | | (4H, b), 2.36-2.48 (4H, m), 2.57-2.81 (4H, b), 2.95-3.04 (2H, m), 3.12- 3.22 (4H, m) 3.31-3.47 (2H, b), 3.48 (2H, s), 3.92-4.05 (1H, b), 4.84- 5.00, 5.01-5.10 (1H, b + m), 6.91- 8.13 (18H, m), 10.27 (1H, s), 11.80- 12.12 (1H, b) |
| Example 45, | 4-[4-[2-[(2S)-1- | White | m/z | 0.97 (3H, t, J = 7.42 Hz), |
| R = | [(2R)-2- | solid | 773 | 1.59-1.75 (2H, m), 1.78- |
| 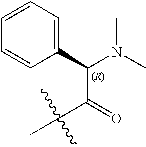 | (dimethylamino)-2- phenyl- acetyl]pyrrolidin-2- yl]-1H-imidazol-5- yl]phenyl]-N-[4-[(4- propylsulfonyl piperazin-1- yl)methyl]phenyl] benzamide | (57%) | | 2.15 (4H, b), 2.25-2.49 (10H, m), 2.95-3.04 (2H, m), 3.16-3.33 (4H, m), 3.36-3.43 (2H, b), 3.49 (2H, s), 3.95-4.15 (1H, b), 4.85-5.08 (1H, m), 6.87-8.12 (18H, m), 10.27 (1H, s), 11.63- 12.10 (1H, b) |

### EXAMPLE 46

### Methyl N-[(1S)-1-[(3R)-3-[5-[4-[4-[[4-[(1,1-dioxo-1,4-thiazinan-4-yl)methyl]phenyl]carbamoyl]phenyl]phenyl]-1H-imidazol-2-yl]morpholine-4-carbonyl]-2-methyl-propyl]carbamate

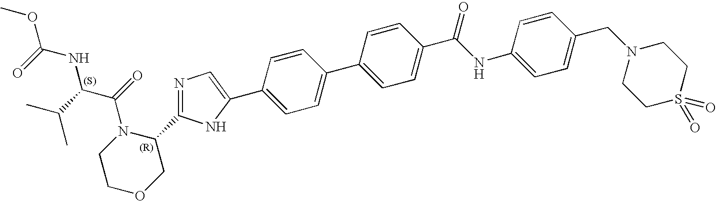

The title compound was produced using the method of Example 4 with N-[4-[(1,1-dioxo-1,4-thiazinan-4-yl)methyl]phenyl]-4-[4-[2-[(3R)-morpholin-3-yl]-1H-imidazol-5-yl]phenyl]benzamide (III) and (2S)-2-(methoxycarbonylamino)-3-methylbutanoic acid (A3).

Colourless solid (26 mg)

LC/MS m/z 726 (ES)

1H NMR (δ, CDCl3) 0.78-0.90 (dd, 2H, J=6.95 Hz), 0.97-1.17 (dd, 4H, J=6.63 Hz), 1.97 (septet, 1H), 2.72-2.82 (m, 1H), 2.82-2.99 (m, 8H), 3.40-3.72 (m, 9H), 3.84-3.97 (m, 0.9H), 4.26-4.51 (m, 1.8H), 4.91-5.04 (m, 1H), 5.54-5.69 (m, 1H), 7.16-7.24 (m, 3H), 7.33 (s, is 0.6H), 7.41 (d, 0.6H, J=8.21 Hz), 7.48 (d, 2H, J=8.21 Hz), 7.55-7.63 (m, 4.5H), 7.72-7.89 (m, 4H), 8.25 (s, 0.7H), 8.39 (s, 0.3H)

The following examples were prepared by the method of Example 4 using N-[4-[(1,1-dioxo-1,4-thiazinan-4-yl)methyl]phenyl]-4-[4-[2-[(3R)-morpholin-3-yl]-1H-imidazol-5-yl]phenyl]benzamide (IIf) and the appropriate carboxylic acid.

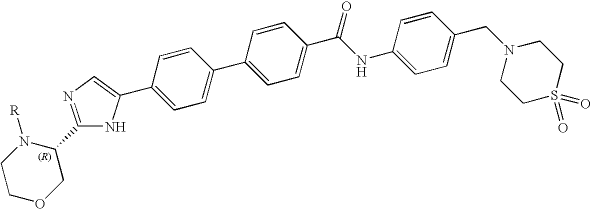

| | | | LC- | |
|---|---|---|---|---|
| R group | Name | Yield | MS | 1H NMR |
| Example 47, | Methyl N-[(1S)-2- | Colour- | m/7 | 2.82-2.86 (m, 4H) 2.87- |
| R = | [(3R)-3-[5-[4-[4-[[4- | less | 763 | 3.00 (m, 4H) 3.20-3.24 |
| 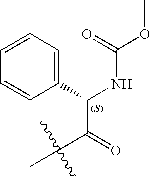 | [(1,1-dioxo-1,4- thiazinan-4- yl)methyl]phenyl] carbamoyl]phenyl] phenyl]-1H-imidazol- 2-yl]morpholin-4-yl]- 2-oxo-1-phenyl- ethyl]carbamate | solid (37 mg) | | (m, 0.5H) 3.25-3.39 (m, 0.5H) 3.41-3.48 (m, 1H) 3.53 (s, 2H) 3.58-3.67 (s + m, 3H) 3.70 (s, 1H) 3.77-3.92 (m, 1H) 4.36- 4.57 (m, 0.3H) 4.68-4.98 (m, 0.8H) 5.41-5.46 (m, 0.4H) 5.54-5.57 (m, 0.3H) 5.61-5.74 (m, 0.9H) 5.78-5.83 (m, 0.4H) 7.16-7.27 (m, 4H) 7.30-7.42 (m, 4H) 7.44- 7.53 (m, 1.5H) 7.53- 7.656 (m, 5H) 7.70-7.80 (m, 1.5H) 7.81-7.93 (m, 2H) 8.11 (s, 0.4H) 8.20 (s, 0.2H) 8.25 (s, 0.4H) |
| Example 48, | tert-Butyl N-[(1S)-2- | Beige | m/z | 1.32-1.40 (brs, 6.5H) |
| R = | [(3R)-3-[5-[4-[4-[[4- | solid | 805 | 1.44 (s, 2.5H) 2.71 (s, |
| 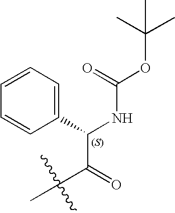 | [(1,1-dioxo-1,4- thiazinan-4- yl)methyl]phenyl] carbamoyl]phenyl] phenyl]-1H-imidazol- 2-yl]morpholin-4-yl]- 2-oxo-1-phenyl- ethyl]carbamate | (47 mg) | | 5H) 2.79 (d, 2H, J = 0.63 Hz) 2.86-2.89 (s + m, 4H) 2.91-2.97 (m, 2.25H) 3.2-3.5 (br, 0.75H) 3.52 (s, 2H) 3.62-3.7 (br, 0.5H) 4.5-4.9 (br, 0.25H) 5.26-5.72 (brm, 1H) 7.15-7.23 (m, 2H) 7.24- 7.29 (br, 1H) 7.30-7.40 (m, 4.5H) 7.48 and 7.51 (2xd, 2H, J = 1.89 Hz) 7.53-7.68 (m, 4.5H) 7.73- 7.82 (m, 2.5H) 8.58 (s, 0.4H) 8.64 (s, 0.6H) |
| Example 49, | 4-[4-[2-[(3R)-4- | Colour- | m/z | 2.29 (s, 3H), 2.35 (s, 5H), |
| R = | [(2R)-2- | less | 733 | 2.90-3.04 (m, 8H), 3.59 |
| 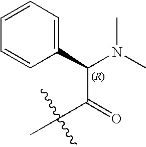 | (dimethylamino)-2- phenyl- acetyl]morpholin-3- yl]-1H-imidazol-5- yl]phenyl]-N-[4- [(1,1-dioxo-1,4- thiazinan-4- yl)methyl]phenyl] benzamide | solid (53 mg) | | (brs, 3H), 4.19 (s, 0.3H), 4.40 (brs, 0.8H), 4.59 (brd, 0.9H), 5.64 (brs, 1H), 7.16 (s, 1H), 7.25 (d, 2H, J = 8.53 Hz), 7.33- 7.47 (m, 6H), 7.52 (d, 2H, J = 8.21 Hz), 7.60 (d, 2H, J = 7.58 Hz), 7.68 (d, 4H, J = 8.53 Hz), 7.91 (d, 2H, J = 7.89 Hz), 8.58 (s, 1H) |

The following examples were prepared by the method of example 4 using N-[4-[(1,1-dioxo-1,4-thiazinan-4-yl)methyl]phenyl]-4-[4-[2-[(2S)-2-piperidyl]-1H-imidazol-5-yl]phenyl]benzamide (IIg) and the appropriate carboxylic acid.

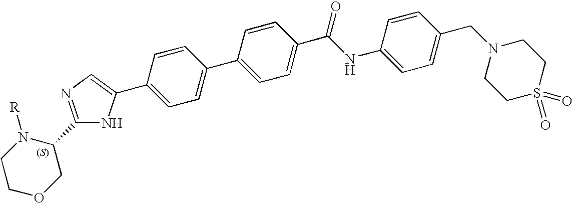

| | | | LC- | |
|---|---|---|---|---|
| R group | Name | Yield | MS | 1H NMR |
| Example 50, | methyl N-[(1S)-1- | Off-white | m/z | 0.95 and 0.87 (2xd, 1H, |
| R = | [(2S)-2-[5-[4-[4- | gum | 727 | J = 6.63 and 6.95 Hz |
| 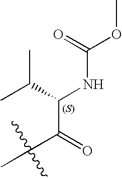 | [[4-[(1,1-dioxo-1,4- thiazinan-4- yl)methyl]phenyl] carbamoyl]phenyl] phenyl]-1H- imidazol-2- yl]piperidine-1- carbonyl]-2- methyl- propyl]carbamate | (61 mg) | | respectively), 1.13 (d, 5H, J = 6.63 Hz), 1.42- 1.92 (m, 4H), 2.05 (septet, 1H), 2.21-2.38 (m, 1H), 2.40-2.55 (m, 1H), 2.80-2.92 (m, 1H), 2.95-3.13 (m, 8H), 3.63 (s, 2H), 3.71-3.79 (m, 3H), 4.43 (t, 0.5H, J = 7.89 Hz), 4.56-4.65 (m, 1H), 5.32-5.38 (br, 1H), 5.84-5.94 and 5.64-5.76 (2xm, 1H), 7.32 (d, 2H, J = 8.21 Hz), 7.41 (s, 1H), 7.61 (d, 2H, J = 8.53 Hz), 7.65-7.74 (m, 4H), 7.87 (d, 2H, J = 8.21 Hz), 7.96 (d, 2H, J = 8.53 Hz), 8.30 (brs, 1H) |
| Example 51, | Methyl N-[(1S)-2- | Colour- | m/z | 1.18-1.89 (m, 6H) 2.36- |
| R = | [(2S)-2-[5-[4-[4- | less solid | 761 | 2.72 (m, 1H) 2.87-2.90 |
| 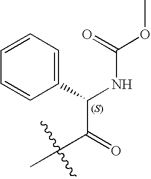 | [[4-[(1,1-dioxo-1,4- thiazinan-4- yl)methyl]phenyl] carbamoyl]phenyl] phenyl]-1H- imidazol-2-yl]-1- piperidyl]-2-oxo-1- phenyl- ethyl]carbamate | (53 mg) | | (m, 4H) 2.92-2.99 (m, 4H) 3.55 (s, 2H) 3.45- 3.68 (m, 3H) 4.55-4.66 (m, 0.3H) 5.06-5.12 (m, 0.3H) 5.48-5.67 (m, 0.7H) 5.74-5.88 (m, 0.5H) 5.92-6.03 (m, 0.5H) 6.25-6.30 (m, 1H) 7.17-7.26 (m, 3.5H) 7.27-7.40 (m, 4.5H) 7.44-7.52 (m, 1.5H) 7.53-7.64 (m, 5H) 7.70-7.81 (m, 1.5H) 7.82-7.88 (m, 2H) 8.24 (m, 0.6H) 8.34 (s, 0.3H) |
| Example 52, | tert-Butyl N-[(1S)- | Colourless | m/z | 1.19-1.64 (m + 2xs, |
| R = | 2-[(2S)-2-[5-[4-[4- | solid | 803 | 15H) 2.67 (m, 0.5H) |
| 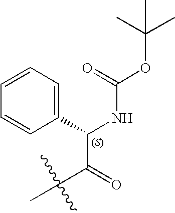 | [[4-[(1,1-dioxo-1,4- thiazinan-4- yl)methyl]phenyl] carbamoyl]phenyl] phenyl]-1H- imidazol-2-yl]-1- piperidyl]-2-oxo-1- phenyl- ethyl]carbamate | (92 mg) | | 2.73 (m, 1.5H) 2.81- 2.92 (m + s, 5H) 3.04- 3.14 (m, 4H) 3.64 (s, 2H) 3.68-3.78 (m, 0.5H) 4.35-4.44 (m, 0.3H) 5.16-5.20 (m, 0.2H) 5.60-5.81 (m, 1H) 7.27-7.50 (m, 7H) 7.76-7.94 (m, 9H) 8.02- 8.08 (m, 2H) 10.28 (s, 1H) |
| Example 53, | 4-[4-[2-[(2S)-1- | Off-white | m/z | 1.41-1.82 (m, 4H) 2.28- |
| R = | [(2R)-2- | gum | 731 | 2.53 (m, 2H) 2.33 (s, |
| 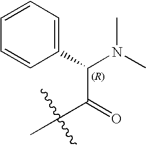 | (dimethylamino)-2- phenyl-acetyl]-2- piperidyl]-1H- imidazol-5- yl]phenyl]-N-[4- [(1,1-dioxo-1,4- thiazinan-4- yl)methyl]phenyl] benzamide | (63 mg) | | 6H) 2.94-3.01 (m, 4.5H) 3.02-3.29 (m, 5.5H) 3.63 (s, 2H) 3.90-4.02 (m, 0.6H) 4.42 (s, 0.6H) 5.84- 5.92 (m, 0.7H) 7.23- 7.34 (m, 4H) 7.38-7.47 (m, 4H) 7.58-7.73 (m, 7H) 7.74-7.83 (m, 1H) 7.93-7.99 (m, 2H) 8.24- 8.28 (m, 1H) |

### EXAMPLE 54

### Methyl N-[(1S)-1-[(2S)-2-[5-[4-[4-[[4-[(1,1-dioxo-1,4-thiazinan-4-yl)methyl]phenyl]carbamoyl]phenyl]-3-methyl-phenyl]-1H-imidazol-2-yl]pyrrolidine-1-carbonyl]-2-methyl-propyl]carbamate

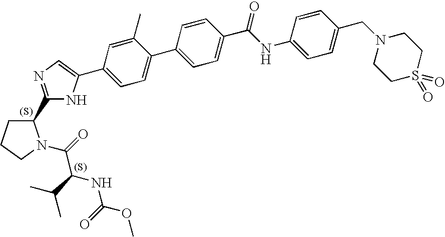

This was prepared by the method of example 4 using N-[4-[(1,1-dioxo-1,4-thiazinan-4-yl)methyl]phenyl]-4-[2-methyl-4-[2-[(2S)-pyrrolidin-2-yl]-1H-imidazol-5-yl]phenyl]benzamide (IIh) and the appropriate carboxylic acid (A3).

Yellow solid (33%)

LC-MS m/z 727, 725

¹H NMR (δ, d₆-DMSO) 0.77-0.96 (6H, m), 1.80-2.22 (5H, b), 2.28 (3H, s), 2.79-2.92 (4H, b), 3.04-3.20 (4H, b), 3.53 (3H, s), 3.63 (2H, s), 3.72-3.86 (1H, b), 4.0-4.16 (1H, b), 5.03-5.12 (1H, b), 7.17-8.07 (14H, m), 10.30 (1H, s)

### PHARMACOLOGICAL EXAMPLES

### 1b Replicon Assay

### Cells Used:

HCV replicon cells Huh 9B (ReBlikon), containing the firefly luciferase-ubiquitin-neomycin phosphotransferase fusion protein and EMCV-IRES driven HCV polyprotein with cell culture adaptive mutations.

### Cell Culture Conditions:

Cells were cultured at 37° C./5% CO₂ and split twice a week. G418 at 0.5 mg/ml was added to the culture medium but not the assay medium.

The culture medium consisted of DMEM with 4500 g/l glucose and Glutamax (Gibco 61965-026) supplemented with 1× non-essential amino acids (Invitrogen 11140-035), 0.5 mg/ml G418 (Invitrogen 10131-027) and 10% Australian foetal calf serum (Invitrogen 10099-141).

### Assay Procedure:

Replicon cells were trypsinised and counted. Cells were diluted to 100,000 cells/ml and 100 μl used to seed one opaque white 96-well plate (for the replicon assay) and one flat-bottomed clear plate (for the tox assay) for every five compounds to be tested for IC₅₀. Wells G12 and H12 were left empty in the clear plate as the blank. Plates were then incubated at 37° C./5% CO₂ for 24 h.

On the following day compound dilutions were prepared in medium at twice their desired final concentration in a clear round bottomed plate. All dilutions have a final DMSO concentration of 1%.

Controls and compounds were transferred from the dilution plates to the assay plates (containing the cells) at 100 μl/well in duplicate wells.

Exception: no compound was added to wells A1 and A2 of either plate and 100 μl of 1% DMSO was added to these instead. Plates were then incubated at 37° C./5% CO₂ for 72 h.

At the end of the incubation time, the cells in the white plate were washed in PBS (100 μL per well) and dried by tapping before addition of 204 per well of lysis buffer (25 mM tris-phosphate, 8 mM MgCl₂, 1 mM DTT, 1% Triton X-100, 15% glycerol; pH to 7.8 using KH₂PO₄ prior to Triton X-100 and glycerol addition). Aliquots of substrate (23.5 mM beetle luciferin (Promega E1603), 26 mM ATP (Sigma O-2060) in 100 nM Tris buffer pH 7.8) were stored at −80° C. Prior to use, required amount of luciferin was thawed and diluted 1:50 in luciferase assay buffer (20 mM Tricine (Sigma T-0377), 1.07 mM magnesium carbonate hydroxide (Sigma M-5671), 0.1 mM EDTA (Sigma E-5134), 2.67 mM MgSO₄(BDH 101514Y), 33.3 mM dithiothreitol (Sigma 150460) pH 7.8).

After 5-60 min incubation in lysis buffer at room temperature, a plate was inserted into the luminometer and 100 μl luciferase assay reagent was added by the injector of the luminometer. The signal was measured using a 1 second delay followed by a 4 second measurement programme. The IC₅₀, the concentration of the drug required for reducing the replicon level by 50% in relation to the untreated cell control value, was calculated from the plot of the percentage reduction of the luciferase activity vs. drug concentration.

The clear plate was stained with 100 μA 0.5% methylene blue in 50% ethanol at room temperature for 1 h, followed by solvation of the absorbed methylene blue in 100 μl per well of 1% lauroylsarcosine. Absorbance of the plate was measured on a microplate spectrophotometer (Molecular Devices) and the absorbance for each concentration of compound expressed as a proportion of the relative DMSO control. The TD₅₀, the is concentration of drug required to reduce the total cell area by 50% relative to the DMSO controls, was calculated by plotting the absorbance at 620 nm after background substraction against drug concentration.

When tested in the above screen, the compounds of the Examples gave IC₅₀ values for reduction of the replicon level of less than 11.1M (micromolar), indicating that the compounds of the invention are expected to possess useful therapeutic properties. The results obtained are shown in the following Table.

### 1b Replicon Assay Results:

| Example | HCV IC₅₀ 1b Replicon | pIC₅₀ | HCV TD₅₀ Replicon |
|---|---|---|---|
| No. | (uM) | 1b Replicon | (uM) |
| 1 | 0.00001 | 10.86 | >25 |
| 2 | 0.00002 | 10.69 | >25 |
| 3 | 0.00034 | 9.47 | >25 |
| 4 | 0.00118 | 8.93 | 22.44 |
| 5 | 0.00073 | 9.13 | 3.9 |
| 6 | 0.00004 | 10.38 | >25 |
| 7 | 0.00005 | 10.33 | >25 |
| 8 | 0.00005 | 10.33 | >25 |
| 9 | 0.00010 | 10.00 | >25 |
| 10 | 0.06524 | 7.19 | NT |
| 11 | 0.00113 | 8.95 | >21 |
| 12 | 0.10592 | 6.98 | NT |
| 13 | 0.04216 | 7.38 | >25 |
| 14 | 0.00036 | 9.45 | >25 |
| 15 | 0.00384 | 8.42 | >25 |
| 16 | 0.00013 | 9.88 | >25 |
| 17 | 0.00030 | 9.52 | 18.4 |
| 18 | 0.12552 | 6.90 | >25 |
| 19 | 0.04323 | 7.36 | 21.4 |
| 20 | 0.31888 | 6.50 | NT |
| 21 | 0.41821 | 6.38 | NT |
| 22 | 0.26575 | 6.58 | NT |
| 23 | 0.10877 | 6.96 | NT |
| 24 | 0.10136 | 6.99 | NT |
| 25 | 0.07788 | 7.11 | NT |
| 26 | 0.05574 | 7.25 | NT |
| 27 | 0.01529 | 7.82 | >25 |
| 28 | 0.00732 | 8.14 | >25 |
| 29 | 0.03341 | 7.48 | >25 |
| 30 | 0.00120 | 8.92 | >25 |
| 31 | 0.00091 | 9.04 | >25 |
| 32 | 0.00021 | 9.68 | >25 |
| 33 | 0.15905 | 6.80 | NT |
| 34 | 0.25217 | 6.60 | NT |
| 35 | 0.12901 | 6.89 | NT |
| 36 | 0.01556 | 7.81 | NT |
| 37 | 0.02147 | 7.67 | NT |
| 38 | 0.00287 | 8.54 | NT |
| 39 | 0.00481 | 8.32 | NT |
| 40 | 0.00010 | 10.00 | NT |
| 41 | 0.00115 | 8.94 | NT |
| 42 | 0.00005 | 10.27 | NT |
| 43 | 0.00005 | 10.26 | NT |
| 44 | 0.00001 | 10.84 | NT |
| 45 | 0.00003 | 10.58 | NT |
| 46 | 0.00071 | 9.15 | >25 |
| 47 | 0.00028 | 9.56 | >25 |
| 48 | 0.00051 | 9.29 | >25 |
| 49 | 0.00088 | 9.05 | 19.7 |
| 50 | 0.00298 | 8.53 | NT |
| 51 | 0.00079 | 9.10 | NT |
| 52 | 0.00041 | 9.39 | NT |
| 53 | 0.00082 | 9.09 | NT |
| 54 | 0.00021 | 9.68 | >25 |

pIC₅₀ = −log10 (IC₅₀ in uM)
NT = Not Tested

### 1a Replicon Assay

### Cells Used:

HCV genotype la replicon cells Htat2ANeo (University of Texas), containing neomycin phosphotransferase fusion protein and EMCV-IRES driven HCV polyprotein with cell culture adaptive mutations, and control cells, Et₂AN.

### Cell Culture Conditions:

Cells were cultured at 37° C./5% CO₂ and split twice a week. G418 at 0.5 mg/ml and Blastocidin at 2 mg/ml were added to the culture medium but not the assay medium.

The culture medium consisted of DMEM with 4500 g/l glucose and Glutamax (Gibco 61965-026) supplemented with 1× non-essential amino acids (Invitrogen 11140-035), 0.5 mg/ml G418 (Invitrogen 10131-027), 2 mg/ml Blastocidin (PAA) and 10% is Australian foetal calf serum (Invitrogen 10099-141).

### Assay Procedure:

Replicon and control cells were trypsinised and counted. Replicon cells were diluted to 90,000 cells/ml and 100 μl of this used to seed columns 2-4, 6-9 and 10-12 of a black, clear bottom 96-well plate for every three compounds to be tested for IC₅₀. Control cells were diluted to 60,000 cells/ml and 100 μl of this used to seed columns 1, 5 and 9 of the plate. Well H1 was left empty as the blank. Plates were then incubated at 37° C./5% CO₂ for 24 h.

On the following day compound dilutions were prepared in medium at twice their desired final concentration in a clear round bottomed plate. All dilutions had a final DMSO concentration of 1%.

Controls and compounds were transferred from the dilution plate to the assay plates (containing the cells) at 100 μl/well in quadruplicate wells, one well containing control cells and 3 wells containing replicon cells.

Exception: no compound was added to row H of the plate and 100 μl of 1% DMSO was added to these instead. Plates were then incubated at 37° C. with 5% CO₂ for 72 h. At the end of the incubation time, any reduction in cell viability was assayed and a cell-based enzyme linked immunosorbent assay (ELISA) was performed. The media/compound was removed from all wells and replaced with 100 μl/well serum-free media and 20 μl/well Cell Titre Blue (Promega G8081). Following incubation at 37° C./5% CO₂ for 2 h plates were read on a microplate fluorometer (Molecular Devices) using an excitation of 570 nm and an emission of 590 nm The TD50, the concentration of drug required to reduce 50% of Cell Titre Blue relative to the DMSO controls, was calculated by plotting the fluorescence at 590 nm after substraction of background against drug concentration.

The media/Cell Titre Blue was removed and plates washed in PBS and gently tapped dry before addition of 50 μL per well of 75% acetone/25% methanol mixture for 3 minutes. The fixative was then discarded and wells were washed with PBS before addition of 100 μl/well of blocking solution (2% non-fat dry milk and 0.05% Tween-20 in 0.85% NaCl). Plates were then incubated at 37° C. in a shaking incubator for 60 min. Blocking solution was discarded and 50 μl of mouse anti-NS5a antibody (Virostat 1877) at 1:100 dilution in blocking buffer was added to all wells. Plates were incubated at 37° C. in a shaking incubator for 90 min Antibody was then discarded and plates were washed 4 times by immersion in 0.85% NaCl/0.05% Tween-20. After washing plates were tapped dry gently and 50 μl of secondary antibody (Dako P0260 Rabbit anti-mouse horseradish peroxidase) at 1:1000 dilution in blocking buffer was added to the wells. Plates were incubated at 37° C. in a shaking incubator for 60 min. Antibody was discarded and plates were washed 6 times by immersion in 0.85% NaCl/0.05% Tween-20 and once in PBS. Finally, 50 μl of substrate (SigmaFast ortho-phenylene diamine dihydrochloride (OPD)) was added per well and the colorimetric reaction was allowed to proceed in the dark for 5 to 15 minutes depending on the strength or the signal. The reaction was stopped by addition of 25 μl/well of 20% sulphuric acid. Plates were read on the SpectraMax microplate reader at 490 nm fixed wavelength.

The IC50, the concentration of the drug required for reducing the replicon level by 50% in relation to the untreated cell control value, was calculated from the plot of the percentage reduction of the absorbance vs. drug concentration.

When tested in the above screen, the compounds of the Examples gave 1050 values for reduction of the replicon level of less than 10 μM (micromolar), indicating that the is compounds of the invention are expected to possess useful therapeutic properties. Specimen results are shown in the following Table.

### 1a Replicon Assay Results:

| Example No. | HCV IC₅₀ Elisa 1a (uM) | pIC₅₀ 1a Replicon |
|---|---|---|
| 1 | 0.04143 | 7.38 |
| 2 | 0.05602 | 7.25 |
| 3 | 0.00646 | 8.19 |
| 4 | 0.02591 | 7.59 |
| 5 | 0.03311 | 7.48 |
| 6 | 0.03331 | 7.48 |
| 7 | 0.03202 | 7.49 |
| 8 | 0.00475 | 8.32 |
| 9 | 0.01319 | 7.88 |
| 10 | 0.32031 | 6.49 |
| 11 | 0.01900 | 7.72 |
| 12 | 0.16378 | 6.79 |
| 14 | 0.01574 | 7.80 |
| 16 | 0.00839 | 8.08 |
| 20 | 1.47332 | 5.83 |
| 28 | 2.07569 | 5.68 |
| 29 | 7.61869 | 5.12 |
| 30 | 2.47767 | 5.61 |
| 31 | 2.69508 | 5.57 |
| 32 | 1.14949 | 5.94 |
| 38 | 0.31444 | 6.50 |
| 39 | 1.10260 | 5.96 |
| 40 | 0.04257 | 7.37 |
| 41 | 0.04702 | 7.33 |
| 42 | 0.09737 | 7.01 |
| 43 | 0.07769 | 7.11 |
| 44 | 0.05052 | 7.30 |
| 45 | 0.07951 | 7.10 |
| 46 | 0.01945 | 7.71 |
| 47 | 0.07331 | 7.13 |
| 48 | 0.05262 | 7.28 |
| 49 | 0.07784 | 7.11 |
| 50 | 0.02824 | 7.55 |
| 51 | 0.09497 | 7.02 |
| 52 | 0.18546 | 6.73 |
| 53 | 0.32373 | 6.49 |
| 54 | 0.00938 | 8.03 |

pIC₅₀ = −log10 (IC₅₀ in uM)

### Permeability Assay

Cells used: MDCK (Madin-Darby Canine Kidney) cells, ATCC collection # CCL-34, are used to model the intestinal barrier.
Cell culture conditions: cells were cultured at 37° C. in a 5% CO₂ environment and split twice a week on seeding at 4×10⁵ cells/flask (75 cm²) on day 1 and 2×10⁵ cells/flask on day 4. The culture medium consisted of MEM+Earle's-L Glutamine (Gibco #21090-022) supplemented with 10% Australian Fetal Calf Serum (Sigma #F6178), 2 mM L-Glutamine (Gibco #25030-024) and 1× Non Essential Amino Acids (Gibco #11140-035)

### Assay Procedure:

On day 1, a 24-well plate (Sarstedt, #83.1836.300) was filled with individual 3 lam pore membrane inserts (Millipore, #PITP 012 50). Each plate allows the testing of a cocktail of 3 control compounds and 11 test compounds in duplicate. The wells (outside the inserts) were filled with 500 μl of culture medium.

A flask of cells was trypsinised and a cell count carried out. Cells were diluted to 1.2×10⁵ cells/ml (1×10⁵ cells/cm²) and 500 μl dispensed in each insert on the 24-well plate. The plate was incubated at 37° C. in a 5% CO₂ environment for 48 hours.

On day 3, the culture medium was removed from the wells, then the inserts and is replaced with fresh culture medium in the wells, then the inserts (500111 per well and insert) The plate was incubated at 37° C. in a 5% CO₂ environment for 24 hours.

On day 4, the controls cocktail and test compounds solutions were made up in HBSS buffer (Hank's Balanced Salt Solution, Gibco #14025-050) at 1011M. The final controls and test compounds concentration in the assay was 10 μM, and DMSO concentration maintained at 0.1% (0.3% for the controls cocktail). The controls cocktail was made up of Atenolol (Sigma # A-7655), Dexamethasone (Sigma #D-1756) and Propranolol (Sigma # P-0884).

A 24-well plate was filled with 500 μl of HBSS buffer per well (assay plate) The culture medium was removed from the wells and inserts. The inserts were washed three times with approximately 500 μl of HBSS buffer. The inserts were transferred to the assay plate. Controls cocktail and test compounds solutions were dispensed inside the inserts (500 μl per insert), in duplicates. The assay plate was incubated at 37° C. in a 5% CO₂ environment for 2 hours.

After 2 hours, the inserts were removed from the assay plate and transferred into a new 24-well plate (wash plate) The assay plate containing the receiver solutions was left aside for later sampling. To measure the mass balance, 150 μl were sampled from each insert (donor solutions) and dispensed into 150 μl of HPLC-grade Acetonitrile (Fisher Scientific) containing an internal standard and 0.05% formic acid.

The donor solutions were aspirated and discarded from each insert, and the inserts washed once with approximately 500 μl of HBSS buffer. A 24-well plate was filled with 500 μl of HBSS buffer per well (monolayer integrity plate) A Lucifer Yellow (Sigma #L0144) solution was made up at 1001.1M in HBSS buffer. The empty inserts were transferred into the monolayer integrity plate and filled with 500 μl of the Lucifer Yellow solution to determine the cell monolayers integrity and leftover of the solution was kept in a fridge. The plate was incubated at 37° C. in a 5% CO₂ environment for 2 hours.

In the meantime, 150 μl were sampled from the assay plate (receiver solutions) and dispensed into 150 μl of HPLC-grade Acetonitrile containing an internal standard and 0.05% formic acid. A calibration curve was made for each mix of compounds (as appropriate depending on compounds molecular weights) A 10 μM mix of compounds in HBSS buffer was diluted 1:1 in HPLC-grade Acetonitrile containing an internal standard and 0.05% formic acid. A 1:1 mix of HBSS buffer and HPLC-grade Acetonitrile containing an internal standard and 0.05% formic acid was made up and used to make up the calibration curve (2 fold dilutions), with concentrations ranging from 10 μM to 0.078 μM.

After 2 hours, four wells of a black microtiter plate were filled with 50 μl HBSS and 50 μl of the 100 μM Lucifer Yellow solution to account for the initial solution fluorescence. Four wells were filled with 100 μl HBSS for the blank. Then 50 μl were sampled from the wells of the monolayer integrity plate, in duplicate, and dispensed into 50 μl HBSS in the black microtiter plate. The plate was read on a fluorescence reader (SpectraMax Gemini, Molecular Devices) setting the excitation wavelength at 430 nm and emission wavelength at 530 nm The monolayers integrity was assessed by the rejection of the Lucifer Yellow dye by tight monolayers. The following formula was applied to calculate rejection by each monolayer:

$$\% rejection = 100 \times 1 - ( \frac{RFU receiver}{RFU starting solution} )$$

using the mean fluorescence of the starting 100 μM Lucifer Yellow solution, and the fluorescence in the receiver solutions. All data are blank subtracted. The % rejection was considered very good if between 98 and 100% and good if between 96 and 98%. A rejection below 96% suggested that the monolayers were likely compromised during the assay.

The sampled receiver and donor solutions and calibration curves were analysed by HPLC-MS/MS (LCQuantum, Thermo Scientific) using a 50×2.1 mm i d Luna C18 5 μm column, 0.8 ml/min flow rate, and 5 μl injection volume. The HPLC gradient was 95% A (HPLC-grade water containing 0.05% (v/v) formic acid) 5% B (Acetonitrile containing 0.05% (v/v) formic acid) to 5% A, 95% B with a run of about 3 minutes.

Samples were processed using the Xcalibur software. Concentrations in the receiver solutions were used to calculate the apparent permeability coefficient (Papp) using the following formula: [Papp (cm²/sec)=receiver volume (ml)/[Area (cm²)×Time (sec)]×Cf/Ci], where receiver vol. is the volume in the receiver wells, Area is the surface of the inserts membrane, Time is the length of the permeability assay in seconds, Cf is the calculated final concentration of compound in the receiver solution and Ci is the known is initial concentration of compound in nM. The acceptance criteria for the control compounds were Papp of <1 cm²/sec for Atenolol, 5 to 10 cm²/sec for Dexamethasone, 18 to >20 cm²/sec for Propranolol.

The mass balance was calculated using the following formula: [Mass balance (%)=(final compound concentration in receiver solution+final compound concentration in donor solution)/(initial concentration of the donor solution)]. A Mass Balance greater than 70% was considered good. Results were accepted but flagged as biased when Mass Balance was less than 70%. Specimen results are shown in the following table.

### Permeability Assay Results:

| Example No. | Papp (×10⁻⁶ cm/sec) |
|---|---|
| 2 | 0.2 |
| 3 | 4.1 |
| 5 | 2 |
| 6 | 0.1 |
| 7 | 0.4 |
| 8 | 1.2 |
| 9 | 0.9 |
| 13 | 0.3 |
| 14 | 1.2 |
| 15 | 0.3 |
| 16 | 1 |
| 17 | 0.4 |
| 20 | 0.4 |
| 21 | 1.4 |
| 22 | 0.55 |
| 40 | 0.7 |
| 47 | 0.1 |
| 54 | 6 |

1. A compound of formula (I), or a pharmaceutically acceptable salt thereof, 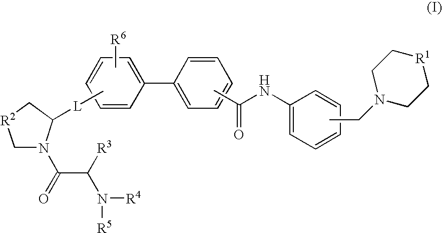
wherein
L represents a five membered heteroaromatic ring containing 1 to 3 heteroatoms independently selected from O, S and N;
R¹ represents SO₂, NSO₂R⁷ or NSO₂NR⁷R⁸;
R² represents a bond, CH₂, CH₂CH₂ or CH₂O;
R³ represents H, C1-4 alkyl, CH₂OH, CHOHCH₃ or Ph;
R⁴ represents H, C1-4 alkyl or CO₂R⁹;
R⁵ represents H or C1-4 alkyl;
R⁶ represents H, C1-2 alkyl, halogen or OCF₃;
R⁷ represents C1-4 alkyl; and
R⁸ and R⁹ independently represent H or C1-4 alkyl.

2. A compound, or a pharmaceutically acceptable salt thereof, according to claim 1 wherein R³ represents C1-4 alkyl, CH₂OH or Ph.

3. A compound, or a pharmaceutically acceptable salt thereof, according to claim 1 wherein the group L represents an imidazole ring.

4. A compound, or a pharmaceutically acceptable salt thereof, according to claim 1 wherein R¹ represents SO₂ or NSO₂R⁷ and R⁷ represents 1-propyl.

5. A compound, or a pharmaceutically acceptable salt thereof, according to claim 1 wherein R² represents CH₂, CH₂CH₂ or CH₂O.

6. A compound, or a pharmaceutically acceptable salt thereof, according to claim 1 wherein R³ represents H, methyl, ethyl, 1-propyl, 2-propyl, n-butyl, iso-butyl sec-butyl, tert-butyl, CH₂OH, CHOHCH₃ or Ph.

7. A compound, or a pharmaceutically acceptable salt thereof, according to claim 1 wherein R⁴ represents methyl, ethyl, CO₂-methyl or CO₂-tert-butyl and R⁵ represents H, methyl or ethyl.

8. A compound, or a pharmaceutically acceptable salt thereof, according to claim 1 wherein the compound of formula (I) is a compound of formula (Ia):

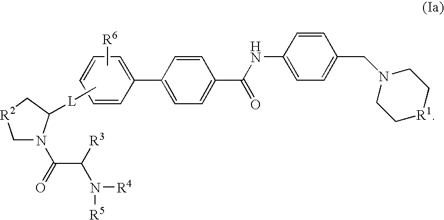

9. A compound, or a pharmaceutically acceptable salt thereof, according to claim 1 wherein the compound of formula (I) is a compound of formula (Ib):

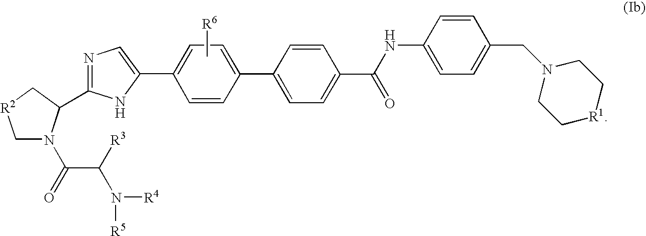

10. A compound, or a pharmaceutically acceptable salt thereof, according to claim 1 wherein the compound of formula (I) is a compound of formula (Id): 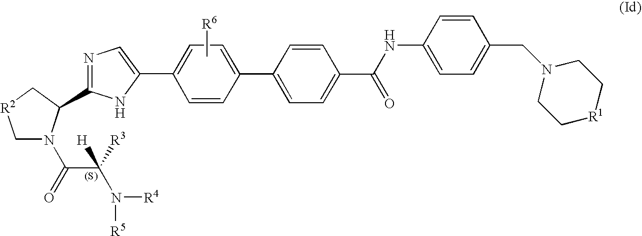
with the proviso that R³ is other than H.

11. A compound according to claim 1 selected from:
tert-butyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
tert-butyl N-[(1R)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxo-ethyl]carbamate;
Methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2,2-dimethyl-propyl]carbamate;
Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
Methyl N-[(1R)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
4-[4-[2-[(2S)-1-[(2R)-2-(diethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
4-[4-[2-[(2S)-1-[(2R)-2-(dimethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
4-[4-[2-[(2S)-1-[(2S)-2-amino-3-methyl-butanoyl]pyrrolidin-2-yl]-1H-imidazol-5-yl]phenyl]-N-[4-[(1,1-dioxo-1,4-thiazinan-4-yl)methyl]phenyl]benzamide;
Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-(hydroxymethyl)-2-oxo-ethyl]carbamate;
tert-Butyl N-[2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-ethyl]carbamate;
Methyl N-[(1R)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-3-methyl-butyl]carbamate;
Methyl N-[(1R)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-3-methyl-butyl]carbamate;
Methyl N-[(1S,2S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-butyl]carbamate;
Methyl N-[(1S,2R)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-hydroxy-propyl]carbamate;
Methyl N-[(1R,2S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-hydroxy-propyl]carbamate;
Methyl N-[(1R)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-(hydroxymethyl)-2-oxo-ethyl]carbamate;
Methyl N-[(1S)-1-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-2-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxo-ethyl]carbamate;
Methyl N-[(1S)-1-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2,2-dimethyl-propyl]carbamate;
Methyl N-[(1S)-2-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
Methyl N-[(1R)-2-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
4-[4-[2-[(2R)-1-[(2R)-2-(diethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
4-[4-[2-[(2R)-1-[(2R)-2-(dimethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
methyl N-[(1R)-1-[(2R)-2-[5-[4-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-1-[(2S)-2-[5-[4-[3-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-2-[(2S)-2-[5-[4-[3-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxo-ethyl]carbamate;
Methyl N-[(1S)-2-[(2S)-2-[5-[4-[3-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
Methyl N-[(1R)-2-[(2S)-2-[5-[4-[3-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
3-[4-[2-[(2S)-1-[(2R)-2-(diethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
Methyl N-[(1S)-1-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2-methyl-propyl]carbamate;
Methyl N-[(1S)-2-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidin-1-yl]-1-methyl-2-oxo-ethyl]carbamate;
Methyl N-[(1S)-1-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidine-1-carbonyl]-2,2-dimethyl-propyl]carbamate;
Methyl N-[(1S)-2-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
Methyl N-[(1R)-2-[(2S)-2-[5-[3-[4-[[4-[(1,1-dioxo-1,4-thiazinan-4-yl)methyl]phenyl]carbamoyl]phenyl]phenyl]-1H-imidazol-2-yl]pyrrolidin-1-yl]-2-oxo-1-phenyl-ethyl]carbamate;
4-[3-[2-[(2S)-1-[(2R)-2-(diethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
4-[3-[2-[(2S)-1-[(2R)-2-(dimethylamino)-2-phenyl-acetyl]pyrrolidin-2-yl]-1H-imidazol-5-yl]phenyl]-N-[4-[(1,1-dioxo-1,4-thiazinan-4-yl)methyl]phenyl]benzamide;
Methyl N-[(1S)-2-methyl-1-[(2S)-2-[5-[4-[4-[[4-[(4-propylsulfonylpiperazin-1-yl)methyl]phenyl]carbamoyl]phenyl]phenyl]-1H-imidazol-2-yl]pyrrolidine-1-carbonyl]propyl]carbamate;
Methyl N-[(1S)-1-methyl-2-oxo-2-[(2S)-2-[5-[4-[4-[[4-[(4-propylsulfonylpiperazin-1-yl)methyl]phenyl]carbamoyl]phenyl]phenyl]-1H-imidazol-2-yl]pyrrolidin-1-yl]ethyl]carbamate;
Methyl N-[(1S)-2-oxo-1-phenyl-2-[(2S)-2-[5-[4-[4-[[4-[(4-propylsulfonylpiperazin-1-yl)methyl]phenyl]carbamoyl]phenyl]phenyl]-1H-imidazol-2-yl]pyrrolidin-1-yl]ethyl]carbamate;
Methyl N-[(1R)-2-oxo-1-phenyl-2-[(2S)-2-[5-[4-[4-[[4-[(4-propylsulfonylpiperazin-1-yl)methyl]phenyl]carbamoyl]phenyl]phenyl]-1H-imidazol-2-yl]pyrrolidin-1-yl]ethyl]carbamate;
4-[4-[2-[(2S)-1-[(2R)-2-(diethylamino)-2-phenyl-acetyl]pyrrolidin-2-yl]-1H-imidazol-5-yl]phenyl]-N-[4-[(4-propylsulfonylpiperazin-1-yl)methyl]phenyl]benzamide;
4-[4-[2-[(2S)-1-[(2R)-2-(dimethylamino)-2-phenyl-acetyl]pyrrolidin-2-yl]-1H-imidazol-5-yl]phenyl]-N-[4-[(4-propylsulfonylpiperazin-1-yl)methyl]phenyl]benzamide;
Methyl N-[(1S)-1-[(3R)-3-[5-[4-[4-[[4-[(1,1-dioxo-1,4-thiazinan-4-yl)methyl]phenyl]carbamoyl]phenyl]phenyl]-1H-imidazol-2-yl]morpholine-4-carbonyl]-2-methyl-propyl]carbamate;
Methyl N-[(1S)-2-[(3R)-3-[5-[4-[4-[[4-[(1,1-dioxo-1,4-thiazinan-4-yl)methyl]phenyl]carbamoyl]phenyl]phenyl]-1H-imidazol-2-yl]morpholin-4-yl]-2-oxo-1-phenyl-ethyl]carbamate;
tert-Butyl N-[(1S)-2-[(3R)-3-[5-[4-[4-[[4-[(1,1-dioxo-1,4-thiazinan-4-yl)methyl]phenyl]carbamoyl]phenyl]phenyl]-1H-imidazol-2-yl]morpholin-4-yl]-2-oxo-1-phenyl-ethyl]carbamate;
4-[4-[2-[(3R)-4-[(2R)-2-(dimethylamino)-2-phenyl-acetyl]morpholin-3-yl]-1H-imidazol-5-yl]phenyl]-N-[4-[(1,1-dioxo-1,4-thiazinan-4-yl)methyl]phenyl]benzamide;
methyl N-[(1S)-1-[(2S)-2-[5-[4-[4-[[4-[(1,1-dioxo-1,4-thiazinan-4-yl)methyl]phenyl]carbamoyl]phenyl]phenyl]-1H-imidazol-2-yl]piperidine-1-carbonyl]-2-methyl-propyl]carbamate;
Methyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]-1-piperidyl]-2-oxo-1-phenyl-ethyl]carbamate;
tert-Butyl N-[(1S)-2-[(2S)-2-[5-[4-[4-[[4-[(1,1-dioxo-1,4-thiazinan-4-yl)methyl]phenyl]carbamoyl]phenyl]phenyl]-1H-imidazol-2-yl]-1-piperidyl]-2-oxo-1-phenyl-ethyl]carbamate;
4-[4-[2-[(2S)-1-[(2R)-2-(dimethylamino)-2-phenyl-acetyl]-2-piperidyl]-1H-imidazol-5-yl]phenyl]-N-[4-[(1,1-dioxo-1,4-thiazinan-4-yl)methyl]phenyl]benzamide;
Methyl N-[(1S)-1-[(2S)-2-[5-[4-[4-[[4-[(1,1-dioxo-1,4-thiazinan-4-yl)methyl]phenyl]carbamoyl]phenyl]-3-methyl-phenyl]-1H-imidazol-2-yl]pyrrolidine-1-carbonyl]-2-methyl-propyl]carbamate;
or a pharmaceutically acceptable salt thereof.

12. A method of treating, or reducing the risk of HCV which comprises administering to a patient in need thereof a therapeutically effective amount of a compound of formula (I), as defined in claim 1, or a pharmaceutically acceptable salt thereof.

13. A pharmaceutical composition comprising a compound of formula (I), as defined in claim 1, or a pharmaceutically acceptable salt thereof, and a pharmaceutical acceptable diluent or carrier.

14. A combination comprising:
a compound of formula (I) as defined in claim 1 or a pharmaceutically acceptable salt thereof;
a HCV protease inhibitor and/or a HCV polymerase inhibitor;
an interferon; and
ribavarin.

15. A process for the preparation of a compound of formula (I) as defined in claim 1, or a pharmaceutically acceptable salt thereof, which comprises a process (a), (b) or (c) wherein, unless otherwise defined, the variables are as defined in claim 1 for compounds of formula (I):
(a) reacting a compound of formula (II): 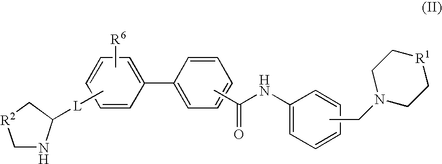
with a compound of formula (III): 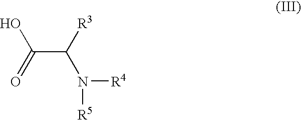
(b) reacting a compound of formula (IV): 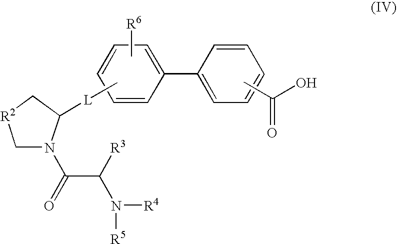
or
with a compound of formula (V): 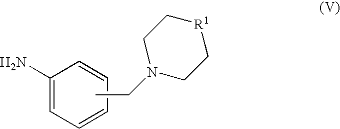
Or
(c) reacting together compounds of formulae (VI) and (VII): 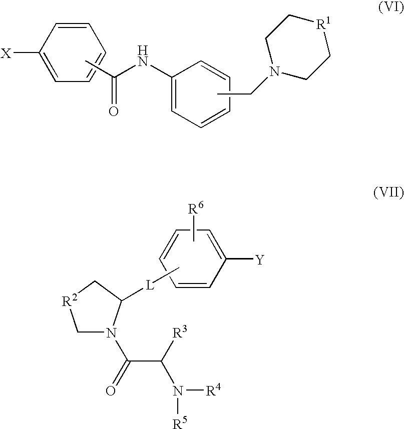
wherein either X represents halogen and Y represents —B(OH)₂ or an ester thereof; or Y represents halogen and X represents —B(OH)₂ or an ester thereof;
and optionally after (a), (b) or (c) carrying out one or more of the following:
converting the compound obtained to a further compound of the invention
forming a pharmaceutically acceptable salt of the compound.
